(19) United States
(12) Patent Application Publication
(10) Pub. No.: US 2010/0197006 A1
(43) Pub. Date: Aug. 5, 2010

(54) MOLECULAR CIRCUITS

(75) Inventors: Yaakov Benenson, Watertown, MA (US); Ron Weiss, Newton, MA (US); Leonidas Bleris, Allen, TX (US); Zhen Xie, Malden, MA (US)

Correspondence Address: DAVID S. RESNICK, NIXON PEABODY LLP, 100 SUMMER STREET, BOSTON, MA, 02110-2131, US

(73) Assignees: PRESIDENT AND FELLOWS OF HARVARD COLLEGE, Cambridge, MA (US); TRUSTEES OF PRINCETON UNIVERSITY, Office of Technology Licensing and Intellectual Property, Princeton, NJ (US)

(21) Appl. No.: 12/605,040

(22) Filed: Oct. 23, 2009

Related U.S. Application Data
(63) Continuation of application No. PCT/US2008/061687, filed on Apr. 25, 2008.
(60) Provisional application No. 60/913,937, filed on Apr. 25, 2007.
(60) Provisional application No. 60/942,807, filed on Jun. 8, 2007.
(60) Provisional application No. 61/008,118, filed on Dec. 17, 2007.
(60) Provisional application No. 61/067,757, filed on Feb. 28, 2008.

(51) Int. Cl. C12N 5/10; C12N 1/19

(52) U.S. Cl. 435/325; 435/419; 435/254.2

(57) ABSTRACT

Disclosed herein are autonomous molecular circuits that can function in cells. The circuits can process logical operations in which one or more input cues are among the operands and produce an appropriate output. Such circuits can be implemented in living cells, e.g., eukaryotic or prokaryotic cells that have been modified to include circuit components. The molecular circuits and cells containing the circuits can be used in a variety of applications including, e.g., diagnostics, therapeutics, and protein production.

### CROSS-REFERENCE TO RELATED APPLICATIONS

This application is a Continuation Application which claims benefit under 35 U.S.C. §120 of U.S. International Application No. PCT/US2008/061687 filed Apr. 25, 2008, which designated the U.S., the contents of which are herein incorporated in their entirety by reference, and which claims benefit under 35 U.S.C. §119(e) to U.S. Provisional Application Nos. 60/913,937 filed Apr. 25, 2007, 60/942,807 filed Jun. 8, 2007, 61/008,118 filed Dec. 17, 2007, and 61/067,757 filed Feb. 28, 2008, the contents of all of which are herein incorporated in their entirety by reference.

The research described in this application was supported by grant no. 5P50 GM068763-01 from the National Institute of General Medical Sciences (NIGMS) of the National Institutes of Health. The United States government may have certain rights in the invention.

### SEQUENCE LISTING

The instant application contains a Sequence Listing which has been submitted via EFS-Web and is hereby incorporated by reference in its entirety. Said ASCII copy, created on Feb. 18, 2010, is named 00280606.txt, and is 15,926 bytes in size.

### SUMMARY

This disclosure features, inter alfa, cells engineered to process multiple parameters, for example, using Boolean logic in order to produce a response. Circuit components can be coupled to internal or external inputs and can be combined in order to process any arbitrary expression.

In one aspect, the disclosure provides a molecular circuit includes: (1) a plurality of mediators, the activity of each mediator being a function of an input cue; and (2) an output that is regulated by the mediators. The output is the result of a Boolean operation for which the input cues are operands. The molecular circuit can be coupled to at least two input cues and can generate an output. The molecular circuit can include two or more input cues, e.g., at least three or four input cues. The molecular circuit can include more than one output or more than one mediator. The disclosure also features a modified cell (e.g., a modified eukaryotic cell) that includes a molecular circuit. The modified cell can be, e.g., a fungal cell, a plant cell, or animal cell. The modified cell can be a mammalian cell (e.g., a non-human mammalian cell or a human cell) or any other type of eukaryotic cell described herein. The input cues can be evaluated in parallel.

In some embodiments, at least one mediator can regulate mRNA or protein expression of an output. For example, at least one mediator can be a functional nucleic acid, e.g., an siRNA, an miRNA, or an shRNA. The activity (or stability) of the siRNA, e.g., can depend on (i) the presence or absence of a small molecule that specifically binds to at least one strand of the siRNA; (ii) the presence or absence of a protein that specifically binds to at least one strand of the siRNA; or (iii) the presence or absence of an mRNA that specifically binds to at least one strand of the siRNA.

In some embodiments, the mediator can be genetically coded. For example, the mediator can be a biopolymer such as, but not limited to, an siRNA, an miRNA, an shRNA, an mRNA, or a protein.

In some embodiments, the molecular circuit can contain two or more (e.g., two, three, four, five, six, seven, eight, nine, 10, 11, 12, 15, 20, 25, or 30 or more) mediators. The molecular circuit can contain, e.g., two or more of the same type of mediator. For example, a circuit can contain two or more siRNA, miRNA, shRNA, mRNA, or protein mediators. A molecular circuit can contain two or more (e.g., two, three, four, five, six, seven, eight, nine, 10, 11, 12, 15, 20, 25, or 30 or more) siRNA mediators. A molecular circuit can contain two or more (e.g., two, three, four, five, six, seven, eight, nine, 10, 11, 12, 15, 20, 25, or 30 or more) shRNA mediators. A molecular circuit can contain two or more (e.g., two, three, four, five, six, seven, eight, nine, 10, 11, 12, 15, 20, 25, or 30 or more) miRNA mediators. A molecular circuit can contain two or more (e.g., two, three, four, five, six, seven, eight, nine, 10, 11, 12, 15, 20, 25, or 30 or more) mRNA mediators. A molecular circuit can contain two or more (e.g., two, three, four, five, six, seven, eight, nine, 10, 11, 12, 15, 20, 25, or 30 or more) protein mediators. The molecular circuit can contain two or more different mediators. For example, the molecular circuit can contain, e.g., one siRNA mediator and one protein mediator or two or more protein mediators and two or more miRNA mediators.

In some embodiments, at least one input cue can be a protein, an mRNA, a small molecule, an output of another molecular circuit in the cell, or the output of another molecular circuit in another cell.

In some embodiments, the output can be an mRNA or a protein. The protein can be a reporter protein such as ZsYellow or DsRed. An output protein can be encoded by an mRNA comprising at least one response element for a mediator. The mRNA can comprise a 3′ or 5′-untranslated region (UTR), the region comprising a response element for at least one mediator. In some embodiments, at least one mediator can be an siRNA, and stability of the mRNA depends on whether the mediator that is an siRNA causes cleavage of the 3′ UTR. In some cases, the output can alter a cellular activity of the modified cell. For example, the alteration in cellular activity can cause or alter apoptotic cell death, replication (e.g., DNA or cellular replication), cell differentiation, or cell migration. In some embodiments, the output can be a pharmaceutical agent that has a therapeutic property. In some embodiments, the output can be a functional nucleic acid, e.g., an siRNA, an miRNA, or shRNA. In some embodiments, the output can be a light modulating/producing protein. Where multiple distinct outputs are created, one or more such outputs can be a light modulating/producing protein. For example, two or more such outputs can be light modulating/producing proteins, each being distinguishable from the other, e.g., by having distinguishable spectra.

An output can be directly or indirectly mediated by at least one mediator. For example, the output can be regulated indirectly by a regulatory protein, which regulatory protein is regulated by at least one mediator. The regulatory protein can be a transcription factor, a kinase, a phosphatase, a glycosylase, or a GTPase. The transcription factor can regulate the expression of an output mRNA or an output protein.

Mediators can be provided in a variety of fashions. For example, at least one mediator, or all mediators, can be encoded by one or more heterologous genes introduced into the cell. The heterologous genes can be introduced transiently or in a stable fashion. The heterologous gene can be dependent on a promoter of a cellular gene. The mediators can themselves be introduced into cells. Mediators can be coupled to input cues in a variety of manners. For example, at least one mediator can be positively or negatively regulate by at least one input cue.

The molecular circuit can include one or more AND circuits, e.g., at least two, three or four AND circuits. The molecular circuit can include one or more OR circuits, e.g., at least two, three or four OR circuits. Combinations of such circuits are also possible, e.g., at least one AND circuit and at least two OR circuits; or at least two AND circuits and at least three OR circuits.

In one embodiment, the molecular circuit implements an expression in a conjunctive normal form, e.g., (A₁₁ OR A₂₁ OR . . . A_{N}⁽¹⁾₁) AND (A₁₂ OR A₂₂ OR . . . A_{N}⁽²⁾₂) AND . . . (A_{K1} OR A_{K2} OR . . . A_{N}^{(K)}_{K}), where A_{IJ} is either a logic representation of the presence of a molecular input cue or a logic representation of its absence, NOT(cue); K is the number of “AND” switches; and N^{(I)} is a number of literals that are combined in an I'th OR switch. In another embodiment, the molecular circuit implements expressions in a disjunctive normal form, e.g., (A₁₁ AND A₂₁ AND . . . A_{N}⁽¹⁾₁) OR (A₁₂ AND A₂₂ AND . . . A_{N}⁽²⁾₂) OR . . . (A_{K1} AND A_{K2} AND . . . A_{N}^{(K)}_{K}), where A_{IJ} is either a logic representation of the presence of a molecular input cue or a logic representation of its absence, NOT(cue); K is the number of “OR” switches; and N^{(I)} is a number of literals that are combined in an I'th AND switch.

In some embodiments, at least one input cue can be an endogenous input cue or an exogenous input cue. The endogenous input cue can be the presence of an oncogene or the absence of a tumor suppressor protein. The exogenous input cue can be from an intracellular bacterium, a virus, or an intracellular parasite.

Exemplary circuits have minimal cross-talk and noise. In many implementations, the circuits have at least a two, three, four, ten, 15- or 16-fold average difference between TRUE and FALSE output levels.

In some embodiments of any of the circuits described herein, the molecular circuits are not regulated by, or coupled to, a nuclease (e.g., an endonuclease or an exonuclease) such as, e.g., a DNA endonuclease (e.g., a restriction enzyme).

In another aspect, the disclosure provides a modified eukaryotic cell that includes an exogenous molecular circuit. The molecular circuit is coupled to at least two input cues and generates an output. The molecular circuit includes: (1) a plurality of siRNA mediators, the activity of each siRNA mediator being responsive to an input cue; and (2) an mRNA that encodes an output. The mRNA includes a response elements for each of the siRNA mediators. The circuit can contain more than one mRNA. The circuit can include other features described herein.

In some embodiments, the response element can be in the coding region of the mRNA. In some embodiments, the response element can be in a non-coding region of the mRNA such as the 3′UTR region or the 5′ UTR region of an mRNA. In some embodiments, the 3′UTR region or the 5′ UTR region of an mRNA can contain a response element for at least one siRNA mediator. Components can be provided such that the presence, or the absence, of one of the input cues can cause cleavage of the mRNA.

In some embodiments, the modified cell can further include a second Boolean circuit operator for which the output of the first molecular circuit is an input cue. Multiple circuit components for Boolean switches can be combined in this fashion to provide a complex circuit that processes a Boolean operation with greater than two literals, e.g., greater than three, four, or five literals.

In yet another aspect, the disclosure features a modified eukaryotic cell, the cell including an exogenous molecular circuit. The molecular circuit is coupled to at least two, three, or four input cues and generates an output. The molecular circuit includes: (1) at least two siRNA mediators, the activity of each mediator being the function of an input cue; (2) one or more molecular switches coupled to the siRNA mediators, the switches being configured to perform Boolean operations for the input cues are operands; and (3) an output protein, the translation of which is regulated by the switches such that production of the output is the result of any pre-defined Boolean expression operation.

In some embodiments, at least one of the molecular switches (2) is a Boolean AND switch, wherein (a) the switch includes an mRNA encoding the output protein, (b) each of the siRNA mediators is active only in the absence of its respective input cue; and (c) the mRNA includes response elements for the siRNA mediators, such that cleavage of the mRNA by any one of the siRNA mediators prevents translation of the output. The response elements can be located in the 3′ or 5′ untranslated region of the mRNA.

In some embodiments, (a) the circuit includes a Boolean OR switch that consists of a plurality of mRNAs (2) encoding the output protein, and (b) each of the mRNAs being coupled to at least one different siRNA mediator.

In some embodiments, the circuit includes a Boolean OR switch that operands for which a first Boolean AND switch and a second Boolean AND switch, the Boolean OR switch including two mRNAs encoding the output protein, each of the Boolean AND switches including response elements for siRNA mediators located in of the mRNAs encoding the output protein.

In some embodiments, at least one of the molecular switches regulates translation of a first regulatory protein, and at least another regulates translation of a second regulatory protein, and an active regulator that regulates gene expression of an mRNA encoding the output protein is formed only when the first and second regulatory proteins interact. The first and second regulatory proteins can form a heterodimer, such as a homodimeric transcription factor. The transcription factor can represses or activate gene expression of the mRNA encoding the output protein. In some embodiments, the first regulatory protein can include a eukaryotic transcription activation domain and a protein interaction region, and second regulatory protein includes a DNA binding domain and a region that binds to the protein interaction region of the first regulatory protein.

In some embodiments, the molecular circuit is coupled an input cue and generates an output, the molecular circuit comprising: (1) a plurality of mediators, the activity of each mediator being a function of an input cue; (2) two or more (e.g., three, four, five, six, seven, eight, nine, or 10 or more) molecular switches coupled to the mediators, the switches being configured to perform Boolean operations wherein the input cues are operands; and (3) an output that is regulated by the switches.

In another aspect, the disclosure provides a method of designing a molecular circuit. The method can include providing a modified cell including mediator components responsive to input cues in a configuration that regulates an output component according to one or more logical switches to which the mediator components can be coupled. For example, the circuit can include one or more other features described herein.

In some embodiments, the providing can include, e.g., (i) introducing one or more heterologous genes that encode the mediator component or output component into the cell; (ii) introducing one or more heterologous genes that encode the mediator component into the cell; (iii) introducing one or more heterologous genes that encode the output component into the cell; and/or (iv) introducing one or more heterologous genes that encode the switch component into the cell.

In some embodiments, at least one of the mediator components can respond to (i) the presence of a protein as its input cue; (ii) the expression of an mRNA as its input cue; (iii) the presence of a small molecule as its input cue; and/or (iv) an extracellular signal as its input cue. The output can be a drug molecule or a compound that can trigger release of a drug molecule. The output can be a reporter agent, e.g., a reporter protein or mRNA.

In another aspect, the disclosure provides a method of designing a molecular circuit. The method includes the steps of: selecting mediator components that respond to input cues; selecting a logical switch to which the mediator components can be coupled; and providing a nucleic acid vector including one or more nucleic acids sufficient to produce the logical switch components in a eukaryotic cell

In yet another aspect, the disclosure also features a vector including one or more nucleic acids sufficient to effectuate in a eukaryotic cell the components of any molecular circuit described herein. The vector can be a viral vector such as a retroviral vector (e.g., a lentiviral vector), a adenoviral vector, or a baculoviral vector.

In another aspect, the disclosure provides a kit containing the above-mentioned vector and, optionally, instructions for introducing the vector into a eukaryotic cell and/or instructions for detecting an output of the circuit.

In an additional aspect, the disclosure features a method of designing a molecular circuit, which method includes the steps of: expressing a diagnostic rule as Boolean operations in disjunctive or conjunctive normal form; and modifying a cell to include molecular logic circuits corresponding to each Boolean operation in the rule as expressed in the normal form. The method can optionally include the steps of: expressing the diagnostic rule in both disjunctive and conjunctive normal form; and selecting the form that includes fewer literals. The diagnostic rule can be coupled to a reporter or a therapeutic.

In yet another aspect, the disclosure provides a molecular circuit designed using any of the method described herein and, optionally, instructions for how to introduce the circuit into a cell and/or instructions for detecting an output of the circuit.

In another aspect, the disclosure features an mRNA-sensor molecular circuit responsive to an mRNA cue, which circuit includes: (i) a first siRNA component and second siRNA component; (ii) an aptamer that binds to the first siRNA component and prevents the formation of the active siRNA; and (iii) an mRNA cue. The association of the first and second siRNA components forms an active siRNA. The mRNA, if present, specifically binds to the aptamer thereby allowing the formation of the active siRNA. The siRNA can regulate an output. The first and second siRNA components can be covalently joined such as in an shRNA. siRNA aptamers can be encoded by recombinant nucleic acids, e.g., DNA that can be integrated into the cellular genome.

In some embodiments, the mRNA can be an endogenous mRNA or an exogenous mRNA. The endogenous mRNA can encode an oncogene or a mutant form of a tumor suppressor protein. The exogenous mRNA can be from an intracellular bacterium, a virus, or an intracellular parasite.

In another aspect, the disclosure provides a recombinant cell including exogenous nucleic acids that express the nucleic acid components of an mRNA circuit described above.

In yet another aspect, the disclosure features a protein-sensor molecular circuit responsive to a protein cue, the circuit including: (i) a first siRNA component and second siRNA component; (ii) an aptamer that binds to the first siRNA component; and (iii) a protein cue. The association of the first and second siRNA components forms an active siRNA. The binding of the aptamer to the first siRNA component to the aptamer prevents the formation of the active siRNA The protein, if present, specifically binds to the aptamer thereby allowing the first siRNA to form the active siRNA with the second siRNA. The siRNA can regulate an output. The first and second siRNA components can be covalently joined such as in an shRNA. The siRNA and/or the aptamer can be encoded by recombinant nucleic acids.

In another aspect, the disclosure provides a protein-sensor molecular circuit responsive to a protein cue. The circuit contains: (i) a first siRNA component and second siRNA component; and (ii) a protein cue. The association of the first and second siRNA components forms an active siRNA The protein, if present, binds to the first or second siRNA component thereby preventing the formation of the active siRNA. The siRNA can regulate an output. The first and second siRNA components can be covalently joined such as in an shRNA.

In another aspect, the disclosure provides a kit including any of the mRNA or protein-sensor molecular circuits described herein, or constructs and other reagents for preparing components of such circuits, and, optionally, instructions, e.g., instructions for monitoring the circuit into a cell.

In one example, a multi-component RNA- and/or protein-based biomolecular system is engineered to detect complex conditions related to abnormal expression of a number of arbitrary genes in mammalian cells, and to release an arbitrary biologically active protein upon detection. The system operates as a molecular automaton and can include one or more of: (1) molecular sensors that assess the levels of gene expression; (2) a molecular computation module that integrates the information related by the sensors and makes a diagnostic decision; and (3) a molecular actuator module that translates the output of the molecular computation into biological action.

In another aspect, the disclosure features a molecular circuit (such as any one of the molecular circuits described herein) that includes a molecular band-pass filter. For example, the filter can include one or more (e.g., two, three, four, five, six, seven, eight, nine, 10, 11, 12, 13, 15, 20, or 25 or more) filter effectors that regulate the amount of an output generated by the circuit in response to one or more input cues. Examples of filter effectors include an siRNA, an miRNA, an shRNA, or a protein (such as a transcription repressor). Combinations of any of the foregoing can be used.

The molecular band-pass filter can regulate the amount of an output within a predetermined range. In some embodiments, the molecular band-pass filter can prevent or decrease the likelihood that the amount of an output exceeds a predetermined threshold level. In some embodiments, the molecular band-pass filter can prevent or decrease the likelihood that the amount of an output falls below a predetermined threshold level.

In some embodiments, the molecular band-pass filter can be activated when the amount of an output falls below a predetermined level and/or when the amount of the output exceeds a predetermined range.

In some embodiments, the molecular band-pass filter can contain at least one filter effector that negatively regulates the output of the circuit. In some embodiments, the circuit can contain at least one filter effector that positively regulates the output of the circuit. In some embodiments, the circuit can contain at least one filter effector that antagonizes the action of a mediator of the circuit. For example, a circuit can contain (i) a transcription activator that positively regulates the expression of an output protein and (ii) a shRNA filter effector, which binds to and promotes the degradation of an mRNA that encodes the output protein.

In some embodiments, a filter effector can be encoded on the same DNA cassette as a mediator. In some embodiments, a filter effector can be encoded on different cassette than a mediator. In some embodiments (e.g., in embodiments where the filter effector antagonizes a mediator), a filter effector and mediator can be responsive to the same input cue(s).

A molecular circuit, and the band-pass filter, can include other features described herein. In a typical example, the molecular circuit is in a cell, e.g., a mammalian cell.

In another aspect, the disclosure features a cell that includes recombinant genetic information encoding at least two (e.g., three, four, five, six, seven, eight, nine, or 10 or more) filter effectors that are correlated and that target a common molecule or pathway. The two filter effectors can antagonize each other, e.g., one activates expression of a gene, whereas the other destabilizes the mRNA transcribed by the gene. In another example, a first filter effector can be an mRNA and the second filter effector can be an miRNA. In this case, the first and second filter effector can be transcribed as a fusion transcript from separate genes or can be transcribed as one transcript from the same gene. In some embodiments, the miRNA can be liberated from the mRNA portion of the fusion transcript by splicing.

In some embodiments, the common molecule can be, e.g., the output of a molecular circuit such as any of the molecular circuits described herein. The common molecule can be, e.g., a regulatory protein that regulates the production of an output.

The at least two filter effectors can regulate the amount of an output of a molecular circuit within a predetermined range. In some embodiments, the at least two filter effectors can prevent or decrease the likelihood that the amount of an output exceeds a predetermined threshold level. In some embodiments, the at least two filter effectors can prevent or decrease the likelihood that the amount of an output falls below a predetermined threshold level.

In some embodiments, the at least two filter effectors can be activated when the amount of an output falls below a predetermined level and/or when the amount of the output exceeds a predetermined range.

Additional features and capabilities of the at least two filter effectors are described herein.

In yet another aspect, the disclosure features a method for designing a molecular band-pass filter for any of the molecular circuits described herein, the method comprising: optionally providing a molecular circuit described herein, and designing for the circuit a molecular band-pass filter comprising one or more (e.g., two, three, four, five, six, seven, eight, nine, 10, 11, 12, 13, 15, 20, or 25 or more) filter effectors that regulate the amount of an output generated by the circuit in response to one or more input cues. The molecular band-pass filter can contain, or the one or more filter effectors can be, e.g., an siRNA, an miRNA, an shRNA, a protein such as a transcription repressor), or combinations of any of the foregoing.

The molecular band-pass filter can maintain the amount of an output within a predetermined range. In some embodiments, the molecular band-pass filter can be one that prevents or decreases the likelihood that the amount of an output exceeds a predetermined threshold level. In some embodiments, the molecular band-pass filter can be one that prevents or decreases the likelihood that the amount of an output falls below a predetermined threshold level.

In some embodiments, the molecular band-pass filter can be activated when the amount of an output falls below a predetermined level and/or when the amount of the output exceeds a predetermined range.

In some embodiments, the band-pass filter can contain at least one filter effector that negatively regulates the output of the circuit. In some embodiments, the band-pass filter can contain at least one filter effector that positively regulates the output of the circuit. In some embodiments, the circuit can contain at least one filter effector that antagonizes the action of a mediator of the circuit. For example, a circuit can contain (i) a transcription activator that positively regulates the expression of an output protein and (ii) a shRNA filter effector, which binds to and promotes the degradation of an mRNA that encodes the output protein.

In some embodiments, a filter effector can be encoded on the same DNA cassette as a mediator. In some embodiments, a filter effector can be encoded on different cassette than a mediator. In some embodiments (e.g., in embodiments where the filter effector antagonizes a mediator), a filter effector and mediator can be responsive to the same input cue(s).

In some embodiments, the method can further include determining whether the molecular band-pass filter regulates the amount of an output generated by the circuit in response to one or more input cues.

As described herein, any of the molecular circuits can be in, or be introduced into, a eukaryotic cell. The eukaryotic cell can be in culture (e.g., in a mixture of cells in culture) or can be in an organism. For example, the cell can be in a non-human animal (e.g., a non-human mammal). In some embodiments, the cell can be in a human.

In yet another aspect, the disclosure features a method of diagnosis. The method includes the steps of introducing into a cell any of the molecular circuits described herein and detecting an output. In some embodiments, the circuit is coupled an input cue and generates an output and the molecular circuit comprises a plurality of mediators, the activity of each mediator being a function of an input cue; two or more molecular switches coupled to the mediators, the switches being configured to perform Boolean operations wherein the input cues are operands; and an output that is regulated by the switches. The method can be useful for a variety of diagnoses including, e.g., cancer, metabolic diseases, infections, inflammatory disorder or any other diagnostic described herein.

In another aspect, the disclosure features a method of treatment. The method includes the step of delivering to at least one cell in a mammal in need thereof any of the molecular circuits described herein. The molecular circuit can be coupled an input cue and generate an output. The molecular circuit can contain a plurality of mediators, the activity of each mediator being a function of an input cue; two or more molecular switches coupled to the mediators, the switches being configured to perform Boolean operations wherein the input cues are operands; and an output that is regulated by the switches. The mammal can be one having a condition and the output is a therapeutic useful in treating, or ameliorating one or more symptoms of, the condition. The condition can be selected from the group consisting of a cancer, a metabolic disorder, an immunological disorder, and an infection.

In another aspect, the disclosure features a method of treatment, the method comprising administering to a mammal in need thereof a vector comprising a nucleic acid encoding one or more components of any of the molecular circuits described herein. The molecular circuit can be coupled an input cue and generate an output. The molecular circuit can contain a plurality of mediators, the activity of each mediator being a function of an input cue; two or more molecular switches coupled to the mediators, the switches being configured to perform Boolean operations wherein the input cues are operands; and an output that is regulated by the switches. The mammal can be one having a condition and the output is a therapeutic useful in treating, or ameliorating one or more symptoms of, the condition. The condition can be selected from the group consisting of a cancer, a metabolic disorder, an immunological disorder, and an infection.

In some embodiments, the vector can be, e.g., a virus, a plasmid, or any other vector described herein. In some embodiments, the vector can be in a cell and the cell delivered to the subject.

Decision-making automata can be used for the detection of and response to biologically important events in vivo in the contexts of basic research and medicine. Both normal (e.g., development) and anomalous (e.g., malignant transformations) biological events generate detectable changes in the molecular composition of the biological system, providing a number of “binary” endogenous molecular decision cues.

All publications, patent applications, patents, and other references mentioned herein are incorporated by reference in their entirety.

### BRIEF DESCRIPTION OF THE DRAWINGS

FIGS. 1A-1D depict (i) truth tables for the AND (conjunction), OR (disjunction), and NOT (negation) operations (FIG. 1A); (ii) Examples of two equivalent representations of an expression in disjunction (DNF) and conjunction (CNF) normal forms (FIGS. 2A and 2B). Any Boolean expression can be represented in these forms. “DNF” refers to the disjunction of several clauses, each containing only conjunctions of Boolean variables and their negations (literals). “CNF” refers to the conjunction of clauses containing only disjunctions of literals. Clauses and literals are indicated. An example evaluation of a Boolean expression in (FIG. 1B) for a given set of truth values is depicted in FIG. 1D.

FIGS. 2A and 2B depict an exemplary mechanism by which an siRNA mediator can respond to an endogenous mRNA cue.

FIGS. 3A and 3B depict an exemplary mechanism by which an siRNA mediator can respond to an endogenous protein cue.

FIGS. 4A-4C depict exemplary mechanisms of downregulation of siRNA derivatives by an mRNA molecule. (FIG. 4A) A synthetic shRNA sensor is expressed in situ from a suitable vector (r6). (FIG. 4B) A modification of the above mechanism that relies on the initial hybridization in the loop region rather than 5′-overhang. (FIG. 4C) An inactivation of a double-stranded siRNA structure.

FIGS. 5A-5E depicts exemplary molecular circuits having (i) a single output protein (ZsYellow protein); (ii) an mRNA encoding the protein, which mRNA contains one response element for a single siRNA mediator; (iii) an siRNA mediator, and (iv) an input cue that regulates (negatively (FIG. 5A, left) or positively (FIG. 5A, right)) the mediator. The exemplary molecular circuit contains: (i) an output protein (ZsRed protein); (ii) an mRNA encoding the output protein, (iii) a regulatory protein (LacI), (iv) an mRNA encoding the regulatory protein, which mRNA contains one response element for a single siRNA mediator; (v) an siRNA mediator, and (vi) an input cue that regulates (negatively (FIG. 5B, right) or positively (FIG. 5B, left)) the mediator. FIG. 5D depicts a molecular OR circuit containing: (i) two identical output proteins (ZsYellow proteins); (ii) two mRNAs, each encoding one of the two output proteins, and each mRNA containing multiple response elements (e.g., Target-a, Target-c, OR Target-e; and Target-NOT(a) AND Target-b, respectively) for different siRNA mediators; (iv) five siRNA mediators (siRNA-a, siRNA-NOT(a), siRNA-c, siRNA-b, and siRNA-e); and (v) four input cues (a, c, b, and e) that each regulate one or two mediators. FIG. 5E depicts a molecular AND circuit, which contains: (i) an output protein (DsRed); (ii) two identical regulatory proteins, whose encoding mRNAs each independently contain multiple response elements (e.g., Target-a, Target-c, or Target-e, or Target-NOT(a) and Target-b, respectively) for different siRNA mediators; (iii) five siRNA mediators (siRNA-a, siRNA-NOT(a), siRNA-c, siRNA-b, and siRNA-e); and (iv) four input cues (a, c, b, and e) that each regulate one or two mediators.

FIG. 6 depicts an exemplary circuit for use in detection of miRNA expression patterns.

FIG. 7 depicts an exemplary dual circuit design comprising a pattern of miRNA mediators and that can evaluate logic OR combinations of endogenous miRNAs.

FIG. 8A depicts an exemplary AND gate circuit implementation. The circuit comprises: (i) a pair of input cues (A) and (B); (ii) a pair of siRNA mediators—siRNA-A and siRNA-B—that are negatively regulated by input cues (A) and (B), respectively; and (iii) an output protein (ZsYellow), the mRNA which encodes the output protein being regulated by the siRNAs via two siRNA target sites (Target-A and Target-B). FIG. 8B depicts a circuit comprising: (i) a pair of input cues (A) and (B); (ii) a pair of miRNA mediators—miRNA-A and miRNA-B—that are negatively regulated by input cues (A) and (B), respectively; and (iii) an output protein (ZsYellow), the mRNA which encodes the output protein being regulated by the miRNAs via two miRNA target sites (Target-A and Target-B). FIG. 8C depicts a molecular circuit of FIG. 8B, wherein the input cues A and B are exemplified by Endogenous transcription factors A and B, respectively. FIG. 8D depicts how the Endogenous transcription factors A and/or B, can regulate the expression of their corresponding miRNAs.

FIG. 9A-9C depict a logic table and exemplary circuits for use in multi-color detection of multiple conditions. FIG. 9A depicts how three different conditions are defined using three input signals. Each condition is represented by a DNF logic expression. Note that the conditions are non-overlapping in a sense that every logic AND clause appears only in one condition. Combinations of Cyan and Red proteins encode the presence of the various conditions as shown in the table. FIG. 9B depicts the design of the computation module. In general, conditions that require single outputs are constructed according to the standard architecture, with the required fluorescent output placed ahead of small RNA target sites (indicated only by the names of their corresponding logic variables). Conditions that require multiple outputs (such as A and B and C that requires Cyan and Red) are tested by two parallel constructs that have the same set of small RNA targets but encode both output proteins. All these constructs are placed together in the cells or organisms we wish to probe; in other words the networks in FIG. 9B are a single circuit. FIG. 9C depicts a sensory module of the circuit in Fig. B. Mediator small RNAs (indicated as M-A, etc.) are required to represent all variables and their negation. The regulatory links from the input are exactly as in the standard DNF architecture.

FIG. 10 is a series of photographs of fluorescence images of cultured cells, which depict the crosstalk verification between siRNAs and their targets. Each row represents a construct treated with different siRNAs (columns). In an experiment, 100 ng of a construct, 100 ng of the pAmCyan-C1 transfection control and 10 μmol of siRNA were transiently cotransfected into 293-H cells and assayed after 48 hours. The images combine the fluorescent signal from the amCyan transfection control and the signal from the zsYellow protein expressed from the clause molecules. Low levels of ZsYellow result in red images while coexpression of both proteins results in mostly green and yellow spots. Negative control is a nonsense siRNA provided in the same amount as the active siRNAs. The bar charts to the right of each row show the relative intensity of the reporter protein ZsYellow as measured by FACS and normalized to the negative control samples.

FIG. 11 is a series of exemplary nucleic acid sequences and a series of photographs of fluorescence images of cultured cells, which depict the testing individual DNF clause molecules. The constructs and their common feature (top-left) are shown to the left. 10 μmol of each indicated siRNAs (columns) were cotransfected with 100 ng of the indicated clause molecule (rows) and 100 ng of the transfection control plasmid pAmCyan-C1 into 293-H cells and assayed after 48 hours. The images combine the fluorescent signal from the amCyan transfection control and the signal from the zsYellow protein expressed from the clause molecules. Low levels of ZsYellow result in red images while coexpression of both proteins results in mostly green and yellow spots. Negative control is a nonsense siRNA provided in the same amount as the active siRNAs. The quantitative results that correspond to the images, obtained by FACS measurements and normalized to the negative control for each construct, are shown on the right. The straight black lines appearing beneath the sequences in the figure demarcate the individual target subsequences in each larger sequence. The following sequences are depicted:

(SEQ ID NO: 49)

CTTAACAAGCTTCGACACGTACGCGGAATACTTCGAAAGCGTTGCTAGTA
CCAACCCTAACGGCAAGCTGACCCTGAAGTT;

(SEQ ID NO: 50)

CTTAACAAGCTTCGAAACGATATGGGCTGAATACAAAAGCGTTGCTAGTA
CCAACCCTA;

(SEQ ID NO: 51)

CTTAACAAGCTTCGACCGCTTGAAGTCTTTAATTAAACACGTACGCGGAA
TACTTCGAAACGGCAAGCTGACCCTGAAGTT;
and

(SEQ ID NO: 52)

CTTAACAAGCTTCGAAACGATATGGGCTGAATACAAACCGCTTGAAGTCT
TTAATTAAA.

FIGS. 12A-C are a series of two-dimensional structures of nucleic acid sequences and a bar graph depicting secondary structures and folding energies of the target permutation in the clause sequence T1-S14-FF4, as predicted by mfold software from rpi.edu. The folding was done at 1 M NaCl, with no divalent ions at 37° C. The folded sequences comprise the 3′-UTR inserts and contain a stop codon, a short spacer and siRNA target sites, along with an extra G nucleotide. The bar chart shows the relative downregulation measured with three different variants by cotransfecting 293-H cells with 2.5 pmol of each siRNA with 100 ng of the constructs and 100 ng of AmCyan-C1 control plasmid and assaying after 48 hours. The experimentally tested variants are shaded to indicate the siRNA target sequences as in FIG. 11. The following sequences are depicted:

(SEQ ID NO: 53)

UCGAGCUUAACAAGCUUCGACACGUACGCGGAAUACUUCGAAACGGCAAG
CUGACCCUGAAGUUCCGCUUGAAGUCUUUAAUUAAAG;

(SEQ ID NO: 54)

UCGAGCUUAACAAGCUUCGAACGGCAAGCUGACCCUGAAGUUCCGCUUGA
AGUCUUUAAUUAAACACGUACGCGGAAUACUUCGAAG;

(SEQ ID NO: 55)

UCGAGCUUAACAAGCUUCGACCGCUUGAAGUCUUUAAUUAAAACGGCAAG
CUGACCCUGAAGUUCACGUACGCGGAAUACUUCGAAG;

(SEQ ID NO: 56)

UCGAGCUUAACAAGCUUCGAACGGCAAGCUGACCCUGAAGUUCACGUACG
CGGAAUACUUCGAACCGCUUGAAGUCUUUAAUUAAAG;

(SEQ ID NO: 57)

UCGAGCUUAACAAGCUUCGACACGUACGCGGAAUACUUCGAACCGCUUGA
AGUCUUUAAUUAAAACGGCAAGCUGACCCUGAAGUUG;
and

(SEQ ID NO: 58)

UCGAGCUUAACAAGCUUCGACCGCUUGAAGUCUUUAAUUAAACACGUACG
CGGAAUACUUCGAAACGGCAAGCUGACCCUGAAGUUG.

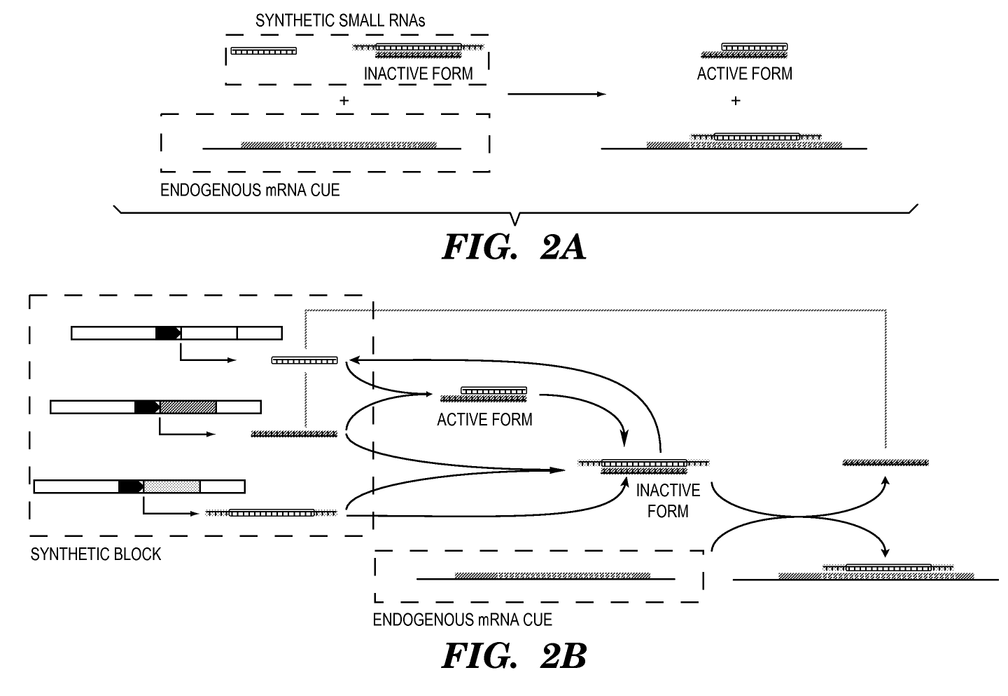

FIGS. 13A-13D depict an operation of the Boolean evaluator. (FIG. 13A) Two expressions in DNF form are evaluated for all possible variable assignments as indicated in the figure. 2.5 pmol of each input siRNA (or 2.5 pmol of the Negative Control siRNA in the case of an absent input siRNA) were cotransfected with 100 ng of each clause molecule and 100 ng of the AmCyan transfection control into 293-H cells and assayed after 48 hours. The quantitative results corresponding to the images, obtained using FACS are shown on the right (see Methods) (FIG. 13B). The histogram of the expression levels obtained in D1 and D2 evaluations, showing the number of evaluation experiments whose levels of expression are grouped together. (FIG. 13C) An evaluation of two CNF expressions. In C1 evaluation experiments using LacI, 10 pmol of each siRNA, 50 ng of the CMV-LacI-FF3-FF4 clause molecule, 200 ng of CAGOP-dsRed-monomer reporter and 100 ng of pAmCyan-C1 transfection control were cotransfected into 293-H cells and assayed after 48 hours. The expression levels of the reporter obtained by FACS are given relative to the control experiments where active siRNA was replaced with the same level of nonsense siRNA (first row of images). In C1 evaluation experiments using LacI-KRAB, 5 pmol of each siRNA, 5 ng of the CMV-LacI-KRAB-FF3x3-FF4x3 clause molecule, 200 ng of CAGOP-dsRed-monomer reporter and 100 ng of pAmCyan-C1 transfection control were cotransfected into 293-H cells and imaged after 48 hours. The expression levels of the reporter given in the figure were obtained by FACS using 100 ng of pZsYellow-C1 transfection control instead of pAmCyan-C1 and they are given relative to the control experiments where active siRNA was replaced with the same level of nonsense siRNA (first row of images). In C2 evaluation experiments, 5 pmol of each siRNA, 50 ng of CMV-LacI-FF3x3 and CMV-LacI-FF4x3 clause molecules, 200 ng of CAGOP-dsRed-monomer reporter and 100 ng of pAmCyan-C1 transfection control plasmids were cotransfected into 293-H cells and assayed after 48 hours. It was quantified similarly to the C1 experiments with LacI. (FIG. 13D) A demonstration of anticorrelated evaluation results provided by two circuits operating in parallel. 10 pmol of siRNA (or nonsense siRNA), 100 ng of pZsYellow-F3x3 and 50 ng of CMV-LacI-F3x3 clause molecules and 200 ng of CAGOP-dsRed-monomer reporter were cotransfected into 293-H cells and assayed after 48 hours. Each reporter (ZsYellow and dsRed) was quantified independently (see Methods) and given relative to their respective “True” expression levels.

FIGS. 14A-14E are (14A): a generic motif topology for stabilizing the expression of network elements regulated by miRNAs; (14B) transcriptional repressors (the line drawn between Gene 1 and Gene 2 represent their coupled expression from a single bidirectional promoter); (14C): a repressor and an miRNA; (14D): an exemplary molecular implementation of the motif depicted in 14A; and (14E): an exemplary molecular implementation of the motif depicted in 14B.

FIGS. 15A and 15B is (15A): an exemplary molecular circuit comprising a “noise-reduction mechanism;” and (15B) a histogram depicting the results of a flow cytometry analysis illustrating that the output of the measured dsRed levels produces a narrow (thus reduced fluctuation) output of fluorescent in comparison to the negative control. The Y-axis represents the number of cells and the X-axis represents the intensity of the fluorescence signal produced by the dsRed protein. “Motif” represents cells in which the noise-reduction mechanism of the circuit is active. The negative control is the circuit with the addition of IPTG which inhibits the function of LacI and thus inactivates the noise-reduction mechanism.

FIGS. 16A-16F are (16A): a histogram depicting a molecular band-pass filter response to an input; (16B): an exemplary circuit structure to implement molecular band-pass filter behaviour with biological molecules; (16C) an exemplary circuit comprising a transcriptional activator and a small hairpin RNA repressor; (16D) a bar graph depicting the experimental results in cells using the exemplary circuit of FIG. 16C (the copy number of the cassette CN1 was systematically increased (x axis) and the DsRed output measured in cell culture (y axis)); (16E) an exemplary circuit with a transcriptional activator and a transcriptional repressor; and (16F) an exemplary circuit that generates a molecular band-pass response with respect to an input transcriptional activator (TF).

### DETAILED DESCRIPTION

Disclosed herein are autonomous molecular circuits that can function in cells. The circuits can process logical operations in which one or more input cues are among the operands and produce an appropriate output. Such circuits can be implemented in living cells, e.g., eukaryotic or prokaryotic cells that have been modified to include circuit components. Molecular circuits and cells can be used in a variety of applications, for example, diagnostics, therapeutics, and protein production.

In one aspect, this disclosure provides circuit components, which can be used to implement any arbitrary Boolean operation in living cells. Individual components for particular operations can be coupled to inputs and to one another in order to implement a circuit that operates on a complex expression.

An automaton decision process can be based on such cues as inputs. The circuit may process a Boolean expression that connects logic variables representing the cues via logic operations. A decision is made by assigning truth values to the variables (TRUE when the cue is present and FALSE otherwise) and evaluating the expression. TRUE and FALSE expression values would indicate, respectively, positive and negative decisions.

All Boolean expressions can be converted to a standard form, e.g., either of two standard forms (Disjunctive Normal Form, DNF, and Conjunctive Normal Form, CNF) that use only AND, OR, and NOT operations, with the latter applied to individual variables only. Accordingly, in many implementations, the key circuit components include an AND operator (an “AND circuit”), an OR operator (an “OR circuit”), and a NOT operator. A reduced set of operators simplifies the toolbox needed for implementing any Boolean expression. For example, any arbitrary Boolean expression can be reduced to a normal form (e.g., disjunctive or conjunctive normal form).

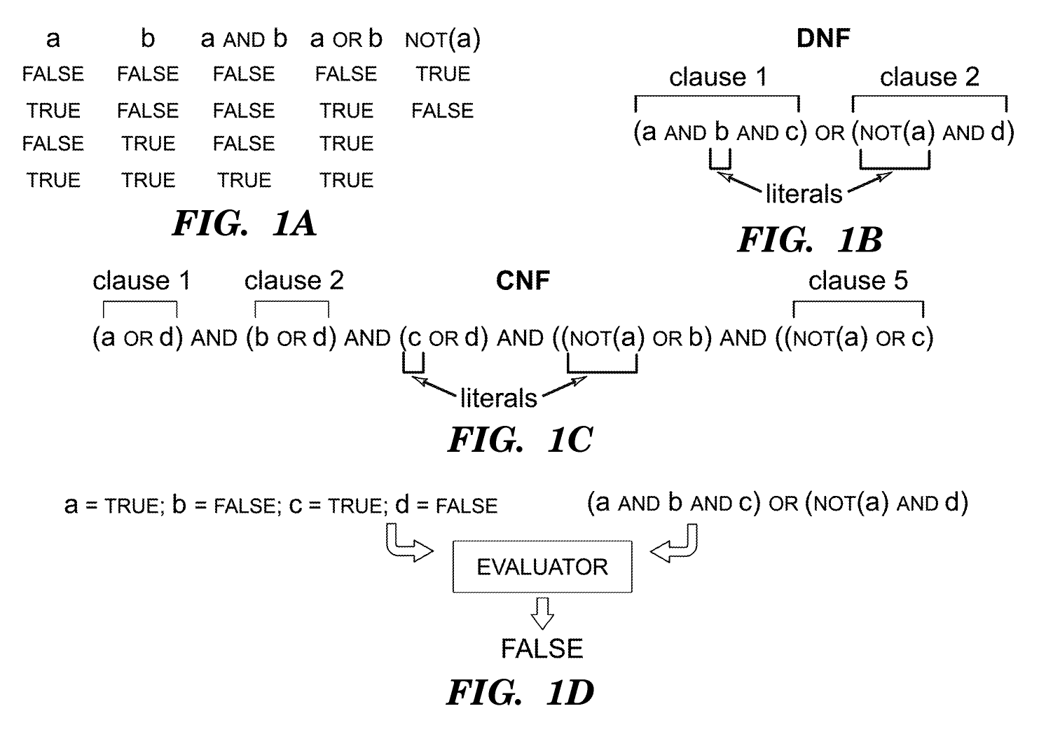

A variable and its negation are called, respectively, positive and negative literals. Standard expressions are constructed from sub-expressions called clauses. Within each clause, one type of operation is applied to the literals (AND in DNF, OR in CNF). The clauses are connected by the complementary operations: OR in DNF, AND in CNF (FIG. 1A-1D).

Once an expression has been reduced to a normal form, circuit components are selected according the Boolean operators in the normalized expression. Such components can then be introduced into one or more cells in order implement the circuit in the cells. By reducing expressions to a normal form, a basic set of circuit components (e.g., AND/OR/NOT) can be used to implement more complex operators such as XOR, NAND, NOR, or XNOR. Aspects of this implementation are illustrated in FIG. 1A-1D.

Other methods can also be used to parse and implement Boolean expressions. Circuits can be implemented that process at least two, three, four, five, six, seven, eight, nine, or ten Boolean operations. They can be engineered to implement any arbitrary expression. In particular embodiments, the circuits will represent between 1-12, 1-10, 2-6, 3-6, 3-4, or 2-4 literals.

As described herein, the circuit components can be engineered using artificial sequences that are unique within the circuit and sufficiently distinct from endogenous sequences that are expressed (e.g., in the case of mRNA) or that are accessible (e.g., in the case of transcriptional regulatory sites in chromatin). Because of the large number of available artificial sequences, circuits are scalable and complexity and size are not severely limited by the number of circuit components.

### An Exemplary Architecture

A general circuit architecture can include one or more sensor modules that comprise mediators that can detect and respond to input cues. The sensor modules are then integrated using “circuit operators” that implement Boolean operations. The circuit operators control one or more outputs. The output of one operation can serve as the input cue for another operation. (Some implementations, however, can depart from this general architecture. For example, mediators can themselves implement aspects of Boolean operations themselves).

Examples of mediators for the sensor modules include small RNAs and proteins. The mediators can detect intracellular or extracellular parameters. For example, the mediators can be allosterically controlled. Mediators can control the activity of a central circuit operator, e.g., an mRNA encoding an output protein.

Example of circuit operators include mRNAs. For example, an mRNA that includes a plurality of response elements (e.g., in its 3′ UTR) can be used to implement an OR circuit. The use of two alternative promoter constructs, either of which can produce an output protein (or functionally equivalent versions of such a protein) also implements an OR circuit. The production of two proteins whose function is conjunctively needed for activity can be used to implement an AND circuit. The proteins can form a heterodimer, for example, such as Fos-Jun, or can connect a DNA binding domain to a transcriptional regulatory domain (e.g., a transcription activation domain or a transcription repression domain).

To implement complex expression, different circuit operators can be combined together. For example, the output protein for one circuit operator can provide the mediator or other input cue for another circuit operator. In this way, the toolbox of circuit operators described herein can be used to implement any arbitrary Boolean expression.

### Exemplary Sensor Modules and Mediators

Circuit mediators can be used to couple one or more input cues to an output, e.g., according to the Boolean operation that is being implemented. Examples of circuit mediators include, e.g., small interfering RNA (siRNA), short hairpin RNA (shRNA), microRNA (miRNA), ribozymes (RZs), or proteins (e.g., DNA binding proteins (e.g., transcription factors) or RNA binding proteins (e.g., translation elongation or initiation factors such as a ribosome)). A molecular circuit can contain, e.g., at least one, two, three, four, five, or six mediators, e.g., between two and twelve, two and ten, or five and ten mediators.

Mediators can be designed to interface with a circuit operator in order to modulate an output. For example, mediators can act upon a response element in an output (e.g., where the output is an mRNA), or where the output is a protein, the mediator can act upon a response element in the mRNA that encodes the output protein. The response element can be within the coding region of an mRNA, or can be in a non-coding region. For example, the response element can be within the 3′ untranslated region (UTR) or the 5′UTR of an mRNA. An mRNA can have exactly one or more than one response element for a single mediator. An mRNA can also have multiple response elements, each response element acted upon by a unique mediator, e.g., a unique siRNA mediator.

Input cues can be, for example, physical or chemical. Chemical input cues include, e.g., small molecules, alcohols, a biological molecule (e.g., a steroid, a protein, an RNA (e.g., an mRNA; an unspliced, precursor form of an mRNA; or a miRNA), or a metal (e.g., lead, gold, iron, arsenic, beryllium, cadmium, or mercury). Physical input cues include, e.g., radiation, high or low temperatures, or atmospheric conditions (e.g., pressure or oxygen or carbon dioxide tension). An input cue can be the presence or the absence of a chemical or a physical cue. For example, removal or withdrawal of a chemical or physical cue can serve as an input cue for a molecular circuit. A molecular circuit herein can be coupled to, and regulated by, both one or more chemical and one or more physical input cues.

In some embodiments, the circuits described herein can evaluate more than one (e.g., two, three, four, five, six, seven, eight, nine, or 10 or more) input cues in parallel.

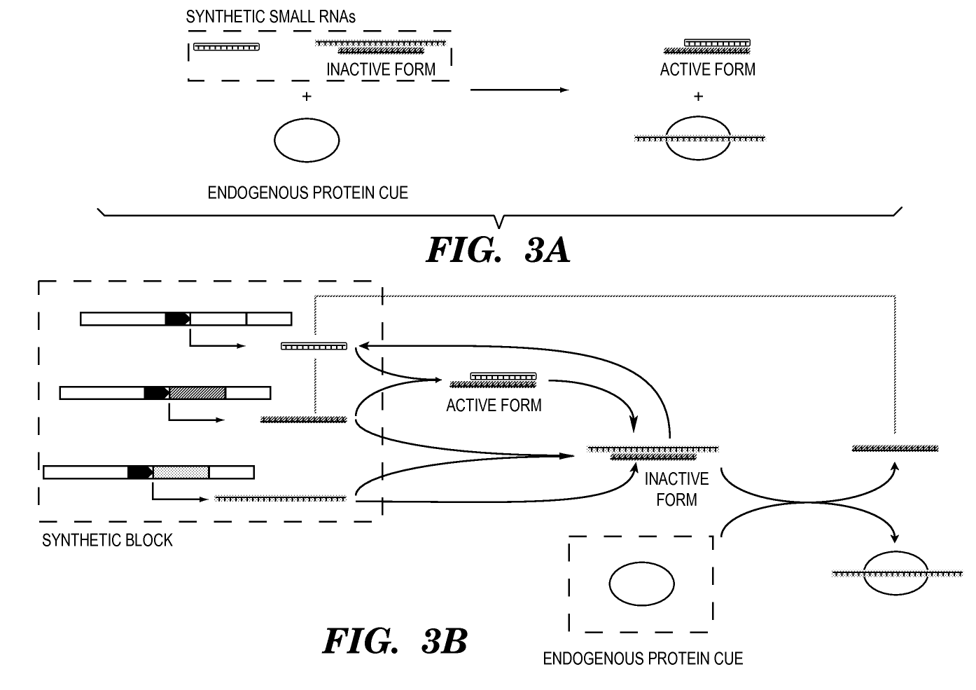

Examples of how a mediator can respond to an endogenous cue are shown in FIGS. 2 (FIGS. 2A and 2B) and 3 (FIGS. 3A and 3B).

FIGS. 2A and 2B depict a mechanism by which an siRNA mediator can respond to an endogenous mRNA cue. The circuit comprises (and encodes) a first siRNA component and a second siRNA component, wherein the association of the first and second siRNA components forms an active siRNA specific for a cellular target. The circuit also comprises (and encodes) an aptamer that binds to the first siRNA component and prevents the formation of the active siRNA (FIG. 2B, “Synthetic Block”). However, in the presence of an mRNA cue, the mRNA specifically binds to the aptamer, thereby allowing the formation of the active siRNA (FIG. 2A).

FIGS. 3A and 3B depict a mechanism by which an siRNA mediator can respond to an endogenous protein cue. As in FIG. 2, the circuit comprises and encodes (i) a first siRNA component and second siRNA component, wherein the association of the first and second siRNA components forms an active siRNA and (ii) an aptamer that binds to the first siRNA component, wherein the binding of the aptamer to the first siRNA component to the aptamer prevents the formation of the active siRNA (FIG. 3B, “Synthetic Block”). In this example, in the presence of a protein cue, the protein specifically binds to the aptamer, thereby allowing the first siRNA to form the active siRNA with the second siRNA (FIG. 3A).

Proteins can be detected in a variety of ways. For example, it is possible to engineer nucleic acid aptamers to specifically bind to a protein or other target. See, e.g., US 2005-0026178 and references describing Systematic Evolution of Ligands by Exponential Enrichment, (SELEX™) a method for making a nucleic acid ligand for any desired target, as described, e.g., in U.S. Pat. Nos. 5,475,096 and 5,270,163.

In some embodiments, a single stranded aptamer is incorporated as loops into a shRNA-like structures, e.g., within a loop. Binding of a protein to the loop moiety of such a shRNA will modulate with its processing by the RNAi pathway, and accordingly provide circuit regulation.

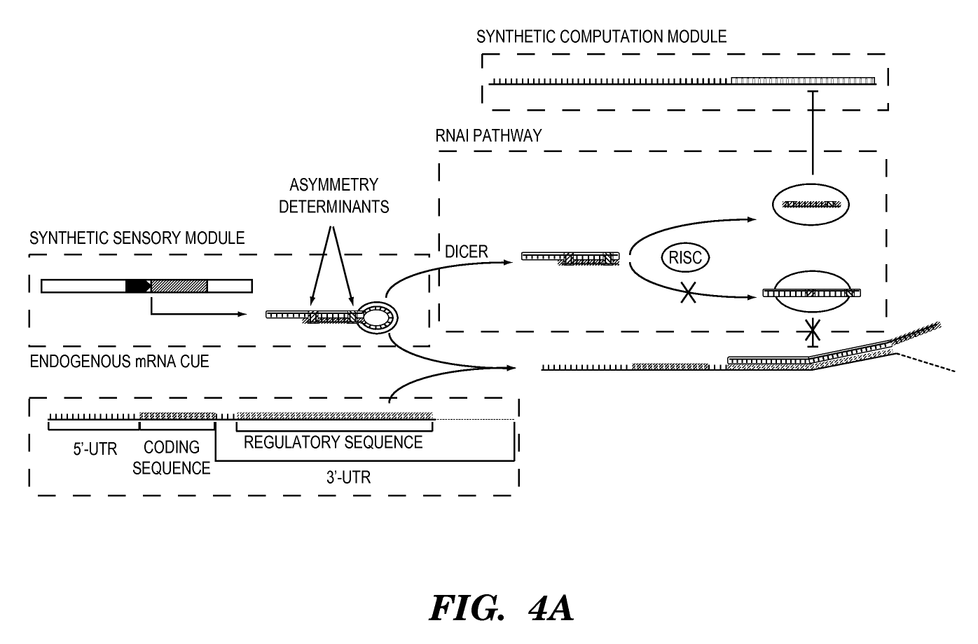

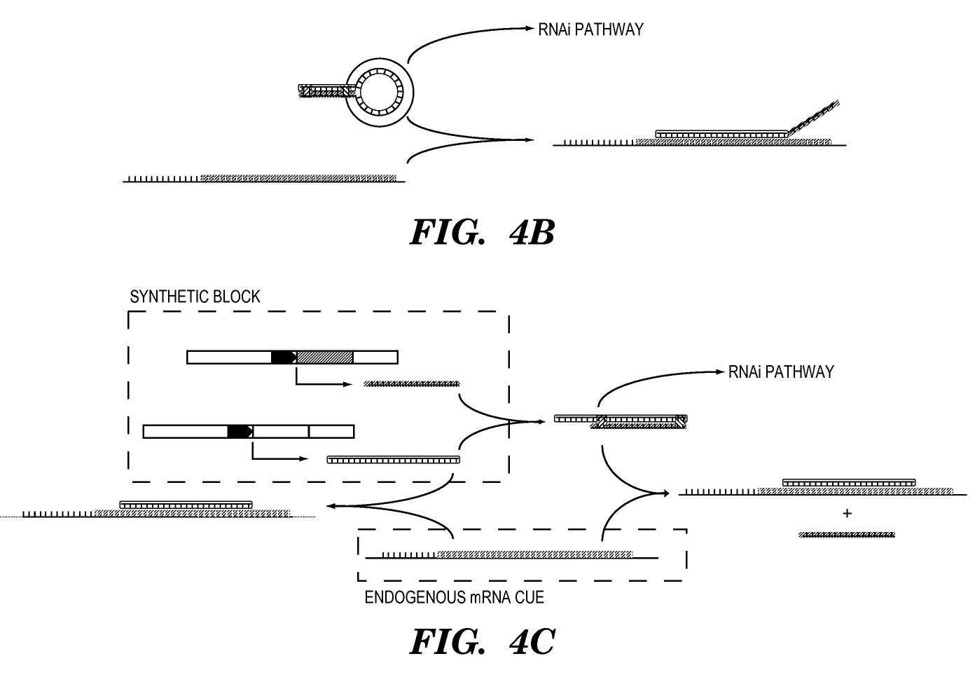

Further examples of mediators that use downregulation of small interfering RNAs and their derivatives are shown in FIG. 4. In general, these mechanisms are based on physical disruption of the shRNA or siRNA via RNA-RNA interactions. Generally, shRNAs and siRNAs are modified with molecular motifs without major modulation of their capability to partake in the RNAi pathway. These inserted non-canonical motifs render shRNAs and siRNAs responsive to eternal molecular cues. The details of exemplary interactions are given in the legend.

Proposed mechanisms of downregulation of siRNA derivatives by an endogenous mRNA are depicted in FIG. 4A. A synthetic shRNA sensor is expressed in situ from a suitable vector. This shRNA variant contains a single-stranded overhang of approximately 10 nucleotides. The overhang, the sense strand of the stem part, and the loop (light shade) are designed to be complementary to a regulatory subsequence of an endogenous mRNA molecular cue (dark shade) located in the 3′ UTR. Although these overhangs are normally not the part of the canonical shRNA transcripts, they may not interfere with the shRNA activity. The shRNA and the mRNA first interact by hybridization of the overhang to the regulatory subsequence and subsequently by strand migration. This interaction results in the unwinding of the shRNA structure and its removal from the normal RNAi pathway. The complementarity between the shRNA and the mRNA is incomplete in order not to trigger down-regulation of the mRNA cue itself, but it is strong enough to afford the required conformational change. The attack on the endogenous cue by the shRNA through the RNAi mechanism is achieved by incorporating asymmetry structural motifs (Schwartz et al. (2003) Cell 115: 199-208) within the shRNA that will preferentially incorporate the antisense (light shade) but not the sense (dark shade) sequence as an RNA guide into the RISC holoenzyme.

FIG. 4B depicts a modification of the above mechanism that relies on the initial hybridization in the loop region rather than 5′-overhang. A sufficiently large loop structure can serve as a good initiator of the hairpin unfolding process. At the same time, a large loop in itself will not prevent the hairpin from participating in the RNAi pathway. FIG. 4C depicts the inactivation of a double-stranded siRNA structure. siRNA may be produced in a cell via a synthesis of separate strands and their annealing in situ (Miyagishi M et al. (2002) Nat. Biotechnol. 20(5):497-500). There are two points of interference with this process. The sense strand of the siRNA may bind directly to the regulatory sequence of the mRNA cue; in addition an annealed duplex siRNA is designed to contain a single-stranded overhang that will still be able to nucleate with the regulatory sequence of the mRNA cue and be subsequently removed via strand migration. This non-canonical dsRNA structure can be active since the helicase activity of the RISC requires an almost blunt end on the 3′-terminus of the duplex but may tolerate 5′-protrusions on the 5′-end.

In addition to the sensor modules described herein it is possible to use molecules described in Isaacs, F. J. et al. Nat. Biotechnol. 22, 841-847 (2004); Penchovsky. R., Breaker, R. R. Nat. Biotechnol. 23, 1424-1433 (2005); Bayer and Smolke (2005) Nature Biotechnol., 23:337-343; US 2007-0089196; and US 2006-0088864. For example, sensor modules can include riboswitches which are complex folded RNA sequences including an aptamer domain for a specific ligand. Riboswitches that act in a “cis” fashion (i.e., that control expression of an operably linked sequence) are known to occur in the non-coding regions of mRNAs in prokaryotes, where they control gene expression by harnessing allosteric structural changes caused by ligand binding. For a review of “cis” riboswitches, see Mandal and Breaker (2004) Nature Rev. Mol. Cell Biol., 5:451-463. Riboswitches that act in a “trans” fashion (i.e., that control expression of a sequence not operably linked to the riboswitch) have also been designed, see, for example, US 2006-0088864.

### Exemplary Circuit Implementations

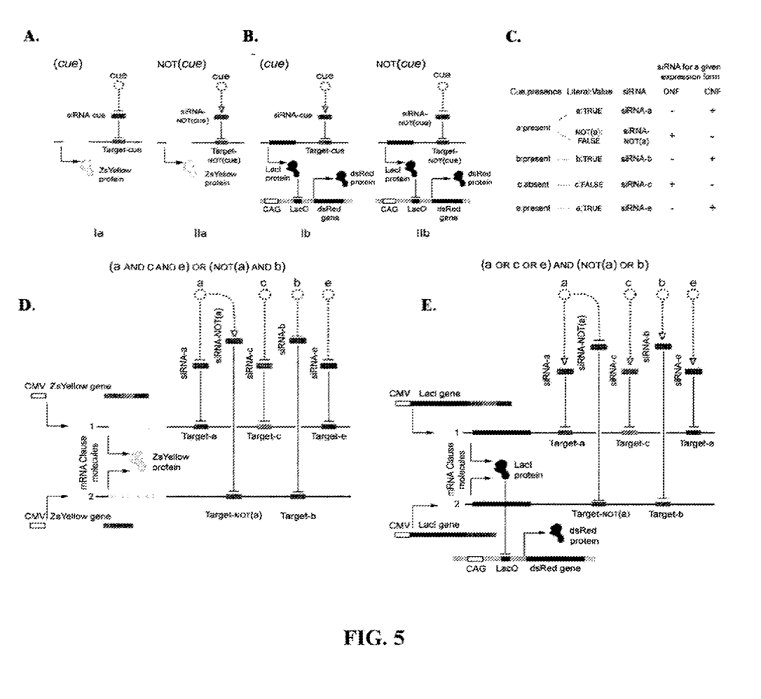

FIG. 5A depicts exemplary molecular circuits having (i) a single output protein (ZsYellow protein); (ii) an mRNA encoding the protein, which mRNA contains one response element for a single siRNA mediator; (iii) an siRNA mediator, and (iv) an input cue that regulates (negatively (FIG. 5A, left) or positively (FIG. 5A, right)) the mediator. Thus, in the presence of the input cue that negatively regulates the siRNA mediator (“(cue)”), the siRNA mediator is inhibited, the mRNA encoding ZsYellow is not degraded and thus, the ZsYellow protein is expressed (see FIG. 5A left). In contrast, in the absence of an input cue that negatively regulates the siRNA mediator (“NOT(cue)”), the mRNA encoding ZsYellow is degraded by the siRNA and thus, no ZsYellow protein is expressed.

A mediator can regulate an output directly or indirectly. For example, an siRNA mediator can directly regulate an output mRNA (or the output protein encoded by the mRNA), e.g., by binding to and negatively regulating the mRNA through one or more response elements in the mRNA (e.g., a response element in the coding region of the mRNA or in a non-coding region such as a 3′ UTR sequence). A mediator can also indirectly regulate an output mRNA (or the output protein encoded by the mRNA). For example, an siRNA mediator can bind to and negatively regulate an mRNA encoding a regulatory protein (see above), where the regulatory protein controls the expression of the output mRNA or protein. A regulatory protein can be an enzyme such as, but not limited to, a kinase, a phosphatase, a glycotransferase, a nuclease, a polymerase, or phosphodiesterase. A regulatory protein can be, e.g., a transcription factor (e.g., LacI, a STAT, a hormone receptor transcription factor (e.g., estrogen receptor)) that transactivates the expression (e.g., mRNA or protein expression) of an output.

An example of a molecular circuit exhibiting indirect regulation by a mediator is described in FIG. 5B. The exemplary molecular circuit contains: (i) an output protein (ZsRed protein); (ii) an mRNA encoding the output protein, (iii) a regulatory protein (LacI), (iv) an mRNA encoding the regulatory protein, which mRNA contains one response element for a single siRNA mediator; (v) an siRNA mediator, and (vi) an input cue that regulates (negatively (FIG. 5B, right) or positively (FIG. 5B, left)) the mediator. Thus, in the presence of the input cue that positively regulates the siRNA mediator (“(cue)”), the siRNA mediator inhibits the mRNA encoding the regulatory protein, thereby preventing the regulatory protein from promoting the expression of the ZsRed output protein (see FIG. 5B left).

In some embodiments, the molecular circuit can produce two functionally identical outputs (e.g., two identical output proteins such as ZsYellow), that are individually regulated by one or more different mediators and/or input cues. For example, the mRNA encoding one of two identical output proteins can be regulated by a first set of mediators (e.g., siRNA mediators), and the mRNA encoding the second of two identical output proteins can be regulated by a different set of mediators (e.g., siRNA mediators). For example, in FIG. 5D, the depicted molecular OR circuit contains: (i) two identical output proteins (ZsYellow proteins); (ii) two mRNAs, each encoding one of the two output proteins, and each mRNA containing multiple response elements (e.g., Target-a, Target-c, OR Target-e; and Target-NOT(a) AND Target-b, respectively) for different siRNA mediators; (iv) five siRNA mediators (siRNA-a, siRNA-NOT(a), siRNA-c, siRNA-b, and siRNA-e); and (v) four input cues (a, c, b, and e) that each regulate one or two mediators. The Boolean expression that represents the ZsYellow expression (TRUE) scenario is (a AND c AND e) OR(NOT(a) AND b). Thus, in the presence of either input cues “(a) AND (c) AND (e)” or “NOT(a) (i.e., the absence of (a)) and (b),” the molecular circuit will produce the ZsYellow protein (see FIG. 5D left).

In some embodiments, the molecular circuit can contain two outputs (e.g., two output proteins) that, when present together, form an output unit (e.g., an output protein unit). For example, an output protein unit can be a dimer (AB) of two output proteins (A and B), wherein each output protein (or the mRNA that encodes the output protein) can be regulated by one or more mediators (and input cues).

A molecular circuit can contain an output that is indirectly regulated by a regulatory protein. In some embodiments, the molecular circuit can contain two identical regulatory proteins, each independently regulated by one or more different mediators (e.g., siRNA mediators) and input cues. For example, FIG. 5E depicts a molecular AND circuit, which contains: (i) an output protein (DsRed); (ii) two identical regulatory proteins, whose encoding mRNAs each independently contain multiple response elements (e.g., Target-a, Target-c, or Target-e, or Target-NOT(a) and Target-b, respectively) for different siRNA mediators; (iii) five siRNA mediators (siRNA-a, siRNA-NOT(a), siRNA-c, siRNA-b, and siRNA-e); and (iv) four input cues (a, c, b, and e) that each regulate one or two mediators. The Boolean expression that represents the DsRed expression (TRUE) scenario is (a OR c OR e) AND (NOT(a) OR b). Thus, in the presence of the input cues “(a) OR (c) OR (e)” AND “NOT(a) (i.e., the absence of (a)) OR (b),” the molecular circuit will produce the DsRed protein (see FIG. 5E).

In some embodiments, a regulatory protein can be a dimer (protein AB) of two regulatory proteins A and B that when present together, form a regulatory protein unit. In some embodiments, each regulatory protein (or the mRNA encoding each regulatory protein) A or B can be regulated by one or more different mediators (and input cues).

A molecular circuit can produce a “TRUE” or “FALSE” output, e.g., the presence or absence of an output. The molecular circuit can also produce a graduated output. For example, a molecular circuit can operate as a rheostat to generate an output commensurate in level or duration of one or more coupled input cues. Such a graduated output production can be useful in diagnostics and/or therapeutic applications described below.

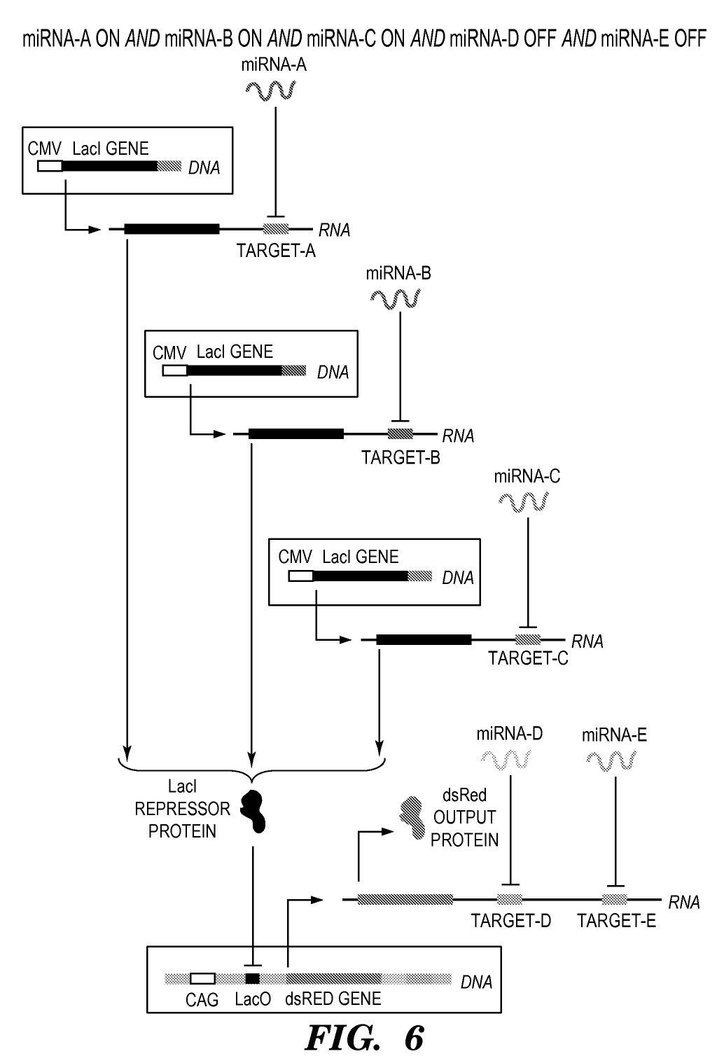

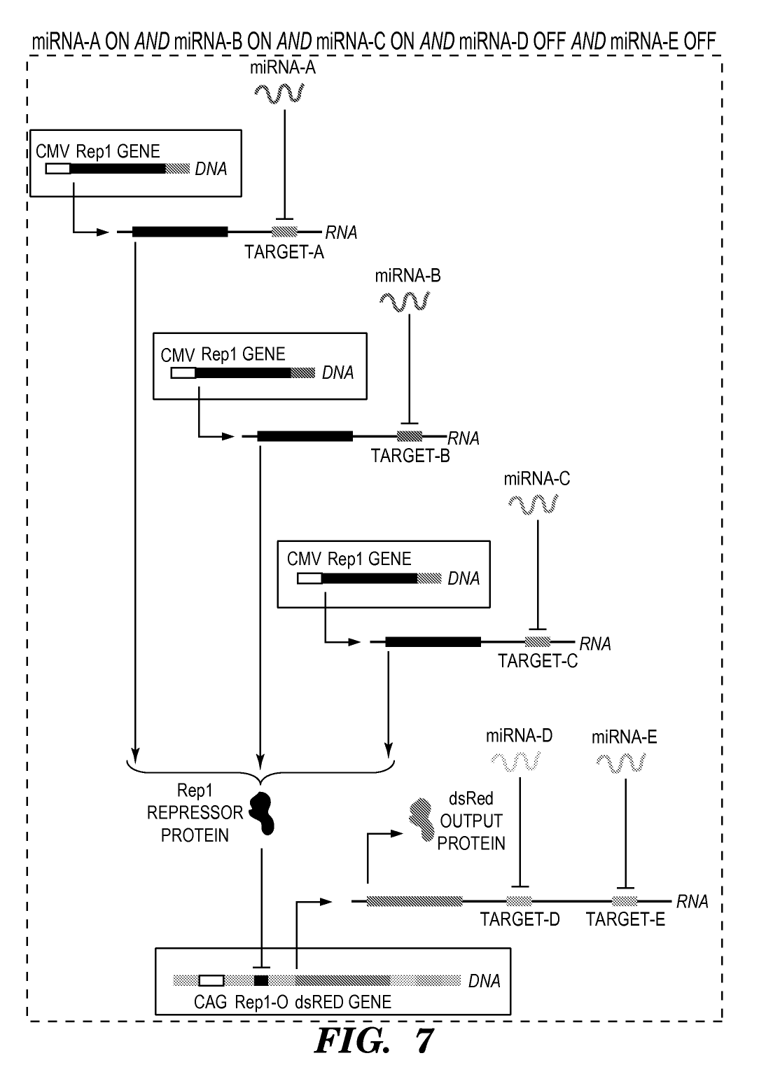

An exemplary circuit design that includes miRNA mediators (wherein the miRNA mediators are also cues) and that can evaluate logic AND combinations of miRNA levels is depicted in FIG. 6. The circuit can contain: (i) a first output protein (LacI repressor protein); (ii) a second output protein (dsRed), which is regulated by the first output protein; and (iii) a pattern of miRNA mediators (miRNA-A, miRNA-B, miRNA-C, miRNA-D, and miRNA-E) whose presence or absence negatively regulates the stability of an mRNA encoding the first output protein or an mRNA encoding the second output protein. Other elements can be either transcribed by the cell from the synthetic constructs or present as endogenous components. The pattern of miRNA mediators can be, e.g., the pattern of miRNAs expressed in a particular cell or tissue type (see below). As depicted in FIG. 6, the targets (Target-A, Target-B, and Target-C) for expressed miRNA-A, miRNA-B, and miRNA-C (respectively) reside in the 3′-UTR of one of three mRNAs encoding the first output protein—the transcription repressor LacI. One target is placed into each UTR. The targets (Target-D and Target-E) for the non-expressed miRNAs are placed sequentially into the 3′-UTR of the mRNA encoding the second output protein dsRed. It follows that in order to detect the expression of the DsRed output in a particular cell, all three of the mRNAs encoding LacI should be inhibited by their corresponding miRNAs and the mRNA encoding the dsRed protein should not be inhibited by its corresponding miRNAs. That is, the cell should express miRNA-A, miRNA-B, and miRNA-C, but not miRNA-D or miRNA-E or dsRed (TRUE) for (miRNA-A AND miRNA-B AND miRNA-C) AND (NOT miRNA-D AND NOT miRNA-E). Such a circuit could be used to, e.g., identify the particular cell from a population of cells (some of which did not satisfy the requirements of dsRed (TRUE).

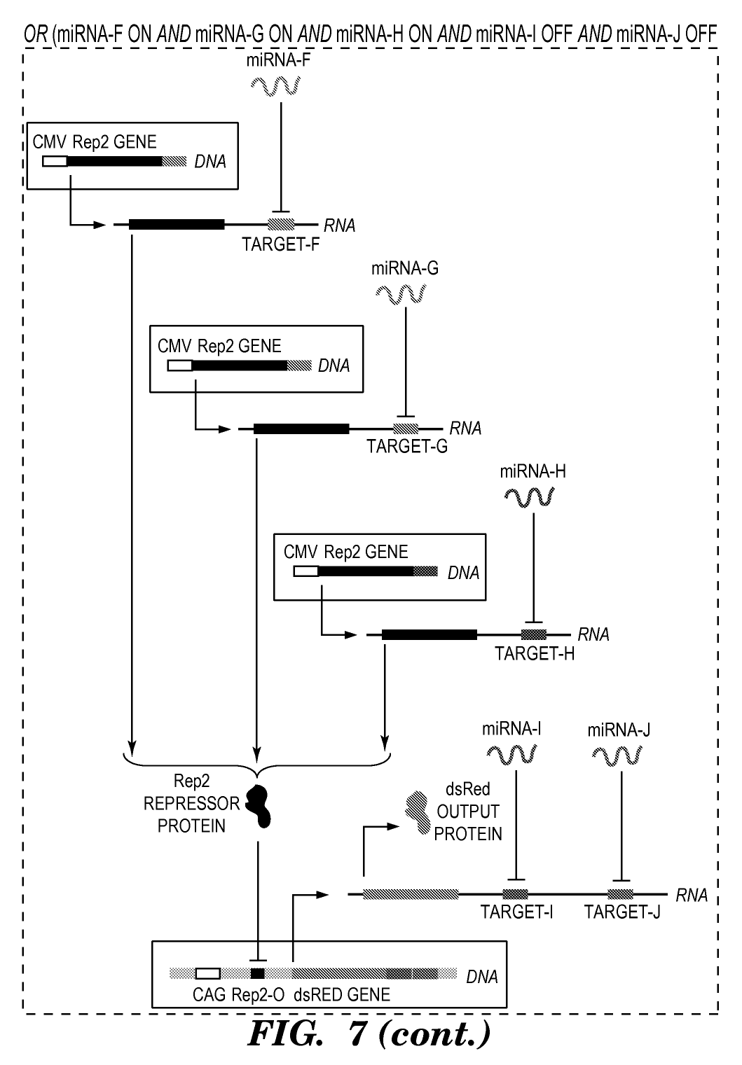

Another exemplary dual circuit design comprising a pattern of miRNA mediators and that can evaluate logic OR combinations of miRNA levels is depicted in FIG. 7. The circuit comprises two subcircuits (one at the left and one at the right of FIG. 7). The first circuit (left) contains: (i) a first output protein (a first repressor protein, Rep 1); (ii) a second output protein (dsRed), which is regulated by Rep1; and (iii) a pattern of miRNA mediators (miRNA-A, miRNA-B, miRNA-C, miRNA-D, and miRNA-E) whose presence or absence negatively regulates the stability of an mRNA encoding Rep1 or an mRNA encoding the dsRed protein. The second circuit (right) contains: (i) a first output protein (a second repressor protein (Rep2)); (ii) a second output protein (dsRed), which is regulated by Rep2; and (iii) a pattern of miRNA mediators (miRNA-F, miRNA-G, miRNA-H, miRNA-I, and miRNA-J) whose presence or absence negatively regulates the stability of an mRNA encoding the Rep2 or an mRNA encoding the dsRed protein. In this dual circuit, dsRed (TRUE) for (miRNA-A AND miRNA-B AND miRNA-C) AND (NOT miRNA-D AND NOT miRNA-E) OR (miRNA-F AND miRNA-G AND miRNA-H) AND (NOT miRNA-I AND NOT miRNA-J).

Such a dual circuit design uses miRNA patterns as a cue for logic computation and can, e.g., (i) detect a single cell or a subpopulation of cells in a larger cell population or tissue or (ii) detect two cell populations in a larger cell population or tissue. For example, the presence of a cell expressing miRNA-A, miRNA-B, miRNA-C, miRNA-F, miRNA-G, and miRNA-H, but not miRNA-D, miRNA-E, miRNA-I, and miRNA-J, can be detected. Alternatively, a cell expressing miRNA-A, miRNA-B, miRNA-C (but not miRNA-D or miRNA-E) and a cell expressing miRNA-F, miRNA-G, and miRNA-H (but not miRNA-I or miRNA-J) could be independently detected or differentiated) in the same population of cells or a tissue.

miRNA-based circuits can also be used to detect logic combinations of transcription factors in live cells. Such implementation takes advantage of the fact that miRNAs are expressed from Pol II promoters. As such, they can be both positively and negatively modulated by endogenous transcription factors. miRNA-based circuits can also work via modulation of miRNA level at the transcription level rather than by direct inhibition of the miRNA itself.

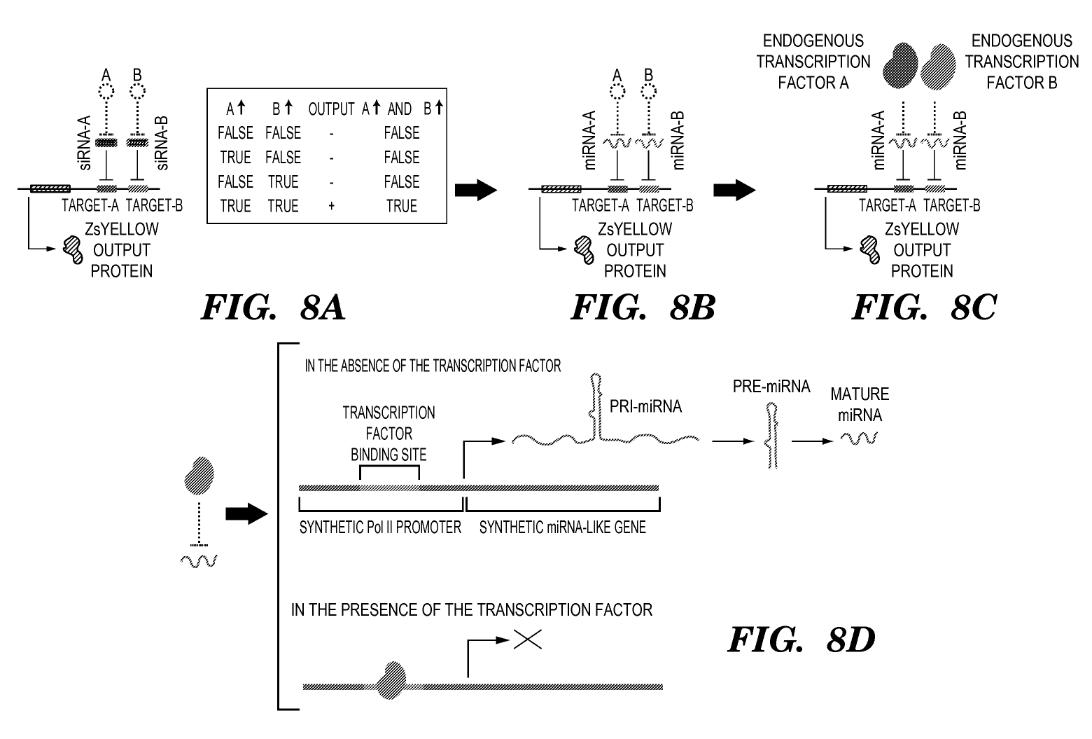

In one implementation, a set of synthetic and exogenous miRNA genes can be designed, and each of these miRNA genes is placed under an artificial Pol II promoter modified to be positively or negatively regulated by an endogenous transcription factor of interest. In this way, logic circuits can be used to detect a pattern of transcription factors, e.g., particular to a cell or subpopulation of cells in a larger cell population, or in a tissue. An exemplary circuit is shown in FIG. 8.

FIG. 8A is an example of an AND gate circuit implementation. The circuit comprises: (i) a pair of input cues (A) and (B); (ii) a pair of siRNA mediators—siRNA-A and siRNA-B—that are negatively regulated by input cues (A) and (B), respectively; and (iii) an output protein (ZsYellow), the mRNA which encodes the output protein being regulated by the siRNAs via two siRNA target sites (Target-A and Target-B). In such a circuit, the presence of both input cues is required for the expression of the ZsYellow. That is, ZsYellow (TRUE) for (A AND B).

The circuit depicted in FIG. 8B comprises: (i) a pair of input cues (A) and (B); (ii) a pair of miRNA mediators—miRNA-A and miRNA-B—that are negatively regulated by input cues (A) and (B), respectively; and (iii) an output protein (ZsYellow), the mRNA which encodes the output protein being regulated by the miRNAs via two miRNA target sites (Target-A and Target-B). As for the circuit depicted in FIG. 8A, the presence of both input cues is required for the expression of the ZsYellow.

FIG. 8C depicts a molecular circuit of FIG. 8B, wherein the input cues A and B are exemplified by Endogenous transcription factors A and B, respectively. FIG. 8D depicts how the Endogenous transcription factors A and/or B, can regulate the expression of their corresponding miRNAs. In this case, a synthetic, exogenous miRNA is expressed from a Pol II-driven construct. A strong constitutive Pol II promoter such as CMV can be modified by incorporation of the transcription factor native binding sites, which can lead to repressed transcription in the presence of the transcription factor. Alternative binding site arrangement could lead to transcription activation in the presence of the factor, thereby affecting positive regulatory link between the factor and the mediator miRNA.

As described above, circuits can be designed and implemented to detect the presence (or absence) of more than one (e.g., two, three, four, five, six, seven, eight, nine, or 10 or more) different conditions (e.g., different cell types, different microbes in a homogenous cell population, different differentiation states of cells in a tissue or population). In some embodiments, this can be done using multi-color coding.

In some embodiments, fluorescent proteins can be used as outputs for the circuits. The number of different fluorescent protein outputs can be, due to spectral overlap, be limited to three different types (cyan, red, and yellow). However, three (or N) colors may be used to detect 2³−1=7 (or, respectively, 2^{N}−1) of different conditions when combinations of colors are used to identify a condition (e.g., condition #1=Cyan ON, Yellow OFF, Red OFF; condition #2=Cyan OFF, Yellow ON, Red OFF, condition #4=Cyan ON, Yellow ON, Red OFF, . . . , condition #7=Cyan ON, Yellow ON, Red ON).

In some embodiments, each condition to be detected using the multi-color coding is mutually exclusive. This reduces complications in interpreting a multi-color output as an overlap of a few single-color outputs.

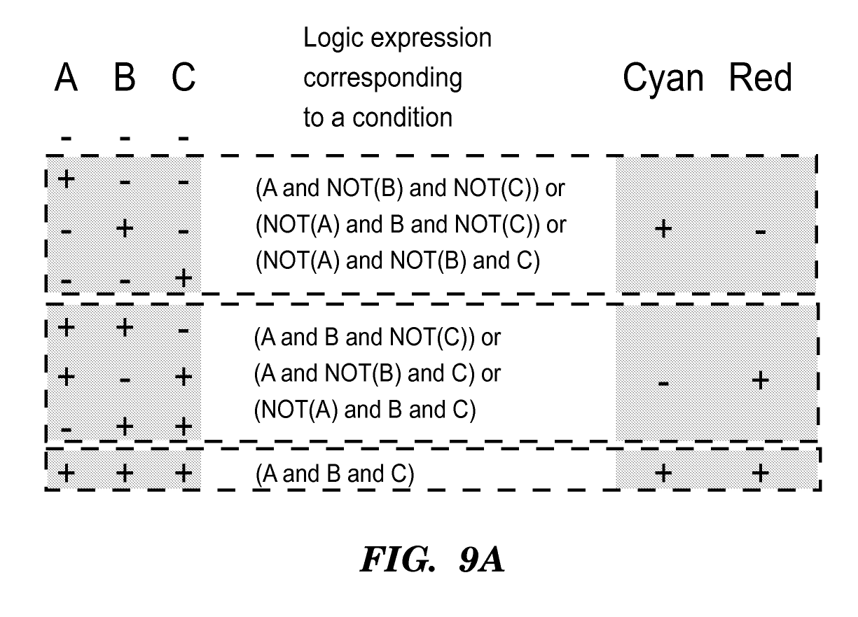

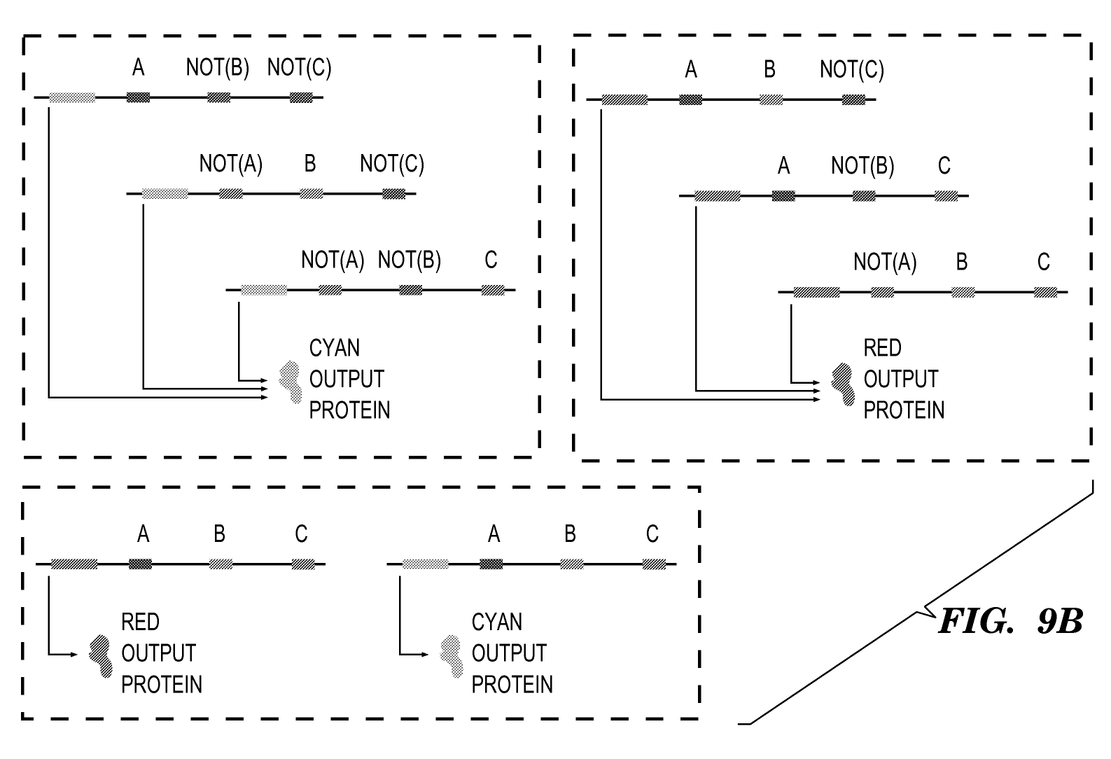

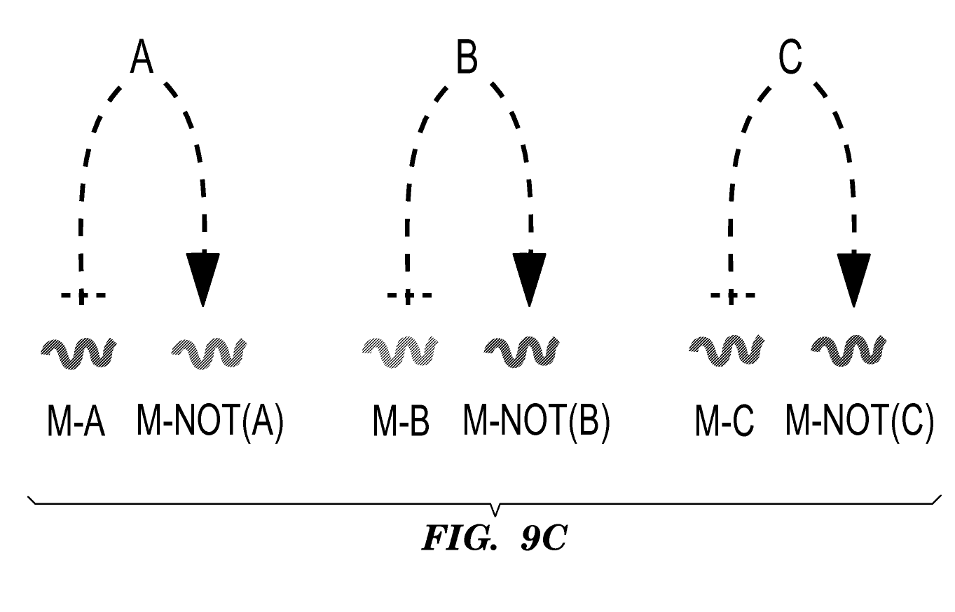

One exemplary circuit design for three input signals and two output colors is shown in FIG. 9. The circuit comprises a “Red” output protein and a “Yellow” output protein, the mRNA encoding each of which containing three regulatory elements A, B, and C. Each of these constructs are placed into a cell, or into each cell in a population of cells. siRNA mediators (indicated as M-A, etc.) represent all variables and their negation.

### Exemplary Outputs

The ultimate output of a molecular circuit described herein can be, e.g., an mRNA, an siRNA (or an shRNA), a protein, or a cellular activity. For example, a protein output can be a reporter such as luciferase, luciferin, green fluorescence protein (GFP), red fluorescence protein (RFP), DsRed, ZsYellow, or an enzyme (e.g., beta-galactosidase, horseradish peroxidase, alkaline phosphatase, or chloramphenicol acetyl transferase (CAT). The output protein can be a selectable marker (e.g., a chemical resistance gene) such as aminoglycoside phosphotransferase (APT) or multidrug resistance protein (MDR). The output protein can also be a pharmaceutical agent (that is an agent with therapeutic ability) or a moiety that triggers the availability of a pharmaceutical agent. The pharmaceutical agent can be, e.g., a small molecule, a protein, or an siRNA (or shRNA).

In some embodiments, the output can alter (e.g., increase or decrease) a cellular activity or cause a cellular event. For example, an output (e.g., an output protein such as a caspase, a death receptor, or a death receptor ligand; or an siRNA (or shRNA) specific for an anti-apoptotic molecule such as Bcl-2 or Bcl-X_{L}) can trigger apoptosis or necrosis of a cell in which the circuit is operating or another cell. The cellular activity can be replication (e.g., increased or decreased replication) of the cell. Replication includes DNA replication as well as cellular duplication. The cellular activity can also be differentiation, migration, or production of a cis- or trans-factor such as a cytokine, chemokine, growth factor, or cell surface receptor.

### Noise-Resistance Mechanisms

Molecular circuits described herein (e.g., diagnostic or therapeutic molecular circuits) can be driven by propagation of one or more input cues (e.g., endogenous disease-related cues) through a series of engineered network elements to affect a specific molecular output(s). To increase the fidelity of this propagation, the molecular circuits can contain one or more elements that decrease or prevent perturbations in the propagation. These elements are referred to as noise reduction mechanisms and they include, e.g., molecular “set-point” circuits and molecular band-pass filters. Perturbations, or “noise,” can be the result of fluctuations in intracellular RNA polymerase activity, cell microenvironment, or the number of gene copies that encode different components of the circuit. For example, a molecular circuit can operate in such a way that an input cue's change from OFF to ON propagates faithfully downstream to inhibit or induce an output, or intermediate, such as an siRNA, mRNA, miRNA, or any other output described herein. This propagation can depend on the absolute and/or relative stoichiometries of any of the circuit components. Random deviations in the concentration of one or more individual components from the desired levels on a timescale that is comparable to, or longer than, that of a molecular computation (the propagation of the one or more cues through the circuit or one or more sub-circuits within a larger circuit) due to extrinsic and intrinsic effects can lead to a deterioration of the computation. For example, an increase in the steady state concentration of one or more circuit components can adversely affect the signal propagation in a negatively regulated circuit.

To minimize these deviations, a self-contained expression unit—a “set-point” circuit—can be constructed for each circuit (or sub-circuit within a larger circuit) that can, e.g., decrease, or prevent, the ON level from exceeding a certain set-point by, e.g., compensating for increase in transcription and translation rates (both mainly affected by increase in the gene copy number, and other factors). Exemplary noise reduction mechanisms for an exemplary circuit are depicted in FIG. 14. (See, e.g., Mangan et al. (2006) J Mol. Biol. 356:1073-1081). In the example shown in FIG. 14, two different strategies are presented for circuit components receiving inputs from small RNAs, and those regulated by transcription factors. These strategies can be combined for components regulated by both small RNAs and transcription factors. FIG. 14 depicts (14A): a generic motif topology for stabilizing the expression of network elements regulated by miRNAs; (14B): transcriptional repressors (the line drawn between Gene 1 and Gene 2 represent their coupled expression from a single bidirectional promoter; and (14C): a repressor and an miRNA (c). FIG. 14D depicts a molecular implementation of the motif in FIG. 14A, wherein each transcript is spliced into a functional mRNA element and an auxiliary miRNA processed from an intron. The miRNA is designed to target the spliced mRNA after both are exported to the cytoplasm, the export being required for proper miRNA processing. This auto-repression is weak enough to maintain enough mRNA to send the ON output signal (a fluorescent protein), but the repression by circuit-related miRNAs (grey color) is strong enough to convert the output to OFF. FIG. 14E depicts a molecular implementation of the motif in FIG. 14B, wherein a strong bidirectional promoter drives expression of a network element and an auxiliary transcriptional repressor. A promoter half that drives the network element also carries a binding site for this auxiliary repressor. The expression from this half is thus negatively adjusted when the baseline promoter's activity increases, reducing the noise in the expression of the network element. As in FIG. 14D, the repression should be moderate to allow for sufficient steady state levels of the network element in an ON state. The element's promoter also carries a DNA binding site for a network-related repressor (grey color) and binding of this molecule leads to strong repression and the OFF output state.

These motifs can compensate for elevated transcription rates, while the set-point can be adjusted by changing interaction parameters as demonstrated by the experiments depicted in FIG. 15.

The aforesaid motifs can be effective in reducing or preventing both extrinsic and intrinsic noise effects that result in random but relatively persistent elevated expression rates. Specifically, the motifs can compensate for increased number of gene copies delivered to cells during introduction (e.g., transfection) of the molecular automata circuits, for example in viral transfections. That is, large scale systems assembled from these components can behave reproducibly in individual cells even if the number of gene copies that code for the individual system's components vary between cells. In addition, these motifs can improve the signal-to-noise ratio of circuits operating in cells, which in essence corresponds to increasing the ratio of useful information to false or irrelevant data. Finally, the motifs can lead to better scalability of the circuits, as reliable elements are easier to combine together to afford large-scale systems.

Any of the molecular circuits described herein can also contain one or more (e.g., two, three, four, five, six, seven, eight, or nine or more) filters or “molecular band-pass filters” (FIGS. 16A-16F). As used herein, a filter is a device (such as a circuit component or sub-circuit within a larger circuit) that generates a response when one or more signals' (e.g., one or more input cues') intensity reaches a certain range of values. Such filters can be adjustable in terms of bandwidth and amplitude. The filters can comprise one or more (e.g., two, three, four, five, six, seven, eight, nine, or 10 or more) filter effectors. One general network structure is shown in FIG. 16B. Two correlated genes (a first gene (gene 1) and a second gene (gene 2)) produce an activator element and a repressor element, respectively. An example of two correlated genes are genes in the same DNA cassette, and regulated by the same promoter. Both of these elements ultimately target the same protein (red). An external input cue can potentially induce both these elements. Thus, in the absence of the input molecule, increasing the dosage of the cassette increases the dosage of genes 1 and 2 simultaneously and leads to an increase, and then a decrease, of the output protein concentration. Exemplary circuits comprising filters are depicted in FIGS. 16C, 16E, and 16F and described in the accompanying Examples. The exemplary circuit depicted in FIG. 16F, e.g., is responsive to a transcription factor input cue.

Further exemplary implementations of noise-reduction mechanisms (such as filters or molecular band-pass filters) as part of any of the molecular circuits described herein are set forth in the accompanying Examples.

### Vectors and Transfected Cells

The molecular circuits described herein can be introduced into cells on a transient or permanent basis. For example, if transient, cells can be transfected with RNA and protein components for operating the circuits. The circuit will expire as the molecules get turned over within the cell. In another transient embodiment, the cells are transfected with one or more DNAs or other nucleic acid encoding the circuit components and the components are produced until the coding DNA is turned over. For more permanent systems, the coding DNAs can be permanently introduced into the genome of the cell.

Accordingly, one or more components of any of the molecular circuits described herein can be encoded by nucleic acids and delivered into cells using nucleic acid vectors. Exemplary methods of constructing such vectors are set forth in the accompanying Examples and are also described in, e.g., Sambrook et al., Molecular Cloning: A Laboratory Manual Second Edition vol. 1, 2 and 3. Cold Spring Harbor Laboratory Press: Cold Spring Harbor, N.Y., USA, November 1989.

Vectors include, e.g., viral vectors, plasmids, and artificial chromosomes. Suitable viral vectors depend, of course, on the type of cell (e.g., bacterial cell, fungal cell, insect cell, or mammalian cell) being infected and can include, e.g., adenovirus, retrovirus (e.g., lentivirus), baculovirus, and phage-based vectors.

In some embodiments, all components of a given molecular circuit can be encoded in a single vector. For example, a retroviral vector can be constructed, which contains all components necessary for a functional molecular circuit described herein. In some embodiments, individual components (e.g., an output, a mediator, or a regulatory protein) can be separately encoded in different vectors and introduced into one or more cells separately. For example, a molecular circuit can be introduced into a cell encoded on multiple vectors, each vector encoding one or more components of an operational molecular circuit.

The molecular circuits described herein can be introduced into a variety of cells including, e.g., fungal, plant, or animal (nematode, insect, plant, bird, reptile, or mammal (e.g., a mouse, rat, rabbit, hamster, gerbil, dog, cat, goat, pig, cow, horse, whale, monkey, or human)). The cells can be primary cells, immortalized cells, or transformed cells. The cells can be those in an animal, e.g., a non-human mammal. Expression vectors for the components of the circuit will generally have a promoter and/or an enhancer suitable for expression in a particular host cell of interest.

A promoter can be constitutive or inducible (conditional). Conditional promoters can be used as a sensor module that feeds into the molecular circuit. Examples of conditional promoters include promoters that are chemically regulated (e.g., a promoter whose transcriptional activity is regulated by the presence or absence of a chemical inducing agent such as an alcohol, tetracycline, a steroid, a metal, or other small molecule) or physically regulated (e.g., a promoter whose transcriptional activity is regulated by the presence or absence of a physical inducer such as light or high or low temperatures).

The cells described herein can be transfected with the circuit components or vectors by any suitable method, such as the methods disclosed by Sambrook et al. (supra). For example, circuit components and vectors can be introduced to the cells by calcium phosphate, electroporation, heat shock, liposomes, or transfection reagents such as FUGENE® or LIPOFECTAMINE®, or by contacting naked nucleic acid vectors with the cells in solution.

The vectors can be stable and permanent, or transient. In some implementations, vectors can be engineered such that they do not replicate in the host cell and therefore are only transiently expressed. An example of such a vector is a plasmid that contains a bacterial origin or replication, but not a eukaryotic origin of replication. Components of a molecular circuit herein can be encoded by integrative vectors or autonomous vectors. In some embodiments, the components of a circuit can be encoded on both integrative and autonomous vectors.

In some embodiments, one or more components of an autonomous molecular circuit are transiently present in the host cell. For example, a molecular circuit can be introduced to the cell as non-integrative vectors, which vectors have a finite stability in the cell (e.g., one or two cell doublings). In some embodiments, one or more (or all) of the components of a molecular circuit can be removed from a cell in which they were stability integrated. For example, a nucleic acid encoding one or more components of a molecular circuit can be conditionally deleted from the host genome using, e.g., a site-specific DNA recombinase such as the Cre-loxP system (see, e.g., Gossen et al. (2002) Ann. Rev. Genetics 36:153-173; U.S. Application Publication No. 20060014264; Fukushige et al. (1996) DNA Research 3(2):73-80, the disclosures of each of which are incorporated by reference in their entirety). Other site-specific recombination methods that can be used to introduce into a cell any of the molecular circuits described herein include, e.g., Flp-In™ Systems (Invitrogen, Carlsbad, Calif.). Such a system allows for the integration of one or more nucleic acid components of the circuit into a mammalian cells at a specific genomic location. (See, e.g., Fujimoto et al. (2006) Genes to Cells 11(5):525-530).

The various nucleic acids encoding components of the molecular circuits described herein can be incorporated into a single plasmid or they can be incorporated into separate vectors, each of which can be introduced into a cell. Where the genes of interest are incorporated into separate vectors, the vectors can be mixed and introduced into the cell together as a single vector sample (e.g., a single plasmid sample). As described above, where the molecular circuit comprises two or more cells, separate nucleic acids encoding different components of the molecular circuit can be introduced into different cells.

Prior to introducing the vectors into a target cell of interest, the vectors can be grown (e.g., amplified) in bacterial cells such as Escherichia coli (E. coli). The vector DNA can be isolated from bacterial cells by any of the methods known in the art which result in the purification of vector DNA from the bacterial milieu. The purified vector DNA can be extracted extensively with phenol, chloroform, and ether, to ensure that no E. coli proteins are present in the plasmid DNA preparation, since these proteins can be toxic to mammalian cells.

The disclosure also features cultures of cells and substantially pure cultures of cells, which cells contain the molecular circuit described herein. In some embodiments, the cultures comprise one or more pluralities of cells, each plurality containing a different autonomous molecular circuit, or different components of an integrated molecular circuit.

The cells containing the autonomous molecular circuits described herein can be stored, for example, as frozen cell suspensions, e.g. in buffer containing a cryoprotectant such as glycerol or sucrose, as lyophilized cells.

A molecular circuit described herein can be implemented in a single cell or can be implemented using multiple cells. For example, a first subset of one or more components can be implemented in one cell, and a second subset of one or more components can be implemented in a second cell. The two cells can communicate, for example, using cell surface signaling molecules (e.g., integrins, selectins, cadherins, or IgSF cell adhesion molecules); by the secretion and detection of a protein ligand (e.g., a cytokine, a growth factor, or a chemokine); or through intercellular gap junctions (connexons). In one embodiment, such circuits are provided in a tissue structure that includes the one or more cells. In some embodiments, multiple circuits can be implemented in a single cell.

In some embodiments, circuits can be implemented in an animal, e.g., a non-human mammal, particularly a transgenic non-human mammal.

### Exemplary Applications

Molecular circuits and cells (e.g., transiently modified cells, transfected cells, or permanently modified cells) containing the circuits have a wide variety of applications, including ones in which the cells are used outside of an organism and one in which the cells are used within an organism, e.g., in a patient or in subjects of veterinary or agriculture applications. Some exemplary applications in which the molecular circuits and cells can be used are detailed in the following sections (and in the Examples).

Diagnostic Applications. The molecular circuits described herein can be used in a number of diagnostic applications. For example, the molecular circuits can be used to detect biomarkers associated with disorders such as, but not limited to, cancer, metabolic disorders, infections, or immunological disorders (e.g., autoimmune diseases).

In some embodiments, the circuits can respond to the presence of a biomarker (e.g., a protein, an mRNA, a small nucleotide polymorphism (SNP), or metabolite) and produce a detectable output. For example, a biopsy of a suspect growth (a growth suspected of being a cancer) in a patient can be performed to obtain one or more cells of the growth. The cells can be transfected with the components of a molecular circuit capable of detecting the presence of a cancer-indicative biomarker input cue ([cancer biomarker] TRUE) and producing a detectable output in response to the input cue.

The circuit can respond to one or more input cues. For example, the circuit can respond to the presence of more than one biomarker. The circuit can also response to the absence of one or more biomarkers. As above, the circuit can respond to a complex Boolean expression such as, e.g., ([biomarker 1] AND [biomarker 2] AND [biomarker 3]) OR (NOT[biomarker 4] OR NOT[biomarker 5] OR [biomarker 6]).

In some embodiments, a molecular circuit described herein can be used to follow differentiation events in real time. For example, a molecular circuit can be introduced into a stem cell and produce one or more outputs indicative of different stages of differentiation, in response to one or more input cues indicative of differentiation state. The various differentiation state input cues and outputs can be encoded/produced by a single, complex molecular circuit or can be encoded/produced by multiple circuits embedded in the cell.

In some embodiments, the molecular circuits described herein can be used as probes, e.g., to guide surgery or detect disease. For example, an area suspected of containing cancer cells (e.g., a primary tumor or microscopic metastases) can be exposed to a vector (or multiple vectors) encoding a molecular circuit capable of detecting one or more cancer-specific biomarkers (cancer-specific biomarker input cues) and producing a detectable protein output (e.g., a fluorescent protein or a enzyme capable of performing a detectable reaction (e.g., (β-galactosidase alkaline phosphatase or horseradish peroxidase). Thus, all cells expressing the cancer-specific biomarker will be differentiated from the non-cancer cells and can aid in the treatment of the cancer (e.g., surgical removal of the cancer or targeted chemotherapy). In another example, cells detectably labeled by a molecular circuit described herein can be isolated away from non-labeled cells. For example, certain types or populations of cells (e.g., B cell populations, T cell populations, or stem cell populations) can be detected and isolated from non-detectably labeled cells. Detectably labeled cells can also be visualized in vivo to determine, e.g., their localization.

The molecular circuits herein can be used to monitor the pharmacokinetics of a compound (e.g., a small molecule compound or a therapeutic protein). For example, a molecular circuit capable of detecting the presence (input cue) of a compound (or group of compounds based on a common molecular feature) can be introduced into a cell and contacted with a compound of interest. In the presence of the input cue (or in some cases in the absence of the input cue), the circuit can produce a detectable output. Such circuits could be useful for determining (i) the permeability of a compound (e.g., permeability of a compound through a cell membrane) or (ii) the stability (half-life or clearance) of a compound in a cell. The cell can also be introduced into an animal model (e.g., a rodent model, a canine model, or a non-human primate model), e.g., to test for the half-life of clearance of a compound from the blood of the animal. The compound can be a small molecule or a therapeutic protein (e.g., an antibody, a growth factor, chemokine, or cytokine).

The circuit can be coupled to an output that provides an indicator for a user. In some embodiments, the circuit is coupled to a mechanism for releasing a therapeutic protein, e.g., an expression or secretion pathway required to produce the therapeutic protein.

In some implementations, a molecular circuit is used to probe individual cells and “label” them without activating a therapy. The therapy can then, optionally, be applied in a separate step that will target the labeled cells. Alternatively, the labeled cells or tissues may be imaged in order to understand the localization of the “positive” cells; e.g., to guide surgery or radiation therapy.

miRNA and transcription factor patterns can, e.g., be used to characterize phenotypes that are of interest for basic research and medicine, as different cell types in a multicellular organism have distinct expression signatures of both miRNA molecules and transcription factors. The same is true for cancer cells and cells obtained from individuals with certain genetic disorders. In some embodiments, an in vivo cell or tissue detection system can be implemented in a subject, e.g., by transient delivery, or stable incorporation into the subject's genome, of the circuits described herein. An application of this method can contain, e.g., the following steps: 1) an operator identifies a tissue or cell type of interest and provides a molecular signature, either as an miRNA and/or transcription factor-based pattern, which is an indicator for this cell type. Alternatively, an operator can use a loose phenotypic definition to identify a list of reliable markers; 2) a circuit is constructed that detects this particular pattern; 3) the components of the circuit are stably incorporated in the germ line of a laboratory animal (e.g. mouse/zebra fish/C. elegans); 4) a transgenic animal (e.g., a fish, a bird, or a non-human mammal) is developed, which “turns on” cells of the desired phenotype in real time. Transgenic laboratory animals can be used for a variety of research purposes. Such transgenic animals can be used, e.g., in studies that follow the development of a certain cell type in real time. The animals can also be used to study positive and negative effects of experimental drug treatments. For example, the fate of certain nervous system cell types can be studied in an Alzheimer disease animal model. The fate of these cell types can be modulated by a variety of experimental drug treatments, and therefore provide a useful research tool for pre-clinical studies.

Multi-color detection systems can be used to, e.g., study multiple (e.g., two, three, four, five, six, seven, eight, nine, or ten or more) tissue types in vivo in a single organism or multiple cell types (or cells at different stages of differentiation) in a mixed population of cells.

Therapeutic Applications. An in vivo operational molecular circuit described herein can be used as a direct therapeutic modality (or combination diagnostic/therapy) for a variety of disorders (e.g., cancer, metabolic disorders, immunological disorders, or infections such as viral, bacterial, or parasitic infections). For example, the components of a molecular circuit can be delivered to a cancer cell, wherein the circuit comprises one or more sensors (mediators) capable of detecting, and responding to, input cues such as, e.g., the expression of an oncogene (e.g., H-ras, BCR-Abl, Bcl-2, or PCT/RET) and/or the lack of expression of a tumor suppressor protein (e.g., p53, BRCA1, RB, APC, or p19ARF). The circuit can, e.g., trigger apoptosis or growth arrest of the cell in response to one of the following input cues: expression of an oncogene or lack of expression of a tumor suppressor, or in Boolean terms, if [Oncogene] OR NOT [tumor suppressor]. Apoptosis can be the result of the expression of a circuit output such as a death receptor (e.g., FasR or TNFR), death receptor ligand (e.g., FasL or TNF), a caspase (e.g., caspase 3 or caspase 9), cytochrome-c, a BH3-containing proapoptotic protein (e.g., BAX, BAD, BID, or BIM), or apoptosis inducing factor (AIF)). Growth arrest can be the result of a circuit output such as p21, p19ARF, p53, or RB protein.

In some embodiments, the circuit can respond to an input cue(s) and trigger the release of a therapeutic agent as the output. (The therapeutic agent can be a protein, an siRNA, an shRNA, a miRNA, a small molecule, or any of the outputs described herein). For example, the molecular circuit introduced into a cell or group of cells suspected of lacking an enzyme, wherein the circuit responds to the input cue of NOT(enzyme) and triggers the production of the enzyme as an output. In one embodiment, the circuit can respond to the lack of glucocerebrosidase in a cell (e.g., a liver cell, a bone marrow cell, or a splenic cell), and trigger the production of a glucocerebrosidase output. Such a molecular circuit could be useful for treating the metabolic disorder Gaucher disease. In some embodiments, the circuit can trigger the production of one or more siRNA (or shRNA) therapeutic agents. For example, where the cell expresses an aberrant form (e.g., an oncogenic form) of a protein (which protein could serve as an input cue), the circuit can trigger the production of one or more siRNAs specific for the mRNA encoding the aberrant protein, thereby ablating its translation. In another example, where a cell is infected with a virus and produces a viral RNA molecule, the circuit (introduced into the cell) can trigger the production of an RNA molecule, such as an siRNA (or shRNA), that interferes with viral viability or propagation within the host cell.

siRNA therapeutic agents could also be used to silence the expression of genes controlling cell cycle control, cell viability, or differentiation, e.g., in cancer cells or microbially-infected cells.

The circuit can be used for local or systemic delivery of one or more therapeutic agents. For example, the circuit can be introduced (transfected) into cells (e.g., without discriminating as to whether a cell if diseased or not), but only release a therapeutic output in or around the diseased cells (e.g., cancer cells or microbially-infected cells). Systemic delivery of one or more therapeutic agents by a molecular circuit can involve, e.g., introducing the circuit into cells, e.g., healthy and/or diseased cells, wherein production and systemic release of one or more therapeutic agents by the circuit is triggered by, e.g.: (a) a disease state cue in diseased cells containing the circuit or (b) the detection of a disease state cue by healthy cells containing the circuit.

A cell comprising a molecular circuit described herein can also be used as a therapeutic. For example, a cell containing a molecular circuit can be placed into a site of an infection, e.g., a localized viral infection, wherein the circuit responds to the presence of a viral nucleic acid and produces interferon (IFN) as an output. In some embodiments, the circuit responds to the presence of Herpes viral RNA as an input cue, such that when the cell containing the circuit is infected with Herpes virus and the virus expresses an RNA (HerpesRNA (TRUE)), the circuit triggers high level production of IFN. In another example, a cell's circuit can respond to one or more pro-inflammatory input cues. For example, a cell containing such a circuit can be introduced into a anatomical site having, suspected of having, or at risk of developing, a pro-inflammatory response (e.g., a joint affected by rheumatoid arthritis). Thus, if the circuit senses, e.g., the presence of a pro-inflammatory cytokine (e.g., IL-1 or TNF) AND elevated temperature, the circuit can trigger the production of an anti-inflammatory cytokine output (e.g., IL-4, IL-6, IL-10, IL-11, or IL-13). In Boolean terms, the circuit would produce an anti-inflammatory cytokine if [high pro-inflammatory cytokine] AND [high temperature].

The molecular circuits described herein can also be used therapeutically to promote, e.g., tissue regeneration, localized production of a secreted protein (e.g., as above), and certain types of immune-like responses.

Protein Expression/Purification Applications. The molecular circuits described herein, and the cells containing the molecular circuits, can be used in a variety of industrial applications including the production (e.g., expression and/or purification) of protein products. For example, molecular circuits can be used to regulate synthesis of products, e.g., in a bioreactor. Such products include protein biologics, protein reagents, and organic molecules, e.g., polyketides. The synthesis of a protein product can be optimized based on a variety of inputs, e.g., nutrient availability, cell density, pH, oxidative stress, and so forth. Circuits can be coupled to sensors for such inputs, either by the introduction of nucleic acid aptamers sensitive to appropriate signals, by coupling to endogenous proteins that detect appropriate signals, or by the introduction of exogenous sensors, e.g., two component systems from bacteria.

The circuits can be implemented such that the circuit output is the transcription of an mRNA for the protein product or a component required for production of the protein product. For example, the mRNA could encode T7 RNA polymerase which could then be used to drive synthesis of the protein product. Accordingly, the circuit can be used to trigger synthesis of a protein product only when desired parameters are achieved, e.g., hi cell density FALSE, low cell density FALSE, oxidative stress FALSE, and nutrient availability TRUE or in Boolean terms:
[nutrient availability] AND NOT ([hi cell density] OR [low cell density] OR [oxidative stress]).

Cell density can be monitored, for example, by further engineering the cells to secrete a detectable protein, e.g., a protein detectable by a cell surface receptor. If the detectable protein has a defined turnover rate, e.g., due to protein degradation, its concentration in a medium can be used as a measure of cell density.

The circuits can implemented in any appropriate host cell, e.g., a bacterial cell (e.g., a Bacillus, a Streptomyces, or an E. coli cell), a fungal cell (e.g., Pichia, Sacchromyces, etc.), a plant cell, or an animal cell (e.g., a mammalian cell suitable for growth in culture, e.g., a CHO cell). Examples of protein products include cytokines (e.g., interferons, interleukins, growth hormone, insulin, etc), recombinant antibodies, protease inhibitors, and other therapeutic proteins. Other examples of protein products include laboratory and purification reagents, e.g., restriction enzymes, protein A, proteases, and so forth.

Other applications include combined diagnostics and therapy. An exemplary circuit will autonomously probe individual cells, determine if the conditions for activating the therapeutic agent hold, and subsequently activate the agent.

Cell lines can be engineered with sophisticated properties. For example, it is possible to engineered membrane-bound protein “mediators”/receptors that will transduce the signals from extracellular space to the intracellular circuit. Circuits can integrate such signals with the detection of freely diffusible small molecules. The engineered cells can respond to regulate tissue regeneration, localized production of a secreted protein, certain types of immune-like responses as a function of these input cues.

It is also possible to engineer cell networks, e.g., a “mix” of recombinant cells that contain different circuits. These cells can generate secreted outputs and respond to inputs from the extracellular environment. In this way they may establish communication among themselves. These cell networks can provide a high degree of complexity to cell therapy, diagnostics and synthetic applications.

### Kits

One or more circuit components described herein can be provided as a kit, e.g., a package that includes one or more containers. In one example, each component, or genetic material encoding it, can be provided in a different container. In another example, two or more components are combined in a container. Such kits are useful for any of the diagnostic, therapeutic, or protein production modalities described herein.

For example, circuit components can be provided as a functional part of a kit to identify individual cells with certain complex molecular signature/phenotype.

In Silico Software

Methods for designing a molecular circuit described herein can vary based on the size and scope of the eventual application (e.g., the number and/or type of mediators, input cues, and outputs). Some design and/or implementation of the molecular circuits described herein can include the use of a computer program that operates in silico. For example, the computer can accept as input information about a Boolean operation and output sequences for actuator components, e.g., nucleic acids encoding siRNA, peptide, protein, gene, or mRNA components. For example, the computer can select response elements for actuators that are unique, e.g., a not found in the genome of the desired host cell (e.g., human, murine, primate, bovine, equine, and so forth). According to the principles described herein, the program can design mRNA's encoding output proteins and that include the actuator elements. The program can also design oligonucleotides and other synthetic components useful for assembling DNA or other genetic components for the system.

In silico methods can be implemented in digital electronic circuitry, or in computer hardware, firmware, software, or in combinations thereof. Software can be implemented in a computer program product tangibly embodied in a machine-readable storage device for execution by a programmable processor; and method actions can be performed by a programmable processor executing a program of instructions to perform functions of the invention by operating on input data and generating output.

The following are non-limiting examples.

### EXAMPLES

### Example 1

### Materials and Methods

siRNA molecules. The following ribo-oligonucleotides were used:

T1:

(SEQ ID NO: 1)

CGUACGCGGAAUACUUCGAAUU (sense)
and

(SEQ ID NO: 2)

UUCGAAGUAUUCCGCGUACGUG (antisense);
T2:

(SEQ ID NO: 3)

CGUUGCUAGUACCAACCCUAUU (sense)
and

(SEQ ID NO: 4)

UAGGGUUGGUACUAGCAACGCU (antisense);
SI4:

(SEQ ID NO: 5)

GGCAAGCUGACCCUGAAGUUUG (sense)
and

(SEQ ID NO: 6)

AACUUCAGGGUCAGCUUGCCGU (antisense);
FF3:

(SEQ ID NO: 7)

CGAUAUGGGCUGAAUACAAAUU (sense)
and

(SEQ ID NO: 8)

UUUGUAUUCAGCCCAUAUCGUU (antisense);
FF4:

(SEQ ID NO: 9)

GCUUGAAGUCUUUAAUUAAAUU (sense)
and

(SEQ ID NO: 10)

UUUAAUUAAAGACUUCAAGCGG (antisense).

T1, T2 and SI4 oligomers were obtained from Sigma-Proligo (Sigma Aldrich, St. Louis, Mo.). T1 and T2 were ordered as separate gel-purified oligomers and SI4 as a gel-purified and annealed duplex. FF3 and FF4 oligomers were obtained desalted and deprotected from Dharmacon (Chicago, Ill.), gel-purified in house using 20% denaturing PAGE and standard purification techniques and were subsequently annealed. To anneal RNA oligomers, equimolar amounts of the sense and antisense oligomers (50 or 200 pmol/μL as judged either from the manufacturer's data or NanoDrop measurements) were mixed in 10 to 20 μL of 50 mM NaCl, 10 mM Tris-HCl pH 8.0 and 1 mM EDTA and 0.5 u/μL of Superase-In RNAse inhibitor (Ambion), heated to 95° C. and slowly cooled down to 10° C. in a PCR machine block for 50 min. The size and purity of the annealed products were subsequently verified using 3.5% Metaphor agarose gel (3.5%) and ethidium bromide staining.

Recombinant DNA constructs. Single-target derivatives and multiple-target clause molecules employed in DNF evaluators were derivatives of the pZsYellow-C1 vector (Clontech, Mountain View, Calif.). The vector was restricted by BamHI and XhoI and the digested molecule was purified from agarose gel using a gel purification kit (Qiagene). The DNA inserts into 3′-UTR with ready-to-ligate sticky ends were obtained from Sigma-Proligo, either gel-purified and phosphorylated (T1, T2 and SI4 target oligomers) or desalted (FF3, FF4, FF3x3 and FF4x3 target oligomers). A stop codon was inserted 10 by ahead of the siRNA target sites. The sequences were:

T1 target:

(SEQ ID NO: 11)

TCGAGCTTAACAAGCTTCGACACGTACGCGGAATACTTCGAAG
(sense)
and

(SEQ ID NO: 12)

GATCCTTCGAAGTATTCCGCGTACGTGTCGAAGCTTGTTAAGC
(antisense);
T2 target:

(SEQ ID NO: 13)

TCGAGCTTAACAAGCTTCGAAGCGTTGCTAGTACCAACCCTAG
(sense)
and

(SEQ ID NO: 14)

GATCCTAGGGTTGGTACTAGCAACGCTTCGAAGCTTGTTAAGC
(antisense);
SI4 target:

(SEQ ID NO: 15)

TCGAGCTTAACAAGCTTCGAACGGCAAGCTGACCCTGAAGTTG
(sense)
and

(SEQ ID NO: 16)

GATCCAACTTCAGGGTCAGCTTGCCGTTCGAAGCTTGTTAAGC
(antisense);
FF3 target:

(SEQ ID NO: 17)

TCGAGCTTAACAAGCTTCGAAACGATATGGGCTGAATACAAAG
(sense)
and

(SEQ ID NO: 18)

GATCCTTTGTATTCAGCCCATATCGTTTCGAAGCTTGTTAAGC
(antisense);
FF4 target:

(SEQ ID NO: 19)

TCGAGCTTAACAAGCTTCGACCGCTTGAAGTCTTTAATTAAAG
(sense)
and

(SEQ ID NO: 20)

GATCCTTTAATTAAAGACTTCAAGCGGTCGAAGCTTGTTAAGC
(antisense);
FF3 × 3 (triple tandem repeat) target:

(SEQ ID NO: 21)

TCGAGCTTAACAAGCTTCGAAACGATATGGGCTGAATACAAAAACGATAT
GGGCTGAATACAAAAACGATATGGGCTGAATACAAAG (sense)
and

(SEQ ID NO: 22)

GATCCTTTGTATTCAGCCCATATCGTTTTTGTATTCAGCCCATATCGTTT
TTGTATTCAGCCCATATCGTTTCGAAGCTTGTTAAGC (antisense);
FF4 × 3 (triple tandem repeat) target:

(SEQ ID NO: 23)

TCGAGCTTAACAAGCTTCGACCGCTTGAAGTCTTTAATTAAACCGCTTGA
AGTCTTTAATTAAACCGCTTGAAGTCTTTAATTAAAG (sense)
and

(SEQ ID NO: 24)

GATCCTTTAATTAAAGACTTCAAGCGGTTTAATTAAAGACTTCAAGCGGT
TTAATTAAAGACTTCAAGCGGTCGAAGCTTGTTAAGC (antisense).

Multiple-target constructs were constructed in a similar fashion. All oligonucleotides were obtained desalted from Sigma-Proligo, gel-purified in house using 8% denaturing PAGE, annealed, and the double-stranded inserts were phosphorylated prior to ligation. The oligomers were:

T1.T2.SI4 construct:

(SEQ ID NO: 25)

TCGAGCTTAACAAGCTTCGACACGTACGCGGAATACTTCGAAAGCGTTGC
TAGTACCAACCCTAACGGCAAGCTGACCCTGAAGTTG (sense)
and

(SEQ ID NO: 26)

GATCCAACTTCAGGGTCAGCTTGCCGTTAGGGTTGGTACTAGCAACGCTT
TCGAAGTATTCCGCGTACGTGTCGAAGCTTGTTAAGC (antisense);
FF3.FF4 construct:

(SEQ ID NO: 27)

TCGAGCTTAACAAGCTTCGAAACGATATGGGCTGAATACAAACCGCTTGA
AGTCTTTAATTAAAG (sense)
and

(SEQ ID NO: 28)

GATCCTTTAATTAAAGACTTCAAGCGGTTTGTATTCAGCCCATATCGTTT
CGAAGCTTGTTAAGC (antisense);
FF4.T1.SI4 construct:

(SEQ ID NO: 29)

TCGAGCTTAACAAGCTTCGACCGCTTGAAGTCTTTAATTAAACACGTACG
CGGAATACTTCGAAACGGCAAGCTGACCCTGAAGTTG (sense)
and

(SEQ ID NO: 30)

GATCCAACTTCAGGGTCAGCTTGCCGTTTCGAAGTATTCCGCGTACGTGT
TTAATTAAAGACTTCAAGCGGTCGAAGCTTGTTAAGC (antisense);
FF3.T2 construct:

(SEQ ID NO: 31)

TCGAGCTTAACAAGCTTCGAAACGATATGGGCTGAATACAAAAGCGTTGC
TAGTACCAACCCTAG (sense)
and

(SEQ ID NO: 32)

GATCCTAGGGTTGGTACTAGCAACGCTTTTGTATTCAGCCCATATCGTTT
CGAAGCTTGTTAAGC (antisense).

The double stranded inserts were obtained by annealing 250 pmol of the sense and antisense oligomers in 10 to 20 μL of TE buffer supplemented with 50 mM NaCl, in a PCR machine block by heating to 95° C. and cooling down to 10° C. for 50 min. Duplex formation was confirmed by 3.5% Metamorph agarose (Cambrex). 150 pmol of each insert were 5′-phosphorylated in 50 μL of PNK buffer (New England Biolabs) by 15 units of PNK (New England Biolabs) and 1 mM ATP (Invitrogen) for 30 min at 37° C.

The inserts were ligated into a digested pZsYellow vector by using ˜2:1 molar ratio of the insert to the vector and 25 ng of the vector in 10 μL of T4 DNA Ligase buffer (New England Biolabs) using 400 units of T4 DNA Ligase (New England Biolabs) for 2.5 hours at 15° C. The reaction mixture was transformed into 50 μL of Max Efficiency DH5α E. coli cells (Invitrogen, Cat # 18258-012), outgrown for 60 min at 37° C. in SOC medium (Invitrogen) shaken at 300 rpm and plated on LB-Kan plates overnight. The colonies were analysed by colony PCR using a pair of primers flanking the insert region. Positive colonies were expanded in LB-Kan medium overnight, plasmid DNA was isolated using MiniPrep kit (Qiagen) and the insert integrity was verified by sequencing (Genewiz).

The constructs used to evaluate a CNF expression were constructed as follows. LacI-derived clause molecule: LacI gene was amplified from the pCMVLacI plasmid from the LacSwitch II Inducible Mammalian Expression System kit (Stratagene) using the primers

| | |
|---|---|
| CCAGCTAGCGAGGTACCCTCCCACCATG | (SEQ ID NO: 33) |
| and | |
| CCAAGATCTTCAAACCTTCCTCTTCTTCTTAGG | (SEQ ID NO: 34) |

with engineered BglII and NheI restriction sites. The PCR product was purified using PCR purification kit (Qiagen, Valencia, Calif.), digested by BglII and NheI enzymes and purified again from the short digested fragments using the same kit. In parallel, the ZsYellow gene was excised from the ZsYellow-derived clause molecules by digestion with BglII and NheI enzymes and the backbone vector lacking the ZsYellow gene was gel-purified from 1% agarose using Gel Purification kit (Qiagen). The digested LacI insert was ligated into the digested vector at a 1:2 molar ratio as described above, transformed into the Max Efficiency DH5 E. coli cells and plated on LB-Kan plates overnight. The colonies were verified by colony PCR using the following primers:

| | |
|---|---|
| CGTCAATGGGAGTTTGTTTTG | (SEQ ID NO: 35) |
| and | |
| GCGCCGAGACAGAACTTAAT, | (SEQ ID NO: 36) |

and further by sequencing the LacI and the 3′-UTR insert regions.

To construct CMV-LacI-F3x3 and CMV-LacI-F4x3, the CMV-LacI-T2 plasmid made as described above was digested by BamHI and XhoI enzymes to excise the T2 target site, and the backbone vector lacking the target site was gel purified from 1% agarose using a Gel Purification kit (Qiagen). The annealed oligos F3x3 and F4x3 were phosphorylated and ligated into the digested vector at a 1:2 molar ratio, transformed into Max Efficiency DH5α E. coli cells and plated on LB-KAN plates overnight. The constructs were verified by sequencing. Construction of the reporter plasmid pCAGOP-DsRed-Monomer-N1 was done as follows: The CMV promoter in pDsRed-Monomer-N1 (Clontech, Mountain View, Calif.) was replaced by a human EF1-α promoter flanked by PacI and EcoRI sites to construct pHef1α-DsRed-Monomer-N1. The plasmid pCAGOP containing the synthetic chicken β-actin promoter with lac opertators (CAGOP) was obtained from Dr. Binétruy (Caron et al. (2005) Cell. Mol. Life Sci. 62 1605-1612). The CAGOP promoter was PCR-amplified using primers

(SEQ ID NO: 37)

5′-ACTAGGTTAATTAATAGT TATTAATAGTAATCAATTACGG-3′
and

(SEQ ID NO: 38)

5′-GATGAAGAATTCAGGCCGAGGCGGCCGTCGACGTTAACGCTAGCGGC
CGTAATGGCCTACCTGTGGGAGTAACGCGGTCAG-3′.

The PCR product and pHef1α-DsRed-Monomer-N1 were digested with PacI and EcoRI and ligated to construct pCAGOP-DsRed-monomer-N1.

CMV-LacI-KRAB-F3x3-F4x4: pCMV-LacI-KRAB was constructed from plasmid pCMV-LacI (Stratagene) where LacI is driven by a CMV promoter and pLV-tTRKRAB-Red (a gift of Prof. Didier Trono, EPFL Switzerland) which contains the KRAB repression domain. A PCR of pCMV-LacI was done using primers

(SEQ ID NO: 39)

5′-ACTAAGCACCTGCACTCCAGGAACGCACGGGTGTTG
GGTCGTTTG-3′
and

(SEQ ID NO: 40)

5′-CTAGATCACCTGCATCACTGCCCGCTTTCCAG
TCGGGAAACCTG-3′

to eliminate the stop codon of the LacI gene and introduce AarI sites. A PCR of pLV-tTRKRAB-Red was done using primers

(SEQ ID NO: 41)

5′-CTAATCCACCTGCACTCGCAGCCAAAAAAGAAGAGAAAGGTCGA
C-3′
and

(SEQ ID NO: 42)

5′-ATCATCCACCTGCATCACCTGTTAAACTGATGATTTGATTTCAAATG
C-3′

to amplify a fragment containing the KRAB domain flanked by AarI sites. Digestion of the PCR fragments using AarI and subsequent ligation resulted in the cloning of pCMV-LacI-KRAB with the KRAB repression domain fused in frame at the C-terminal of LacI. The LacI-KRAB fragment was PCR amplified using primers

(SEQ ID NO: 43)

5′-ACTACTGCTAGCTCCCACCATGAAACCAGTAACG-3′
and

(SEQ ID NO: 44)

5′-CATCATAGATCTTTAAACTGATGATTTGATTTCAAATG-3′.

The PCR product was cloned into pZsYellow-C1-FF3-FF4 using the BglII and NheI sites to create pCMV-LacI-KRAB-FF3-FF4.

The insert F3x3-F4x3 was constructed by ligating two fragments, F34x3.I and F34x3.II. The first fragment was obtained by annealing a non-phosphorylated oligonucleotide F34x3.I.S,

(SEQ ID NO: 45)

TCGAGCTTAACAAGCTTCGAAACGATATGGGCTGAATACAAAAACGATAT
GGGCTGAATACAAAAACGATATG

5′-phosphorylated oligonucleotide F34x3.I.AS,

(SEQ ID NO: 46)

CAGCCCATATCGTTTTTGTATTCAGCCCATATCGTTTTTGTATTCAGCCC
ATATCGTTTCGAAGCTTGTTAAGC.

The second fragment was obtained by annealing a phosphorylated oligonucleotide F34x3.II.S,

(SEQ ID NO: 47)

GGCTGAATACAAACCGCTTGAAGTCTTTAATTAAACCGCTTGAAGTCTTT
AATTAAACCGCTTGAAGTCTTTAATTAAAG

and a non-phosphorylated oligonucleotide F34x3.II.AS,

(SEQ ID NO: 48)

GATCCTTTAATTAAAGACTTCAAGCGGTTTAATTAAAGACTTCAAGCGGT
TTAATTAAAGACTTCAAGCGGTTTGTATT.

The ligated insert was gel-purified, phosphorylated and subloned into pCMV-LacI-KRAB-FF3-FF4 after excision of the FF3-FF4 insert with BamHI and XhoI enzymes to afford CMV-LacI-KRAB-F3x3-F4x4 construct.

Cell culture. 293-H Cells, SFM Adapted (Invitrogen, Cat # 11631-017) were used throughout the experiments. The cells were grown at 37° C., 100% humidity and 5% CO₂. The cells were initially transferred into CD-293 medium and a week later moved to the Dulbecco's modified Eagle's medium (DMEM, Invitrogen, Cat # 11965-11810) supplemented with 0.1 mM of MEM non-essential amino acids (Invitrogen, Cat # 11140-050), 0.045 units/mL of Penicillin and 0.045 μg/mL Streptomycin (Pennicillin-Streptomycin liquid, Invitrogen) and 10% Fetal Bovine Serum (FBS, Invitrogen). The adherent culture was maintained indefinitely in this medium by trypsinizing with Trypsin-EDTA (0.25% Trypsin with EDTAx4Na, Invitrogen) and diluting in a fresh medium upon reaching 50-90% confluence.

For transfection experiments, ˜90-120 thousand cells in 1 mL of complete medium were plated into each well of 12-well uncoated glass-bottom (MatTek) or plastic (Falcon) plates and grown for ˜24 hours. Shortly before transfection, the medium was replaced with 1 mL DMEM without supplements with a single medium wash step. Transfection mixtures were prepared by mixing all nucleic acids, including the plasmids and the siRNAs into 40 μL of DMEM. 2.4 μL of the Plus reagent (Invitrogen) was added to the final mix and incubated for 20 min at room temperature. In parallel, 1.6 μL Lipofectamine (Invitrogen) were mixed with 40 μL DMEM. Plus- and Lipofectamine-containing solutions were mixed and incubated for 20 more minutes at room temperature prior to application to the cells. The transfection mixture (typically 90 μL) was applied to the wells and mixed with the medium by gentle shaking. Three hours after transfection, 120 μL FBS was added to the wells and the cells were incubated for up to 48 hours before the analysis.

The cells were prepared for the FACS analysis by trypsinizing each well with 0.5 mL 0.25% trypsin-EDTA, collecting the cell suspension and centrifuging at 5000 rpm for 2 min. Trypsin was removed and the pellet resuspended by short vortexing in 0.5 mL PBS buffer (Invitrogen).

Microscope measurements and image processing. All microscope images were taken from live cells grown in glass-bottom wells (MatTek) in the transfection medium supplimented with 10% FBS. We used the Zeiss Axiovert 200 microscope equipped with Sutter filter wheels, Prior mechanized stage and an environmental chamber (Solent) held at 37° C. during measurements. The images were collected by Orca ERII camera cooled to −60° C., in the high precision (14 bit) mode using a 20× PlanApochromat NA 0.8, PH2 objective. The collection setting for the fluorophores in crosstalk measurements and DNF evaluation experiments were 500/20x (excitation) and 535/30m (emission) filters for ZsYellow; and 430/25x (excitation) and 470/30m (emission) for AmCyan. A filter cube Sedat Quad (Chroma 86004v2) was used for both fluorophores. In CNF evaluation experiments the settings were: 565/55x (excitation) and 650/70 (emission) filters with a filter cube GFP/mRFP1 (Chroma 86021) for dsRed-monomer, and amCyan settings as above. For the anticorrelated output experiment we used YFP setting as above and 565/25 (excitation) and 650/70 (emission) filters with a filter cube GFP/tdimer (Chroma 86077) for dsRed-monomer. Data collection and processing was performed by the METAMORPH™ 7.0 software (Molecular Devices). Following background subtraction, the relative intensities of the internal transfection control and the reporter protein were adjusted in such a way as so to equalize the apparent intensity of both in the negative control experiments. The settings were applied uniformly to all images taken from the crosstalk experiments and DNF evaluations. A different setting was applied to the images taken from the CNF evaluation experiments and to the anti-correlated output experiments due to the different baseline fluorescence of the constructs.

### Example 2

This example illustrates that for a given Boolean expression in one of the standard forms, the combination of siRNA modulation by the decision cues and the arrangement of the siRNA targets in the downstream gene components allows for the construction of an automaton that evaluates that expression.

A molecular circuit comprising a single cue, its mediator siRNA and the mRNA of the actuator gene modified by this siRNA's target in its 3′-untranslated region (UTR)²² was constructed. In this circuit, inactivation of the siRNA by the cue results in a high actuator level and its activation downregulates the actuator. In Boolean terms, the former case corresponds to evaluating a single-variable expression (cue) that represents a logic variable substituting the physical cue molecule (circuit Ia in FIG. 5A), while the latter evaluates the expression NOT(cue) (circuit IIa in FIG. 5A). When the target modifies the 3′-UTR of the transcription factor that represses the actuator instead of the actuator mRNA itself, activation or inactivation of the siRNA by the cue will, respectively, activate or inactivate the actuator. Similarly, this corresponds to evaluating the expressions (cue) (circuit Ib in FIG. 5B) and NOT(cue) (circuit IIb in FIG. 5B).

The above analysis shows that a negation of a variable, the only type of negation that appears in the standard forms, may be implemented in the sensing step. Therefore, siRNA mediators and their targets can be associated with cue variables or their negations, i.e. literals, depending on how the siRNAs are regulated by the cues and whether the targets are fused into the 3′-UTR of an actuator or its repressor (FIGS. 5A, 5B, and 5C). With this sensing mechanism in place, a circuit that evaluates single literals can be scaled up to evaluate larger expressions by adding siRNA targets and actuator gene variants. A first implementation includes placing a number of different mediator siRNA targets in the 3′-UTR of the actuator. In this arrangement, any siRNA mediator downregulates actuator expression. Therefore, all literals that have their associated targets in the 3′-UTR of the actuator must be True in order for it to be expressed (FIG. 5D), which implements an AND logic operation. An actuator thus modified evaluates an AND clause and is hereinafter referred to as a clause molecule. Moreover, constructs comprising the actuator modified by different sets of targets in its 3′-UTR may be used in parallel. If a TRUE evaluation result is defined as the expression level of an actuator protein obtained from a single clause construct, the parallel arrangement implements an OR operation between the clauses. This definition is common in the field of molecular logic (Kramer et al. (2004) Biotech and Bioeng. 87, 478-484 and Seelig et al. (2006) Science 314, 1585-1588) and it makes biological sense when the amount of actuation obtained from a single clause already saturates the downstream response process. This exemplifies the construction of a DNF evaluator.

When the targets are placed in the 3′-UTR of a repressor, one TRUE literal is sufficient to trigger the expression of the actuator. This property implements an OR operation among literals and the modified repressor is an OR clause molecule. Using the same repressor modified by different sets of targets in parallel implements an AND operation between clauses. In this case the TRUE evaluation result is defined by the actuation level obtained when all clause molecules are downregulated. FALSE evaluation result is, on the other hand, defined as the actuation level obtained with at least one clause molecule fully active. This circuit design combined with the interpretation rules for the evaluator's output complete a construction of the CNF evaluator (FIG. 5E).

The above system can be implemented in mammalian cells or any other cells that have RNAi machinery. LacI was chosen as the repressor since it is foreign to mammalian cells and may be used without interfering with endogenous processes (Caron et al. (2005) Cell. Mol. Life Sci. 62 1605-1612). The feasibility of the evaluation framework was experimentally determined by transfecting cells with the clause constructs and adding, or withholding, siRNA molecules to represent presence or absence of the cues that appear in expression (FIG. 5C). The reporter protein ZsYellow was used to represent the actuator in the DNF expressions and dsRed-monomer in CNF ones. Plasmids expressing amCyan protein served as an internal transfection control.

The structure of the mediator siRNA molecules, as well as their targets, depends on their regulatory cues. Derivatives of known siRNAs and their targets for the current implementation were selected, and constructed five siRNA-target pairs based on published sequences derived from non-mammalian genes (T1 and T2 from renilla (Elbashir et al. (2001) Nature 411, 494-498 and Kobayashi et al. (2004) J. Pharm. Exp. Therap. 308, 688-693) and FF3 and FF4 from firefly luciferases (Reynolds et al. (2004) Nat. Biotechnol. 22, 326-330) and SI4 from eGFP (Sullivan et al. (2005) J. Virol. 79, 7371-7379)) to represent up to five cues. The sequences were modified by sliding them along their parental genes to afford at least a pair of A/U bases on the 5′-end of the molecule and a pair of C/G bases on the 3′-end in order to ensure asymmetry in RISC complex assembly (Schwarz et al. (2003) Cell 115, 199-208).

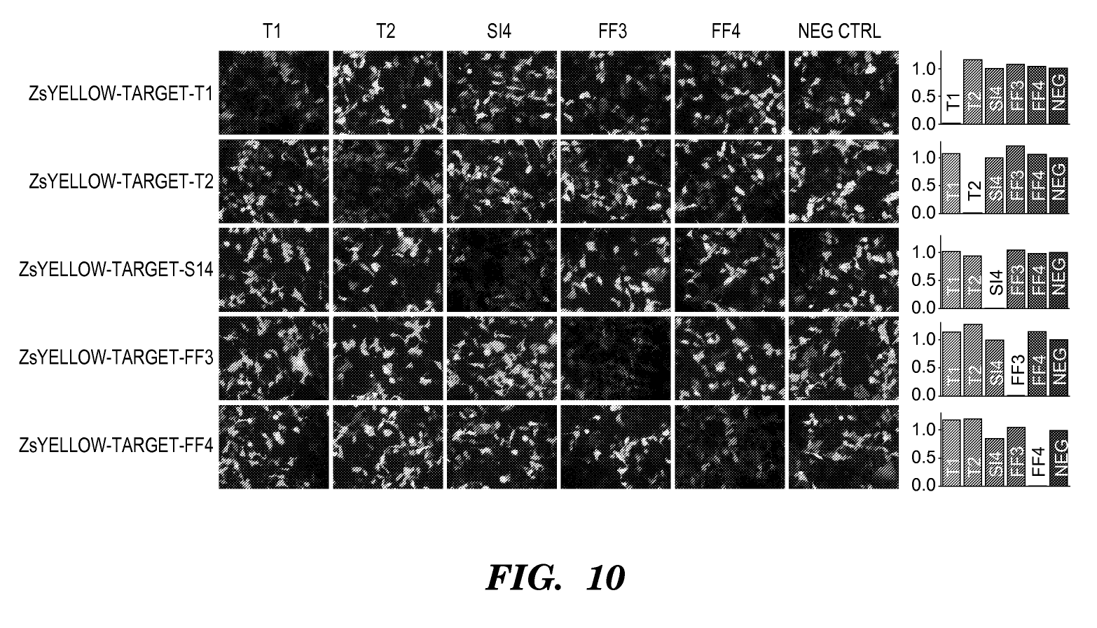

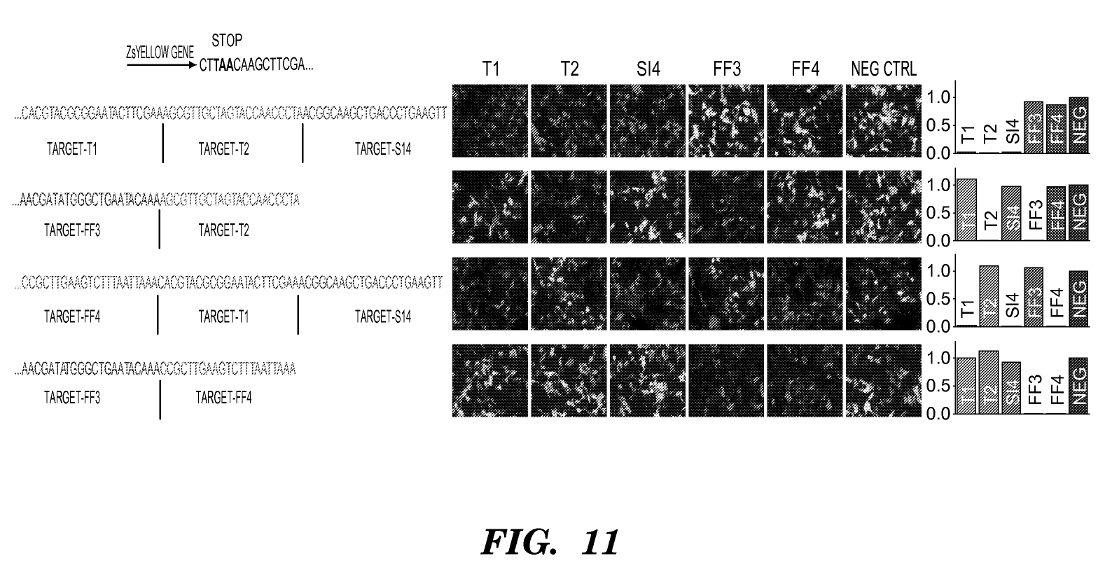

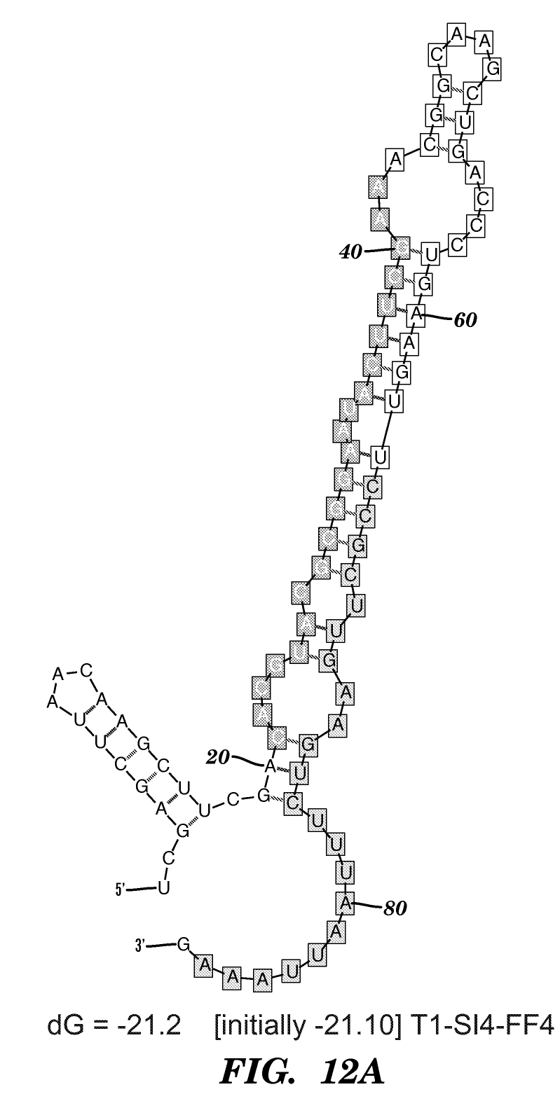

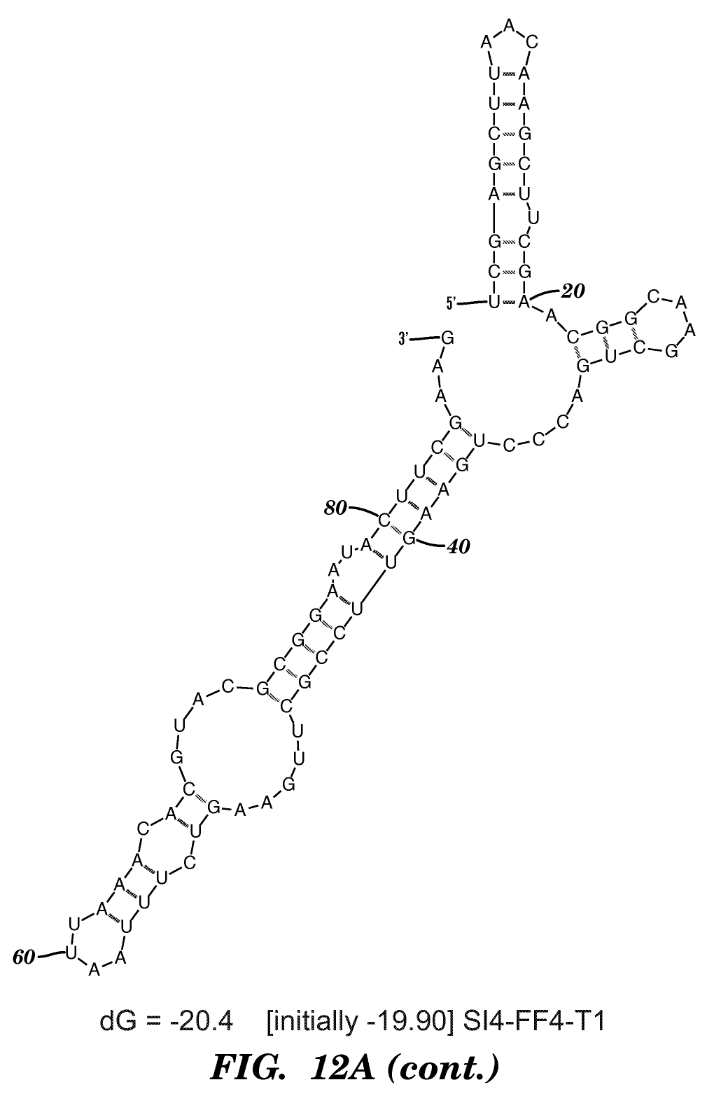

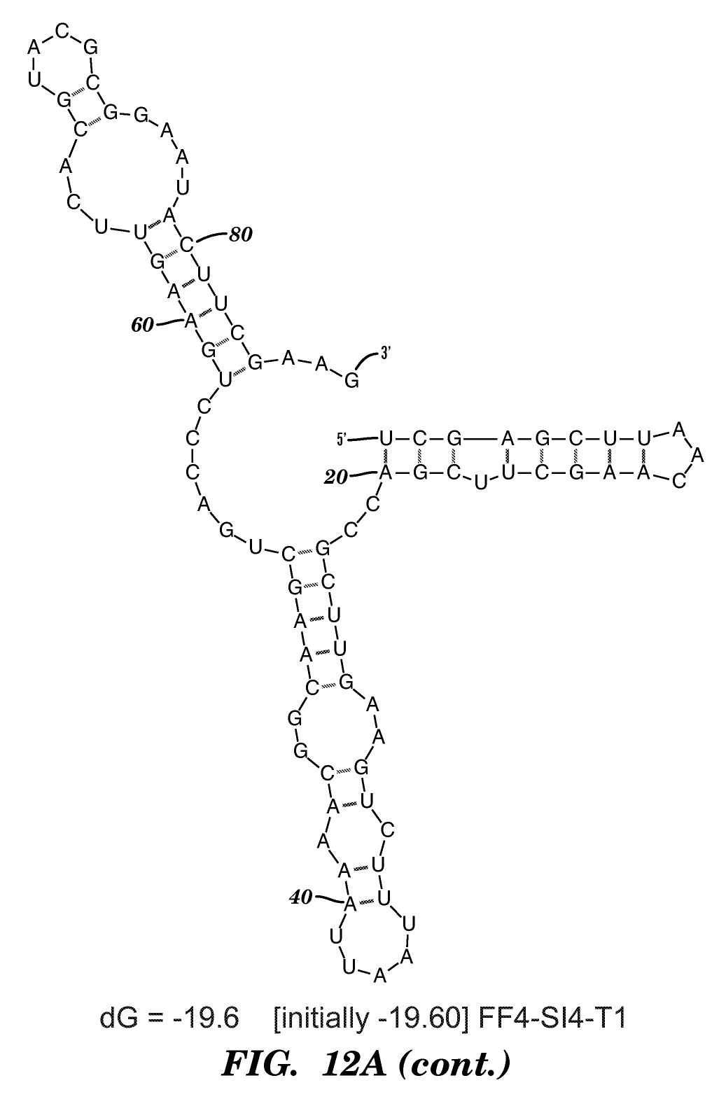

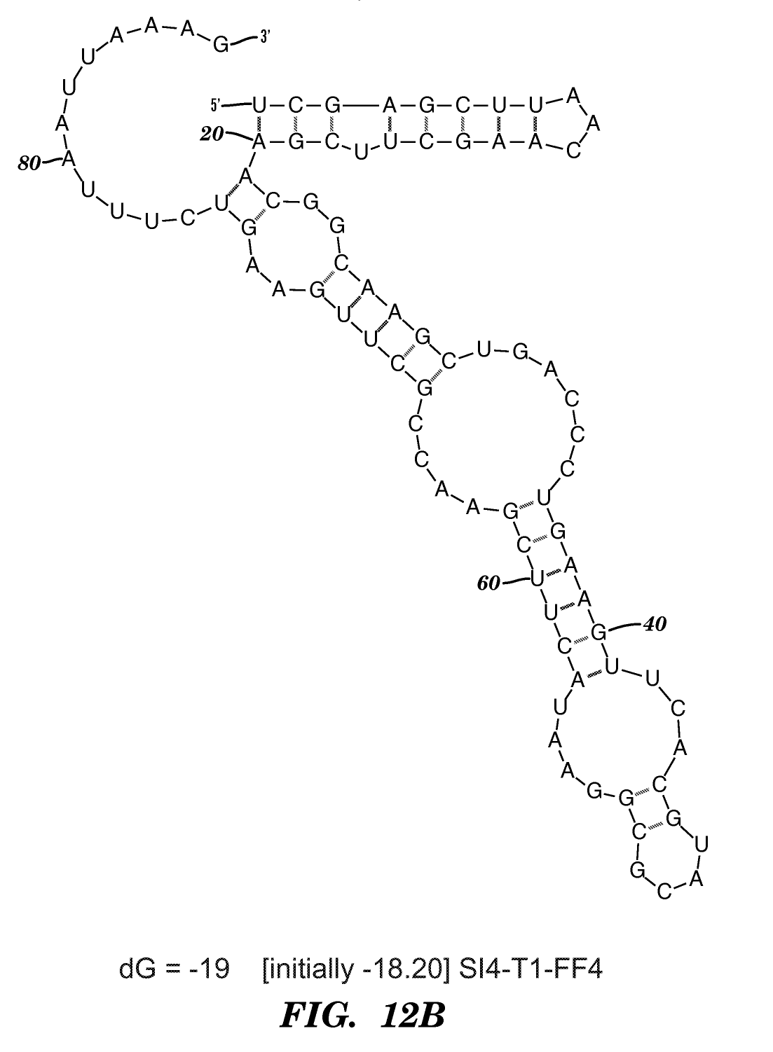

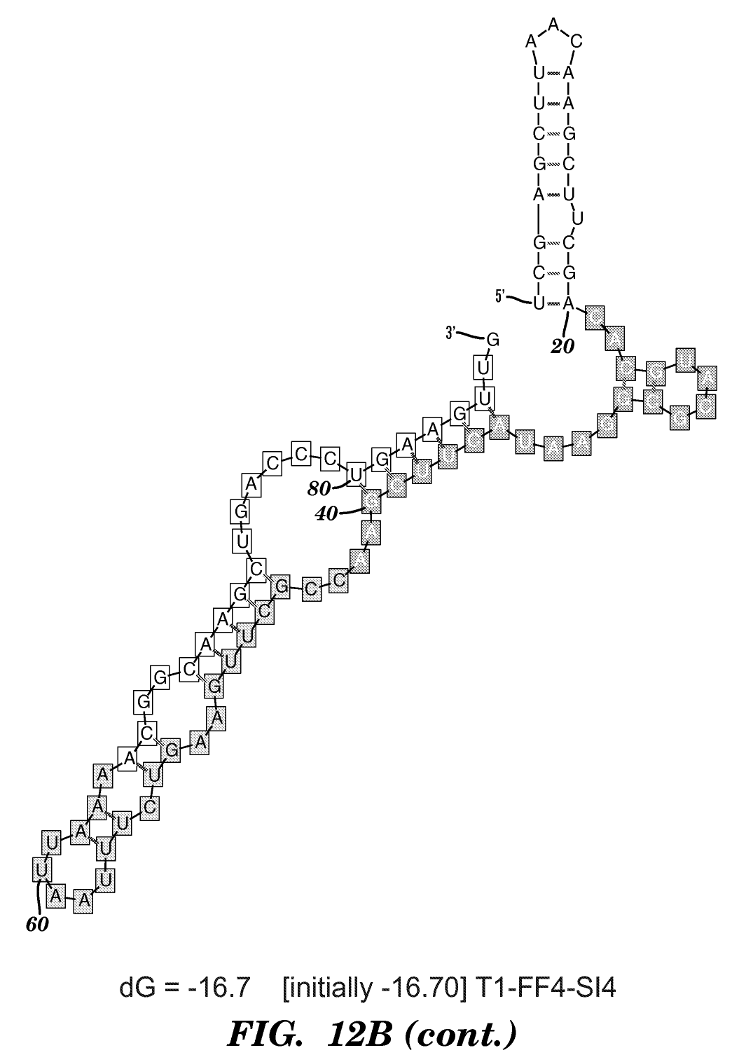

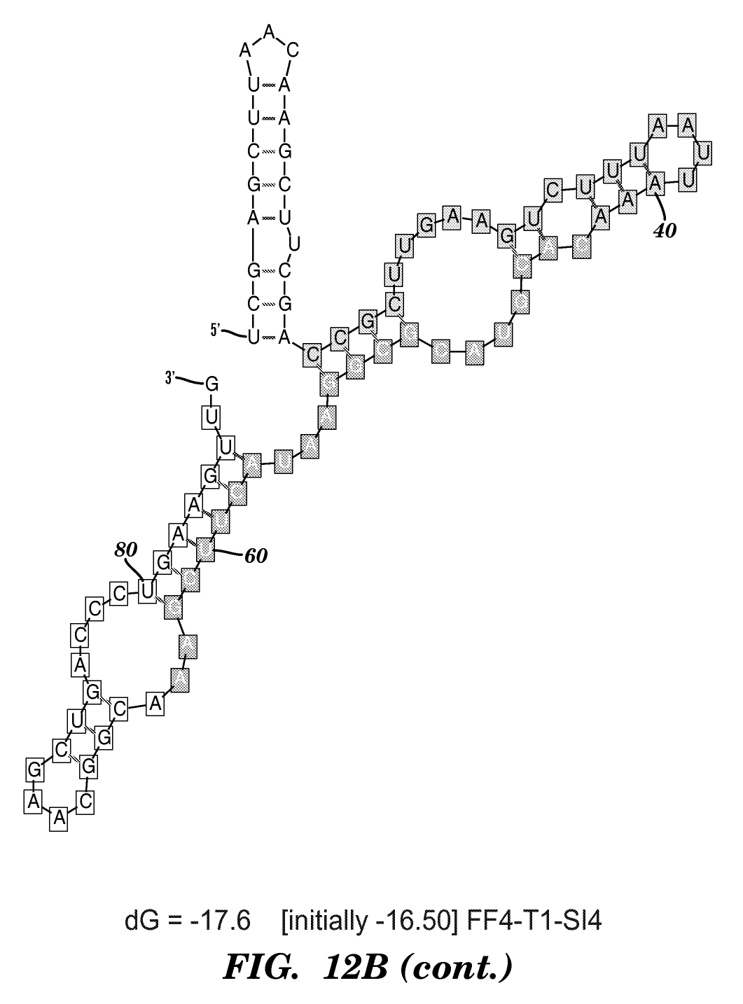

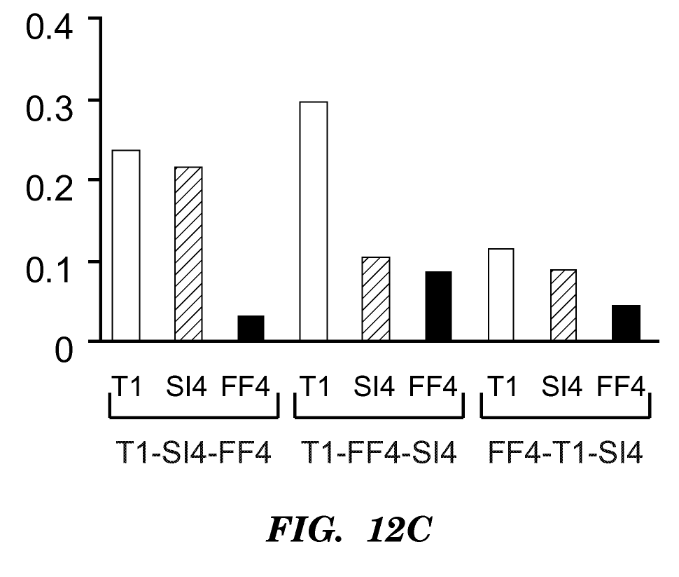

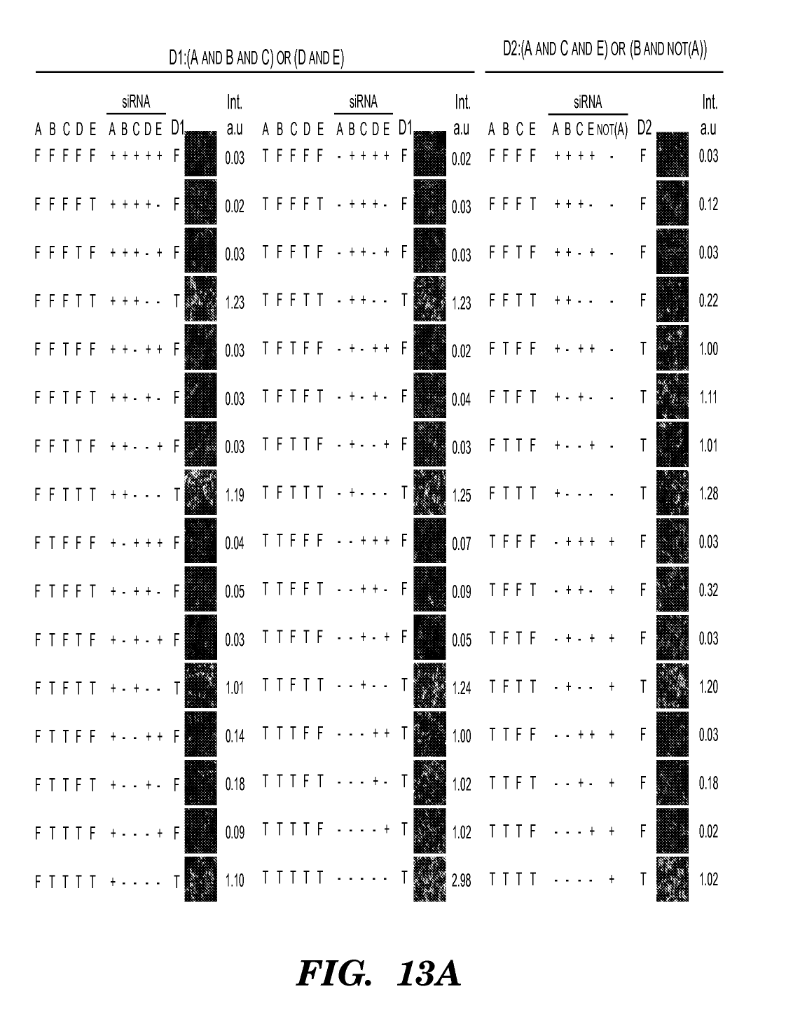

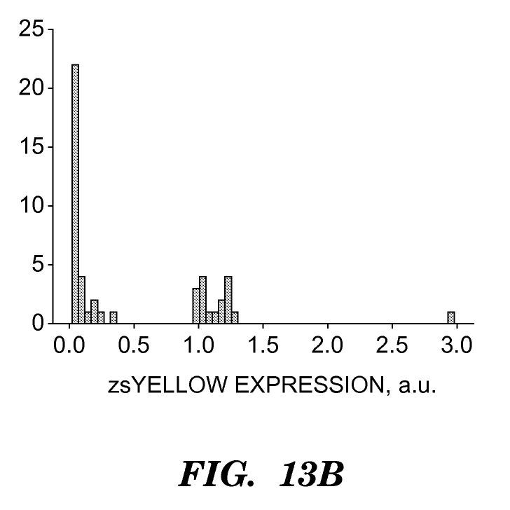

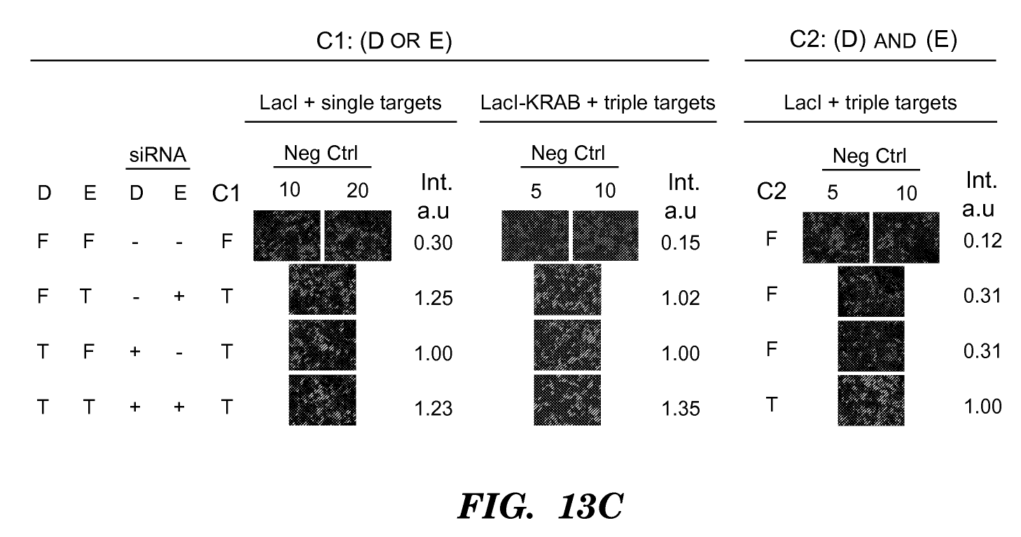

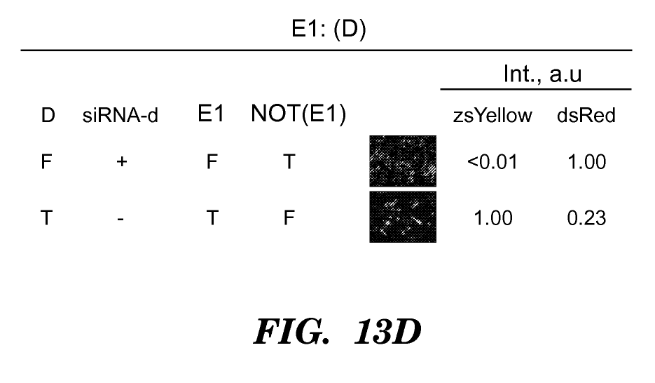

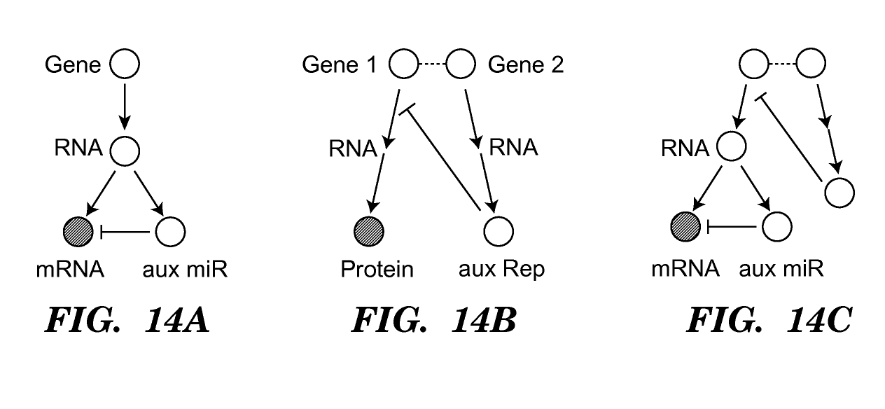

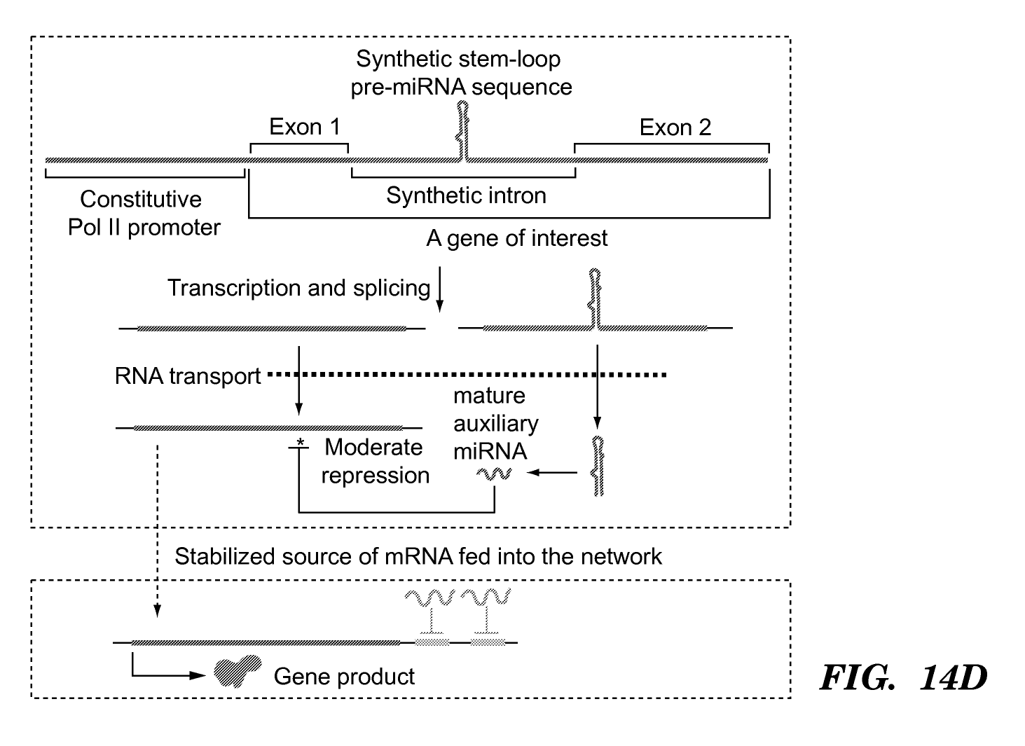

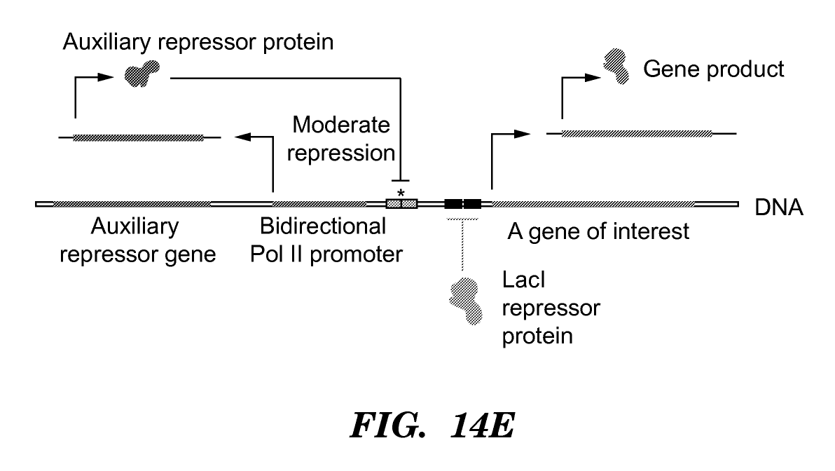

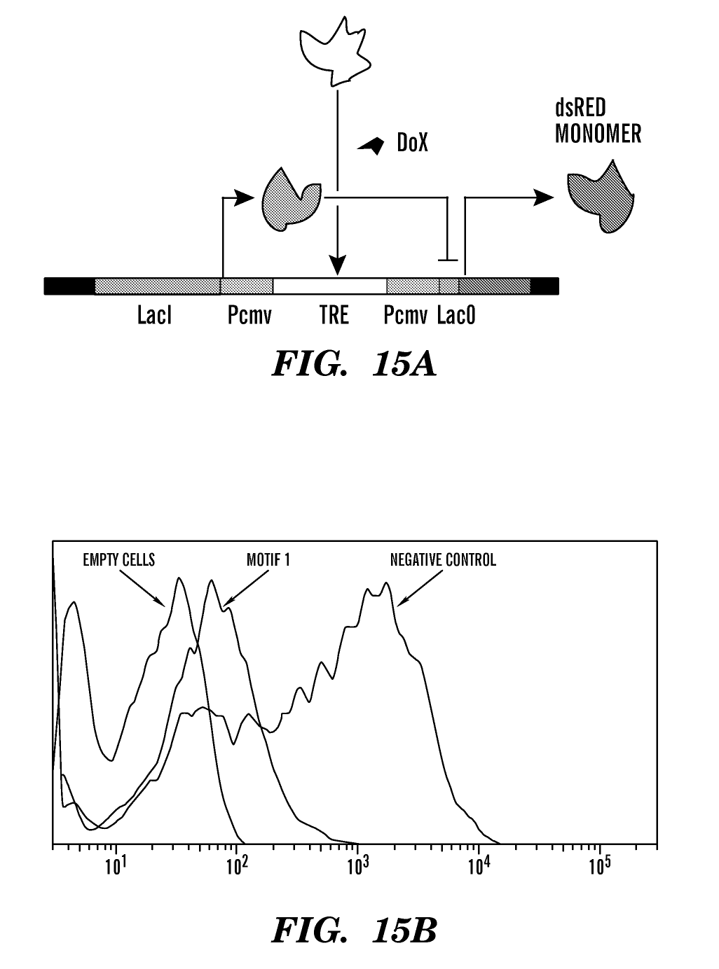

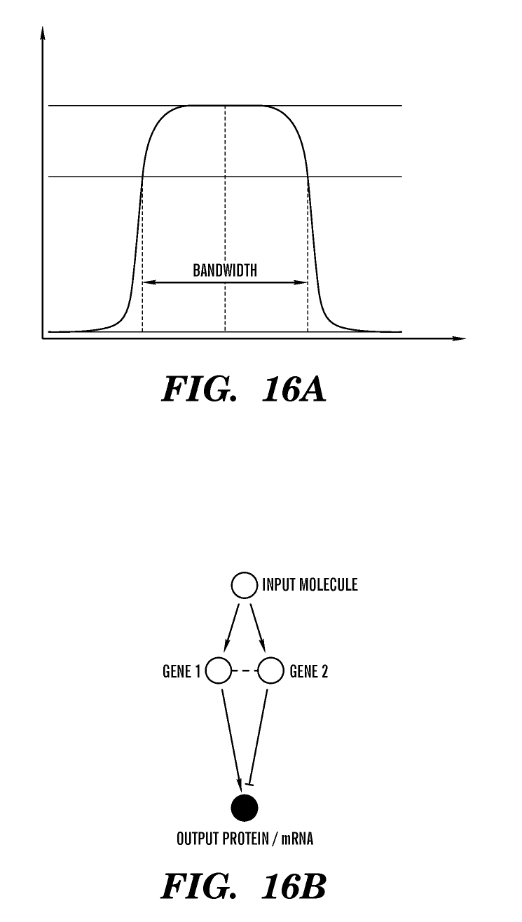

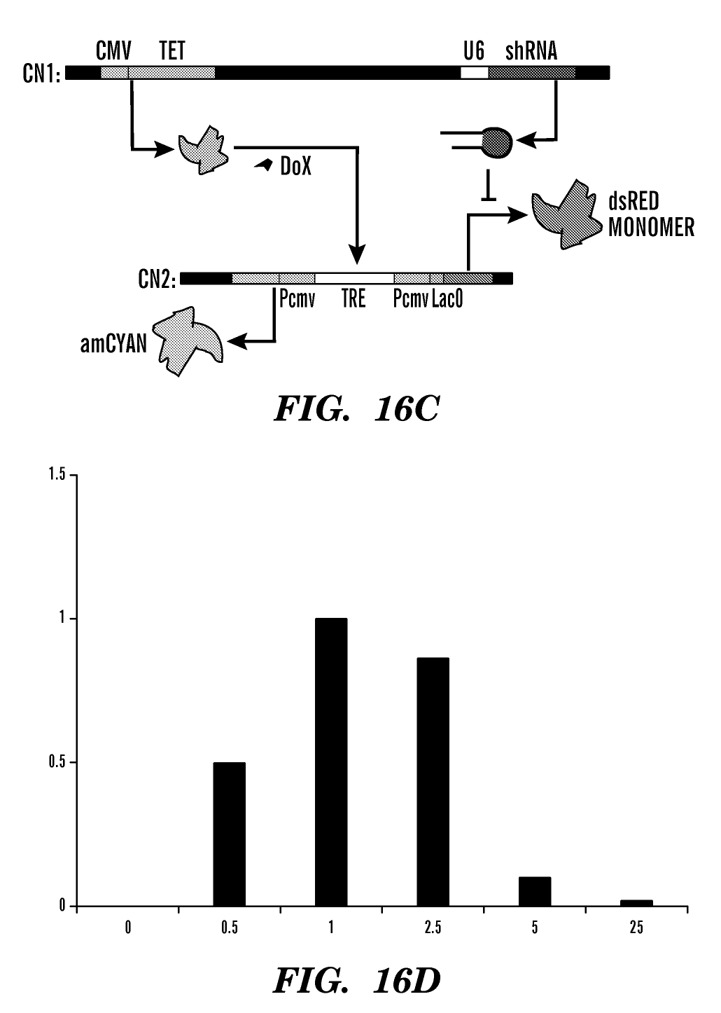

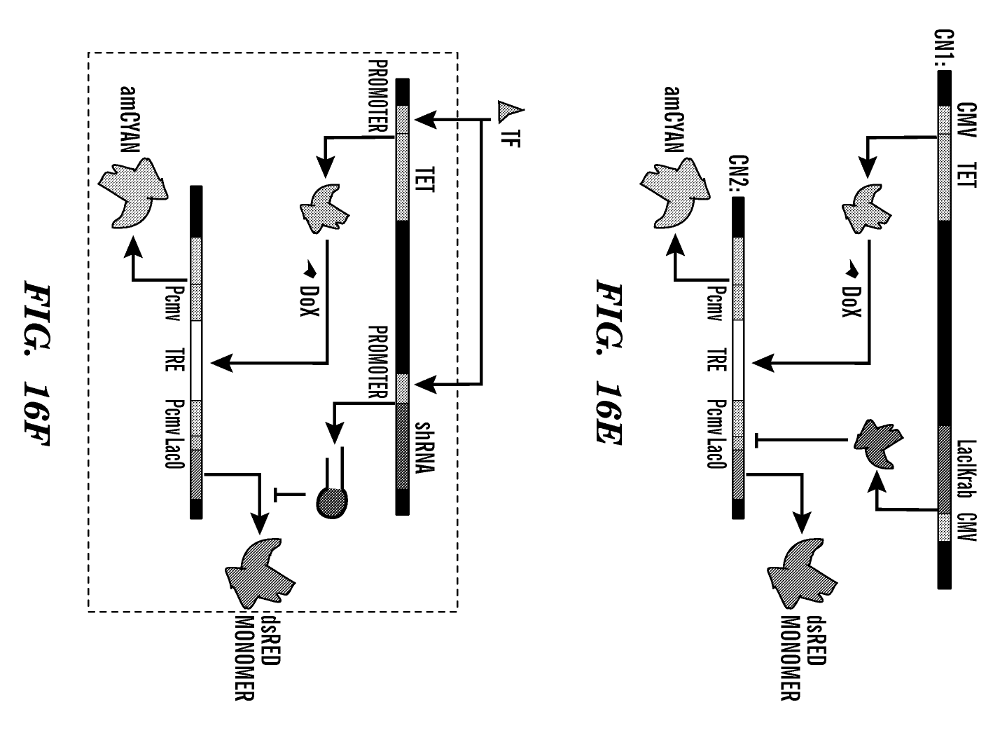

Multi-siRNA systems may fail due to crosstalk between individual molecules. This crosstalk was measured by constructing a set of zsYellow derivatives with single target sites cloned into the gene's 3′-UTR and applying all siRNA molecules at the saturation concentration, one at a time, to each construct. These results indicate that for this set of siRNAs crosstalk is negligible (FIG. 10), except for a possible minor (20%) reduction in the reporter level when SI4 siRNA is applied to the FF4 target; this is further reduced to ˜10% when the FF4 target is a part of a clause molecule (FIG. 11). Given these results, we proceeded to construct large-scale circuits. First, a number of clause molecules for DNF evaluators were built and tested, which clause molecules were made by fusing the siRNA targets as indicated in FIG. 5 into the 3′-UTR of the ZsYellow reporter gene. To verify circuit operation, siRNA molecules were applied to the cells at a saturation level separately to each of these constructs and the results in FIG. 11 show that downregulation is achieved separately by any of the cognate siRNAs but not by the others. Initially, one of the constructs (zsYellow-T1-SI4-FF4) showed incomplete repression by two out of three siRNAs. RNA folding analysis of the clause sequence and the alternative arrangements of the targets were performed, and it was found that an alternate arrangement, selected for its low folding energy, operates significantly better than the original (FIGS. 12A-12C). A conclusion was made that minimizing secondary structures in the multiple-target constructs is important for their robust function and is consistent with other reports on RNAi sensitivity to secondary structure. It was noted that the levels of repression observed in the crosstalk measurements is >99% in most cases, which is higher than usually obtained with RNAi. To verify the quantitative FACS data, we performed quantitative analysis of the corresponding microscope images and obtained similar results.

In the next step full Boolean expression evaluation experiments were performed. The interpretation of the siRNAs and their targets as variables in expressions are shown in Table 1.

TABLE 1
Association between the literals and the siRNAs.

| siRNA | | T1 | T2 | SI4 | FF3 | FF4 |
|---|---|---|---|---|---|---|
| (parent gene) | | (rLuc) | (rLuc) | (eGFP) | (ffLuc) | (ffLuc) |
| Expression | D1 | A | B | C | D | E |
| literals | D2 | A | B | C | NOT (A) | E |
| | C1 | — | — | — | D | E |
| | C2 | — | — | — | D | E |
| | E1 | — | — | — | D | — |

Two expressions were evaluated in DNF form,

D1: (a AND b AND c) OR (d AND e) and

D2: (a AND c AND e) OR(NOT(a) AND b).

The same siRNA (FF3) is interpreted differently in D1 and D2, once as an independent variable e and once as a negative literal NOT(a). As a result, siRNAs T1 and FF3 never appear together in the set of inputs for D2. Next, all possible truth value assignments were evaluated for the variables in each expression: 32 for the D1 (FIG. 13A) and 16 for D2 (FIG. 13A). The distribution of reporter expression levels in D1 and D2 expressions is shown in FIG. 13B. It demonstrates a nearly digital separation between the groups of FALSE and TRUE outputs as expected from a Boolean evaluator, with an average of 16-fold difference between reporter levels in FALSE and TRUE groups. The last evaluation of the D1 expression, with all variables being TRUE and no siRNAs present, results in more than twice the reporter protein level as compared to others due to the concurrent expression of the reporter from both clause molecules. Since a TRUE result is an expression level obtained from at least one clause molecule, this high value is also interpreted as TRUE. In the expression D2, one notable outlier (a:T, b:F, c:F, e:T) was obtained that was supposed to be suppressed but instead generates 0.32 expression units relative to the lowest unsuppressed (“TRUE”) reporter level. This could not be explained solely by the imperfect downregulation of the clause molecule Target (a)-(c)-(e) by SI4, as the same siRNA works about twice as efficiently in other evaluation experiments and three times as efficiently in the experiment shown in FIG. 10, where SI4 is applied at the same concentration. On the other hand, increasing the amount of the SI4 siRNA from 2.5 pmol to 10 pmol per transfection resulted in a four-fold improvement in the repression. Similar improvement is obtained with the (a:F, b:F, c:T, e:T) evaluation that generates 0.22 units under standard conditions but may be reduced ˜four-fold by an increase in the T1 siRNA level.

We next fused siRNA targets to the 3′-UTR of the LacI repressor driven by the CMV promoter (FIG. 5A) to evaluate a single-clause CNF expression C1: (b OR d) and a two-clause, single-literal expression C2: (b) AND (d). In the latter expression, each single-literal clause molecule was modified by the triple tandem repeat of the target instead of a single occurrence to improve the repression efficiency (Sullivan et al. (2005) J. Virol. 79, 7371-7379). The dsRed-monomer reporter of the truth values in CNF expression was under the control of CAGOP promoter (Caron et al. (2005) Cell. Mol. Life Sci. 62 1605-1612) (FIG. 5B). The CNF evaluator (FIG. 5C) performs an AND operation between clauses and OR operation within a clause; however at this point, the CNF evaluator is quantitatively less robust than its DNF counterpart. This reflected a common problem in natural and synthetic networks of noise accumulation with increasing number of layers. In this particular case, robust behavior will be achieved when the repression by the transcription factor is tight on one hand, and its downregulation by the siRNA is complete, on the other. The first point was addressed by increasing the strength of the operator (CAGOP) and the second by fusing tandem repeats of the siRNA targets into the 3′-UTR of the repressor. This resulted in significantly better performance than that of a weaker operator (CMV-LacO) and single target copies.

Although the DNF form itself may represent any Boolean expression, CNF and DNF representations of the same logic function differ in size, particularly, in the total number of literals and their appearances. In the context of biological systems, each appearance of a literal means an additional siRNA target site and hence increased chances of a failure. Therefore, it was advantageous to be able to choose the simpler of the two alternatives.

An initial study of the modularity of the Boolean evaluator itself was undertaken, in particular the ability to combine different cue encoding rules with different downstream evaluator networks. For example, combining the encoding used to evaluate expressions in DNF (FIG. 5C), with the circuit designed to evaluate expressions in CNF results in a circuit that evaluates NOT(E), where E is the original DNF expression. Therefore, combining circuits designed to evaluate E and NOT(E) in the same environment renders two anti-correlated outputs that may compensate' for the imperfections of the individual evaluators, given that the outputs of the processes neutralize each other. FIG. 5D demonstrates this feature for the trivial single-literal expression E1: (d).

### Example 3

To demonstrate that a “noise-reduction motif” can control the distribution of an output of a molecular circuit, a circuit was constructed using the transcriptional regulator LacI (a DNA-binding protein that operates by binding to an operator region (i.e., Lac operon) and blocks RNA polymerase from binding. LacI was used to inhibit the expression of the output—a gene coding for the dsRed fluorescent protein (FIG. 15). The circuit also included the rtTA protein (green color), which binds to, and activates expression from TRE promoters in the presence of Doxycycline. A TRE element was placed between two minimal CMV promoters and controlled the expression of two separate genes of interest—the LacI repressor and the reporter gene dsRed monomer. As shown in FIG. 15A, the LacO binding site was positioned between the P-CMV region (a cytomegalovirus promoter) and dsRed gene. (Although the DsRed reporter could have been replaced with an arbitrary gene of interest). The molecular circuit was introduced into cells and the various components were expressed in the cell as dictated by the circuit construction. Following the expression, cells were subjected to flow cytometry analysis. The flow cytometry analysis demonstrated that cells in which the motif was active (“Motif”) exhibited a much more tightly regulated expression of the output protein dsRed, whereas cells in which the motif was turned off exhibited a more unregulated (or broader) distribution of expression of the same output protein. These results demonstrate that a “noise-reduction motif” can effectively control the distribution of an output (such as an output protein) of a molecular circuit in a cell.

### Example 4

Also a filter can effectively regulate the expression of an output of a molecular circuit. Two variants of a filter were designed for a molecular circuit, which variants are depicted in FIGS. 16C and 16E. The circuit in FIG. 16C contained the first gene cassette CN1 producing the transcriptional regulator rtTA that binds to an TRE element and activates the production of the output protein (DsRed) operably linked to the TRE, and a shRNA that binds to and results in the degradation of the mRNA encoding the same output protein. The circuit also included a second gene cassette CN2 having a TRE element between two minimal CMV promoters that controls the expression of two separate genes, in this particular case amCyan (reference gene) and the reporter gene dsRed monomer fused to an shNA target. The circuit was introduced into cells and the various circuit components were expressed as dictated by the circuit construction. When the gene copy number of the cassette CN1 increased, the band-pass filter mechanism compensated by reducing the expression of the DsRed output with respect to this copy number (see FIG. 16D). In the circuit in FIG. 16E, the gene cassette CN1 produces the transcriptional regulator rtTA and a LaclKrab transcriptional repressor that binds a LacO regulatory sequence; the second cassette was the same as above.

### Example 5

An experiment was performed to demonstrate the functionality of the exemplary circuit implementation depicted in FIG. 2A. An inactive form of the siRNA, which was prepared by annealing the antisense strand of an siRNA pair with a “protecting nucleic acid strand,” was incubated with the sense strand of the siRNA pair or, alternatively, an mRNA complementary to the “protecting nucleic acid strand” or a negative control unrelated mRNA. The antisense siRNA strand was labeled with a fluorescent dye. The incorporation of the antisense siRNA strand in inactive and active siRNA duplexes was visualized using polyacrylamide gel electrophoresis. The presence of the mRNA that contains a region complementary to the “protecting nucleic acid strand” resulted the conversion of the inactive siRNA to the active siRNA pair in a concentration-dependent manner. This exchange was performed in buffer (salt solution kept at pH˜7.4) at room temperature. In contrast, the presence of negative control, unrelated mRNA did not result in the formation of active siRNA duplex.

A Drosophila embryo lysate system was used to determine whether the resulting “active” siRNA duplex was capable of facilitating RNAi in vitro. The cleavage of an mRNA complementary to the active siRNA was assayed in Drosophila embryo lysates that recapitulate the RNAi pathway. The target mRNA complementary to the active siRNA process was radioactively labeled. Alternatively, a negative control mRNA, which was not complementary to the active siRNA, was also added radiolabeled. Active siRNA (above) and the mRNA was added to the aliquots of the embryo lystate and incubated under conditions that allowed for RNAi to occur. The mRNA integrity was assayed after 2 hours of incubation at room temperature in the lysate and visualized using polyacrylamide gel electrophoresis. The results of the experiment demonstrated that the active siRNA was competent to facilitate almost complete cleavage of the target mRNA.

These results suggested demonstrate the functionality of the exemplary circuit implementation depicted in FIG. 2A.

### REFERENCES

1. Benenson, Y., Gil, B., Ben-Dor, U., Adar, R., Shapiro, E. An autonomous molecular computer for logical control of gene expression. Nature 429, 423-429 (2004).
2. Isaacs, F. J. et al. Engineered riboregulators enable post-transcriptional control of gene expression. Nat. Biotechnol. 22, 841-847 (2004).
3. Penchovsky. R., Breaker, R. R. Computational design and experimental validation of oligonucleotide-sensing allosteric ribozymes. Nat. Biotechnol. 23, 1424-1433 (2005).
4. Bayer, T. S., Smolke, C. D. Programmable ligand-controlled riboregulators of eukaryotic gene expression. Nat. Biotechnol. 23, 337-343 (2005).
5. Adleman, L. M. Molecular computation of solutions to combinatorial problems. Science 266, 1021-1024 (1994).
6. Winfree, E., Liu, F. R., Wenzler, L. A., Seeman, N. C. Design and self-assembly of two-dimensional DNA crystals. Nature 394, 539-544 (1998).
7. Benenson, Y. et al. Programmable and autonomous computing machine made of biomolecules. Nature 414, 430-434 (2001).
8. Stojanovic, M. N., Stefanovic, D. A deoxyribozyme-based molecular automaton. Nat. Biotechnol. 21, 1069-1074 (2003).
9. Baron, R., Lioubashevski, O., Katz, E., Niazov, T., Willner, I. Elementary arithmetic operations by enzymes: A model for metabolic pathway based computing. Angew. Chem. Int. Ed. 45, 1572-1576 (2006).
10. Weiss, R., Homsy, G. E., Knight, T. F. Toward in vivo digital circuits. DIMACS workshop on evolution in computation (1999).
11. Kramer, B. P., Fischer, C., Fussenegger, M. BioLogic gates enable logical transcription control in mammalian cells. Biotech and Bioeng. 87, 478-484 (2004).
12. Seelig, G., Soloveichik, D., Zhang, D. Y., Winfree, E. Enzyme-free nucleic acid logic circuits. Science 314, 1585-1588 (2006).
13. Eckstein, F. Small non-coding RNAs as magic bullets. Trends Biochem. Sci. 30, 445-452 (2005).
14. Kobayashi, H. et al. Programmable cells: Interfacing natural and engineered gene networks. Proc. Natl. Acad. Sci. USA 101, 4814-4819 (2004).
15. Elowitz, M., Leibler, S. A synthetic oscillatory network of transcriptional regulators. Nature 403, 335-338 (2000).
16. Basu, S., Gerchman, Y., Collins, C. H., Arnold, F. H., Weiss, R. A synthetic multicellular system for programmed pattern formation. Nature 434, 1130-1134 (2005).
17. Balzani, V., Credi, A., Venturi, M. Molecular logic circuits. Chem Phys Chem 4, 49-59 (2003).
18. Soloveichik, D., Winfree, E. The computational power of Benenson automata. Theor. Comp. Sci. 244, 279-297 (2005).
19. Buchler, N. E., Gerland, U., Hwa, T. On schemes of combinatorial transcription logic. Proc. Natl. Acad. Sci. USA 100, 5136-41 (2003).
20. An, C. I., Trinh, V. B., Yokobayashi, Y. Artificial control of gene expression in mammalian cells by modulating RNA interference through aptamer-small molecule interaction. RNA 12, 710-716 (2006).
21. Isaacs, F. J., Dwyer, D. J., Collins, J. J. RNA synthetic biology. Nat. Biotechnol. 24, 545-554 (2006).
22. Giraldez, A. Z. et al. Zebrafish MiR-430 promotes deadenylation and clearance of maternal mRNAs. Science 312, 75-79 (2006).
23. Caron, L. et al. The Lac repressor provides a reversible gene expression system in undifferentiated and differentiated embryonic stem cell. Cell. Mol. Life Sci. 62 1605-1612 (2005).
24. Elbashir, S. M. et al. Duplexes of 21-nucleotide RNAs mediate RNA interference in cultured mammalian cells. Nature 411, 494-498 (2001).
25. Kobayashi, N. et al. Vector-based in vivo RNA interference: Dose- and time-dependent suppression of transgene expression. J. Pharm. Exp. Therap. 308, 688-693 (2004).
26. Reynolds, A. et al. Rational siRNA design for RNA interference. Nat. Biotechnol. 22, 326-330 (2004).
27. Sullivan, C. S., Ganem, D. A virus-encoded inhibitor that blocks RNA interference in mammalian cells. J. Virol. 79, 7371-7379 (2005).
28. Schwarz, D. S. et al. Asymmetry in the Assembly of the RNAi Enzyme Complex. Cell 115, 199-208 (2003).
29. Kolpashnikov, D. M., Stojanovic, M. N. Boolean control of aptamer binding sites. J. Amer. Chem. Soc. 127, 11384-11351 (2005).
30. Beyer, S., Simmel, F. C. A modular DNA signal translator for the controlled release of a protein by an aptamer. Nucleic Acids Res. 34, 1581-1587 (2006).
31. Mao, C., Sun, W., Shen, Z., Seeman, N. C. A nanomechanical device based on the B-Z transition of DNA. Nature 397, 144-146 (1999).

1. A modified eukaryotic cell comprising an exogenous molecular circuit, wherein the molecular circuit is coupled an input cue and generates an output, the molecular circuit comprising:
(1) a plurality of mediators, the activity of each mediator being a function of an input cue;
(2) two or more molecular switches coupled to the mediators, the switches being configured to perform Boolean operations wherein the input cues are operands; and
(3) an output that is regulated by the switches.

2. The modified cell of claim 1, wherein the mediators regulate mRNA expression of the output.

3. The modified cell of claim 1, wherein at least one mediator is an siRNA, an shRNA, or an miRNA.

4. The modified cell of claim 1, wherein the input cue is a protein, a small molecule, or an mRNA.

5. The modified cell of claim 1, wherein the input cue is the output of another molecular circuit in the cell.

6. The modified cell of claim 1, wherein the circuit is coupled to more than one input cue.

7. The modified cell of claim 1, wherein the output is regulated by a regulatory protein, the regulatory protein being regulated by at least one mediator.

8. The modified cell of claim 1, wherein the output is a protein or an mRNA.

9. The modified cell of claim 12, wherein the output alters a cellular activity.

10. The modified cell of claim 1, wherein the circuit generates more than one output.

11. The modified cell of claim 1, wherein the cell is a yeast cell, a plant cell, or an animal cell.

12. The modified cell of claim 1, wherein the molecular circuit comprises more than one output.

13. The modified cell of claim 1, wherein the molecular circuit comprises more than one mediator.

14. The modified cell of claim 1, wherein the molecular circuit comprises a Boolean AND switch, a Boolean OR switch, or both a Boolean AND switch and a Boolean OR switch.

15. The modified cell of claim 1, wherein the molecular circuit implements a Boolean expression in a conjunctive normal form (CNF) or in a disjunctive normal form (DNF).

16. The modified cell of claim 1, wherein at least one input cue is an endogenous input cue or an exogenous input cue.

17. The modified cell of claim 1, wherein the cell comprises:
(1) a plurality of siRNA mediators, the activity of each siRNA mediator being responsive to an input cue; and
(2) an mRNA that encodes an output, the mRNA comprising response elements for each of the siRNA mediators.

18. The modified cell of claim 1, wherein the cell comprises:
(1) at least two miRNA mediators; and
(2) an mRNA that encodes an output, the mRNA comprising response elements for each of the miRNA mediators.

19. A modified eukaryotic cell comprising an exogenous molecular circuit, wherein the molecular circuit is coupled to at least two input cues and generates an output and wherein the input cues are evaluated in parallel, the molecular circuit comprising:
(1) a plurality of mediators, the activity of each mediator being a function of an input cue; and
(2) an output that is regulated by the mediators, wherein the output is the result of a Boolean operation for which the input cues are operands.

20. A modified eukaryotic cell comprising an exogenous molecular circuit, wherein the molecular circuit is coupled an input cue and generates an output and wherein the input cues are evaluated in parallel, the molecular circuit comprising:
(1) at least four mediators, the activity of each mediator being a function of an input cue; and
(2) an output that is regulated by the mediators, wherein the output is the result of a Boolean operation for which the input cues are operands.


SEQUENCE LISTING (58 sequences)

SEQ ID NO: 1 - RNA, 22 nt, Artificial Sequence
  Description of Artificial Sequence Synthetic oligonucleotide
cguacgcggaauacuucgaauu

SEQ ID NO: 2 - RNA, 22 nt, Artificial Sequence
  Description of Artificial Sequence Synthetic oligonucleotide
uucgaaguauuccgcguacgug

SEQ ID NO: 3 - RNA, 22 nt, Artificial Sequence
  Description of Artificial Sequence Synthetic oligonucleotide
cguugcuaguaccaacccuauu

SEQ ID NO: 4 - RNA, 22 nt, Artificial Sequence
  Description of Artificial Sequence Synthetic oligonucleotide
uaggguugguacuagcaacgcu

SEQ ID NO: 5 - RNA, 22 nt, Artificial Sequence
  Description of Artificial Sequence Synthetic oligonucleotide
ggcaagcugacccugaaguuug

SEQ ID NO: 6 - RNA, 22 nt, Artificial Sequence
  Description of Artificial Sequence Synthetic oligonucleotide
aacuucagggucagcuugccgu

SEQ ID NO: 7 - RNA, 22 nt, Artificial Sequence
  Description of Artificial Sequence Synthetic oligonucleotide
cgauaugggcugaauacaaauu

SEQ ID NO: 8 - RNA, 22 nt, Artificial Sequence
  Description of Artificial Sequence Synthetic oligonucleotide
uuuguauucagcccauaucguu

SEQ ID NO: 9 - RNA, 22 nt, Artificial Sequence
  Description of Artificial Sequence Synthetic oligonucleotide
gcuugaagucuuuaauuaaauu

SEQ ID NO: 10 - RNA, 22 nt, Artificial Sequence
  Description of Artificial Sequence Synthetic oligonucleotide
uuuaauuaaagacuucaagcgg

SEQ ID NO: 11 - DNA, 43 bp, Artificial Sequence
  Description of Artificial Sequence Synthetic oligonucleotide
tcgagcttaacaagcttcgacacgtacgcggaatacttcgaag

SEQ ID NO: 12 - DNA, 43 bp, Artificial Sequence
  Description of Artificial Sequence Synthetic oligonucleotide
gatccttcgaagtattccgcgtacgtgtcgaagcttgttaagc

SEQ ID NO: 13 - DNA, 43 bp, Artificial Sequence
  Description of Artificial Sequence Synthetic oligonucleotide
tcgagcttaacaagcttcgaagcgttgctagtaccaaccctag

SEQ ID NO: 14 - DNA, 43 bp, Artificial Sequence
  Description of Artificial Sequence Synthetic oligonucleotide
gatcctagggttggtactagcaacgcttcgaagcttgttaagc

SEQ ID NO: 15 - DNA, 43 bp, Artificial Sequence
  Description of Artificial Sequence Synthetic oligonucleotide
tcgagcttaacaagcttcgaacggcaagctgaccctgaagttg

SEQ ID NO: 16 - DNA, 43 bp, Artificial Sequence
  Description of Artificial Sequence Synthetic oligonucleotide
gatccaacttcagggtcagcttgccgttcgaagcttgttaagc

SEQ ID NO: 17 - DNA, 43 bp, Artificial Sequence
  Description of Artificial Sequence Synthetic oligonucleotide
tcgagcttaacaagcttcgaaacgatatgggctgaatacaaag

SEQ ID NO: 18 - DNA, 43 bp, Artificial Sequence
  Description of Artificial Sequence Synthetic oligonucleotide
gatcctttgtattcagcccatatcgtttcgaagcttgttaagc

SEQ ID NO: 19 - DNA, 43 bp, Artificial Sequence
  Description of Artificial Sequence Synthetic oligonucleotide
tcgagcttaacaagcttcgaccgcttgaagtctttaattaaag

SEQ ID NO: 20 - DNA, 43 bp, Artificial Sequence
  Description of Artificial Sequence Synthetic oligonucleotide
gatcctttaattaaagacttcaagcggtcgaagcttgttaagc

SEQ ID NO: 21 - DNA, 87 bp, Artificial Sequence
  Description of Artificial Sequence Synthetic oligonucleotide
tcgagcttaacaagcttcgaaacgatatgggctgaatacaaaaacgatatgggctgaatacaaaaacgatatgggctgaatacaaag

SEQ ID NO: 22 - DNA, 87 bp, Artificial Sequence
  Description of Artificial Sequence Synthetic oligonucleotide
gatcctttgtattcagcccatatcgtttttgtattcagcccatatcgtttttgtattcagcccatatcgtttcgaagcttgttaagc

SEQ ID NO: 23 - DNA, 87 bp, Artificial Sequence
  Description of Artificial Sequence Synthetic oligonucleotide
tcgagcttaacaagcttcgaccgcttgaagtctttaattaaaccgcttgaagtctttaattaaaccgcttgaagtctttaattaaag

SEQ ID NO: 24 - DNA, 87 bp, Artificial Sequence
  Description of Artificial Sequence Synthetic oligonucleotide
gatcctttaattaaagacttcaagcggtttaattaaagacttcaagcggtttaattaaagacttcaagcggtcgaagcttgttaagc

SEQ ID NO: 25 - DNA, 87 bp, Artificial Sequence
  Description of Artificial Sequence Synthetic oligonucleotide
tcgagcttaacaagcttcgacacgtacgcggaatacttcgaaagcgttgctagtaccaaccctaacggcaagctgaccctgaagttg

SEQ ID NO: 26 - DNA, 87 bp, Artificial Sequence
  Description of Artificial Sequence Synthetic oligonucleotide
gatccaacttcagggtcagcttgccgttagggttggtactagcaacgctttcgaagtattccgcgtacgtgtcgaagcttgttaagc

SEQ ID NO: 27 - DNA, 65 bp, Artificial Sequence
  Description of Artificial Sequence Synthetic oligonucleotide
tcgagcttaacaagcttcgaaacgatatgggctgaatacaaaccgcttgaagtctttaattaaag

SEQ ID NO: 28 - DNA, 65 bp, Artificial Sequence
  Description of Artificial Sequence Synthetic oligonucleotide
gatcctttaattaaagacttcaagcggtttgtattcagcccatatcgtttcgaagcttgttaagc

SEQ ID NO: 29 - DNA, 87 bp, Artificial Sequence
  Description of Artificial Sequence Synthetic oligonucleotide
tcgagcttaacaagcttcgaccgcttgaagtctttaattaaacacgtacgcggaatacttcgaaacggcaagctgaccctgaagttg

SEQ ID NO: 30 - DNA, 87 bp, Artificial Sequence
  Description of Artificial Sequence Synthetic oligonucleotide
gatccaacttcagggtcagcttgccgtttcgaagtattccgcgtacgtgtttaattaaagacttcaagcggtcgaagcttgttaagc

SEQ ID NO: 31 - DNA, 65 bp, Artificial Sequence
  Description of Artificial Sequence Synthetic oligonucleotide
tcgagcttaacaagcttcgaaacgatatgggctgaatacaaaagcgttgctagtaccaaccctag

SEQ ID NO: 32 - DNA, 65 bp, Artificial Sequence
  Description of Artificial Sequence Synthetic oligonucleotide
gatcctagggttggtactagcaacgcttttgtattcagcccatatcgtttcgaagcttgttaagc

SEQ ID NO: 33 - DNA, 28 bp, Artificial Sequence
  Description of Artificial Sequence Synthetic primer
ccagctagcgaggtaccctcccaccatg

SEQ ID NO: 34 - DNA, 33 bp, Artificial Sequence
  Description of Artificial Sequence Synthetic primer
ccaagatcttcaaaccttcctcttcttcttagg

SEQ ID NO: 35 - DNA, 21 bp, Artificial Sequence
  Description of Artificial Sequence Synthetic primer
cgtcaatgggagtttgttttg

SEQ ID NO: 36 - DNA, 20 bp, Artificial Sequence
  Description of Artificial Sequence Synthetic primer
gcgccgagacagaacttaat

SEQ ID NO: 37 - DNA, 40 bp, Artificial Sequence
  Description of Artificial Sequence Synthetic primer
actaggttaattaatagttattaatagtaatcaattacgg

SEQ ID NO: 38 - DNA, 81 bp, Artificial Sequence
  Description of Artificial Sequence Synthetic primer
gatgaagaattcaggccgaggcggccgtcgacgttaacgctagcggccgtaatggcctacctgtgggagtaacgcggtcag

SEQ ID NO: 39 - DNA, 45 bp, Artificial Sequence
  Description of Artificial Sequence Synthetic primer
actaagcacctgcactccaggaacgcacgggtgttgggtcgtttg

SEQ ID NO: 40 - DNA, 44 bp, Artificial Sequence
  Description of Artificial Sequence Synthetic primer
ctagatcacctgcatcactgcccgctttccagtcgggaaacctg

SEQ ID NO: 41 - DNA, 45 bp, Artificial Sequence
  Description of Artificial Sequence Synthetic primer
ctaatccacctgcactcgcagccaaaaaagaagagaaaggtcgac

SEQ ID NO: 42 - DNA, 48 bp, Artificial Sequence
  Description of Artificial Sequence Synthetic primer
atcatccacctgcatcacctgttaaactgatgatttgatttcaaatgc

SEQ ID NO: 43 - DNA, 34 bp, Artificial Sequence
  Description of Artificial Sequence Synthetic primer
actactgctagctcccaccatgaaaccagtaacg

SEQ ID NO: 44 - DNA, 38 bp, Artificial Sequence
  Description of Artificial Sequence Synthetic primer
catcatagatctttaaactgatgatttgatttcaaatg

SEQ ID NO: 45 - DNA, 73 bp, Artificial Sequence
  Description of Artificial Sequence Synthetic oligonucleotide
tcgagcttaacaagcttcgaaacgatatgggctgaatacaaaaacgatatgggctgaatacaaaaacgatatg

SEQ ID NO: 46 - DNA, 74 bp, Artificial Sequence
  Description of Artificial Sequence Synthetic oligonucleotide
cagcccatatcgtttttgtattcagcccatatcgtttttgtattcagcccatatcgtttcgaagcttgttaagc

SEQ ID NO: 47 - DNA, 80 bp, Artificial Sequence
  Description of Artificial Sequence Synthetic oligonucleotide
ggctgaatacaaaccgcttgaagtctttaattaaaccgcttgaagtctttaattaaaccgcttgaagtctttaattaaag

SEQ ID NO: 48 - DNA, 79 bp, Artificial Sequence
  Description of Artificial Sequence Synthetic oligonucleotide
gatcctttaattaaagacttcaagcggtttaattaaagacttcaagcggtttaattaaagacttcaagcggtttgtatt

SEQ ID NO: 49 - DNA, 81 bp, Artificial Sequence
  Description of Artificial Sequence Synthetic oligonucleotide
cttaacaagcttcgacacgtacgcggaatacttcgaaagcgttgctagtaccaaccctaacggcaagctgaccctgaagtt

SEQ ID NO: 50 - DNA, 59 bp, Artificial Sequence
  Description of Artificial Sequence Synthetic oligonucleotide
cttaacaagcttcgaaacgatatgggctgaatacaaaagcgttgctagtaccaacccta

SEQ ID NO: 51 - DNA, 81 bp, Artificial Sequence
  Description of Artificial Sequence Synthetic oligonucleotide
cttaacaagcttcgaccgcttgaagtctttaattaaacacgtacgcggaatacttcgaaacggcaagctgaccctgaagtt

SEQ ID NO: 52 - DNA, 59 bp, Artificial Sequence
  Description of Artificial Sequence Synthetic oligonucleotide
cttaacaagcttcgaaacgatatgggctgaatacaaaccgcttgaagtctttaattaaa

SEQ ID NO: 53 - RNA, 87 nt, Artificial Sequence
  Description of Artificial Sequence Synthetic oligonucleotide
ucgagcuuaacaagcuucgacacguacgcggaauacuucgaaacggcaagcugacccugaaguuccgcuugaagucuuuaauuaaag

SEQ ID NO: 54 - RNA, 87 nt, Artificial Sequence
  Description of Artificial Sequence Synthetic oligonucleotide
ucgagcuuaacaagcuucgaacggcaagcugacccugaaguuccgcuugaagucuuuaauuaaacacguacgcggaauacuucgaag

SEQ ID NO: 55 - RNA, 87 nt, Artificial Sequence
  Description of Artificial Sequence Synthetic oligonucleotide
ucgagcuuaacaagcuucgaccgcuugaagucuuuaauuaaaacggcaagcugacccugaaguucacguacgcggaauacuucgaag

SEQ ID NO: 56 - RNA, 87 nt, Artificial Sequence
  Description of Artificial Sequence Synthetic oligonucleotide
ucgagcuuaacaagcuucgaacggcaagcugacccugaaguucacguacgcggaauacuucgaaccgcuugaagucuuuaauuaaag

SEQ ID NO: 57 - RNA, 86 nt, Artificial Sequence
  Description of Artificial Sequence Synthetic oligonucleotide
ucgagcuuaacaagcuucgacacguacgcggaauacuucgaaccgcuugaagucuuuaauuaaaacggcaagcugacccugaaguu

SEQ ID NO: 58 - RNA, 87 nt, Artificial Sequence
  Description of Artificial Sequence Synthetic oligonucleotide
ucgagcuuaacaagcuucgaccgcuugaagucuuuaauuaaacacguacgcggaauacuucgaaacggcaagcugacccugaaguug